(19) United States
(12) Patent Application Publication
(10) Pub. No.: US 2026/0226037 A1
(43) Pub. Date: Aug. 6, 2026

(54) MICROTUBULE TARGETING AGENTS

(71) Applicant: ARIEL SCIENTIFIC INNOVATIONS LTD., Ariel (IL)

(72) Inventors: Gabriela ROZIC, Ariel (IL); Igor KOMAN, Ariel (IL); Merav LEIBA, Ariel (IL); Irit SHAPIRA-NETANELOV, Ariel (IL)

(21) Appl. No.: 19/117,225

(22) PCT Filed: Oct. 1, 2023
(86) PCT No.: PCT/IL2023/051052

§ 371 Date: Mar. 31, 2025

Related U.S. Application Data
(60) Provisional application No. 63/412,399, filed on Oct. 1, 2022.

(51) Int. Cl. C07D 413/04; A61K 31/404; A61K 31/422; A61K 31/427; A61K 31/437; A61P 35/00; A61P 35/02; C07D 405/04; C07D 409/04; C07D 417/04; C07D 471/04

(52) U.S. Cl. CPC C07D 413/04; A61K 31/404; A61K 31/422; A61K 31/427; A61K 31/437; A61P 35/00; A61P 35/02; C07D 405/04; C07D 409/04; C07D 417/04; C07D 471/04

(57) ABSTRACT

The present invention is directed to compounds and compositions comprising thereof. Further, methods of use such as for the treatment and prevention of cancer in a subject in need thereof are also provided.

### CROSS-REFERENCE TO RELATED APPLICATIONS

This application claims the benefit of priority of U.S. Provisional Patent Application No. 63/412,399 filed Oct. 1, 2022, the contents of which are incorporated herein by reference in their entirety.

### FIELD OF THE INVENTION

The present invention relates generally to the field of compositions comprising one or more microtubule targeting agents and is directed to methods of using the same such as for cancer treatment.

### BACKGROUND OF THE INVENTION

Cancer is one of the most common causes of death and its incidence and mortality rates are increasing rapidly worldwide. Despite the significant improvement in survival outcomes, it remains an incurable disease.

Microtubules are cytoskeletal filaments consisting of ap-tubulin heterodimers that are involved in diverse cellular functions, such as maintenance of cell shape and architecture, intracellular transport of vesicles, organelles and other components, cell signaling, cell motility, and cell division. During mitosis, attachment of the microtubules of the bipolar spindle with each individual chromosome is critical for chromosomal alignment at the metaphase plate and synchronous segregation into daughter cells. Since cancer cells divide more rapidly than normal cells, they frequently pass through a stage of vulnerability to mitotic poisons and are relatively more sensitive to microtubule dynamics changes. The suppression of spindle-microtubule dynamics by a family of anti-cancer drugs, named microtubule-targeting agents (MTAs), results in blocking mitosis at the metaphase-anaphase transition and induction of apoptotic cell death. In addition, MTAs have been shown to have selective activity against the vasculature in tumors and can cause hemorrhage and tumor necrosis. Since abnormal vasculature is a hallmark of solid tumors, it represents by itself an important target in cancer treatment. MTAs significantly increased response to chemotherapy and survival among patients with many diverse forms of cancer. However, with the time patients acquire drug resistance or toxicity which leads to decreased MTAs doses or discontinuation of the treatment and impact tumor response, prognosis, and finally overall survival of the patients.

A major problem to overcome of many microtubule-targeting agents used in the clinic is innate and acquired drug resistance. This resistance commonly arises due to overexpression of the drug efflux pump protein, P-glycoprotein (Pgp). This membrane-associated transporter is typically expressed in many patients and cancer cell lines or could be expressed in response to treatment. P-gp prevents the intracellular accumulation of different cancer drugs by actively effluxing them out of cancer cells. The poor clinical response of some patients to taxanes and vinca alkaloids can be attributed in part to the expression of Pgp. Therefore, the development of novel MTAs with the potential to overcome the mechanisms of resistance to available MTA chemo-drugs, is becoming increasingly important for cancer therapy. Another limitation of the MTAs is their high poor pharmacokinetics mainly because of their low water solubility that limit their clinical application to intravenous routes of administration. There is therefore an ongoing need for smaller molecular weight compounds with higher water solubility that would have the potential for alternative routes of delivery, particularly oral delivery.

### SUMMARY OF THE INVENTION

In one aspect of the invention, there is provided a compound or a salt thereof, wherein the compound is represented by Formula I:

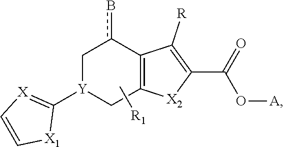

wherein: R represents a substituent comprising any one of an optionally substituted heteroaryl, an optionally substituted aryl, an optionally substituted bicyclic aromatic ring, an optionally substituted aliphatic ring, an optionally substituted unsaturated aliphatic ring, an optionally substituted bicyclic aliphatic ring, optionally substituted C₃-C₁₀ heterocyclyl, an optionally substituted linear C₁-C₁₀ alkyl, an optionally substituted branched C₁-C₁₀ alkyl, an optionally substituted C₁-C₁₀ alkyl-aryl, optionally substituted C₁-C₁₀ alkyl-cycloalkyl, optionally substituted C₃-C₁₀ cycloalkyl, an optionally substituted branched C₁-C₇ haloalkyl, an optionally substituted linear C₁-C₇ haloalkyl, an optionally substituted C₁-C₇ alkylhydroxy, an optionally substituted C₁-C₇ alkoxy, a hydroxy, a heteroatom, a halogen, an alkyne, an alkoxy, an amino, or a combination thereof; R₁ represents hydrogen, or a substituent comprising any one of an optionally substituted heteroaryl, an optionally substituted aryl, an optionally substituted bicyclic aromatic ring, an optionally substituted aliphatic ring, an optionally substituted unsaturated aliphatic ring, an optionally substituted bicyclic aliphatic ring, optionally substituted C₃-C₁₀ heterocyclyl, an optionally substituted linear C₁-C₁₀ alkyl, an optionally substituted branched C₁-C₁₀ alkyl, an optionally substituted C₁-C₁₀ alkyl-aryl, optionally substituted C₁-C₁₀ alkyl-cycloalkyl, optionally substituted C₃-C₁₀ cycloalkyl, an optionally substituted branched C₁-C₇ haloalkyl, an optionally substituted linear C₁-C₇ haloalkyl, an optionally substituted C₁-C₇ alkylhydroxy, an optionally substituted C₁-C₇ alkoxy, a hydroxy, a heteroatom, a halogen, an alkyne, an alkoxy, an amino, or a combination thereof; A represents a substituent comprising any one of an optionally substituted linear C₁-C₁₀ alkyl, an optionally substituted branched C₁-C₁₀ alkyl, an optionally substituted C₁-C₁₀ alkyl-aryl, an optionally substituted C₁-C₁₀ alkyl-cycloalkyl, an optionally substituted C₃-C₁₀ cycloalkyl, an optionally substituted branched C₁-C₇ haloalkyl, an optionally substituted linear C₁-C₇ haloalkyl, an optionally substituted C₁-C₇ alkylhydroxy, an optionally substituted unsaturated aliphatic ring, an optionally substituted C₃-C₁₀ heterocyclyl, or a combination thereof; [symbol] represents a single bond or a double bond; B is selected from O and OH; X, X₁ and X₂ are independently selected from the group consisting of: S, O, N, and NH; and Y is selected from N and CH. In one embodiment, the compound is represented by Formula I, wherein: R represents a substituent comprising any one of halogen, —C(═O)R′, —C(═O), —NO₂, —CN, —OR′, oxo, imino, —CONH₂, —CONR′₂, —CNNR′₂, —CSNR′₂, —CONH—OH, —CONH—NH₂, NHCOR′, —NHCSR′, —NHCNR′, —NC(═O)OR, —NC(═O)NR′, —NC(═S)OR′, —NC(═S)NR′, —SO₂R′, —SOR′, —SR′, —SO₂OR′, —SO₂N(R′)₂, —NHNR′₂, —NNR′, C₁-C₆ haloalkyl, optionally substituted C₁-C₆ alkyl, —NH₂, —N(R′)₂, —NR′₂—NH(C₁-C₆ alkyl), —N(C₁-C₆ alkyl)₂, C₁-C₆ alkoxy, C₁-C₆ haloalkoxy, hydroxy(C₁-C₆ alkyl), hydroxy(C₁-C₆ alkoxy), alkoxy(C₁-C₆ alkyl), alkoxy(C₁-C₆ alkoxy), C₁-C₆ alkyl-NR′₂, C₁-C₆ alkyl-SR′, —CONH(C₁-C₆ alkyl), —CON(C₁-C₆ alkyl)₂, —CO₂H, —CO₂R′, —OCOR, —OCOR′, —OC(═O)OR′, —OC(═O)NR′, —OC(═S)OR′, —OC(═S)NR′, a heteroatom, alkyne, an optionally substituted aryl, an optionally substituted bicyclic aromatic ring, an optionally substituted aliphatic ring, an optionally substituted unsaturated aliphatic ring, an optionally substituted polycyclyl, an optionally substituted bicyclic aliphatic ring, optionally substituted C₃-C₁₀ heterocyclyl, an optionally substituted linear C₁-C₁₀ alkyl, optionally substituted linear C₁-C₁₀ heteroalkyl, an optionally substituted branched C₁-C₁₀ alkyl, an optionally substituted C₁-C₁₀ alkyl-aryl, optionally substituted C₁-C₁₀ alkyl-cycloalkyl, optionally substituted C₃-C₁₀ cycloalkyl, an optionally substituted branched C₁-C₇ haloalkyl, an optionally substituted linear C₁-C₇ haloalkyl, an optionally substituted C₁-C₇ alkylhydroxy, an optionally substituted C₁-C₇ alkoxy, or a combination thereof; wherein each R′ independently represents hydrogen, or is selected from the group comprising optionally substituted C₁-C₁₀ alkyl, optionally substituted C₁-C₃₀ alkyl, optionally substituted C₁-C₃₀ alkenyl, optionally substituted C₁-C₃₀ alkynyl, optionally substituted C₃-C₁₀ cycloalkyl, optionally substituted C₃-C₁₀ heterocyclyl, optionally substituted heteroaryl, optionally substituted aryl, hydroxy, amino, —NH₂, —NR′₂—NH(C₁-C₆ alkyl), —N(C₁-C₆ alkyl)₂, C₁-C₆ alkoxy, C₁-C₆ haloalkoxy, hydroxy(C₁-C₆ alkyl), hydroxy(C₁-C₆ alkoxy), alkoxy(C₁-C₆ alkyl), alkoxy(C₁-C₆ alkoxy), C₁-C₆ alkyl-NR′₂, C₁-C₆ alkyl-SR′, or a combination thereof, R₁ represents hydrogen, or one or more substituent(s) each independently comprising any one of: C(═O)R′, —C(═O), halogen, —NO₂, —CN, —OR′, oxo, imino, —CONH₂, —CONR′₂, —CNNR′₂, —CSNR′₂, —CONH—OH, —CONH—NH₂, NHCOR′, —NHCSR′, —NHCNR′, —NC(═O)OR, —NC(═O)NR′, —NC(═S)OR′, —NC(═S)NR′, —SO₂R′, —SOR′, —SR′, —SO₂OR′, —SO₂N(R′)₂, —NHNR′₂, —NNR′, C₁-C₆ haloalkyl, optionally substituted C₁-C₆ alkyl, —NH₂, —N(R′)₂, —NR′₂—NH(C₁-C₆ alkyl), —N(C₁-C₆ alkyl)₂, C₁-C₆ alkoxy, C₁-C₆ haloalkoxy, hydroxy(C₁-C₆ alkyl), hydroxy(C₁-C₆ alkoxy), alkoxy(C₁-C₆ alkyl), alkoxy(C₁-C₆ alkoxy), C₁-C₆ alkyl-NR′₂, C₁-C₆ alkyl-SR′, —CONH(C₁-C₆ alkyl), —CON(C₁-C₆ alkyl)₂, —CO₂H, —CO₂R′, —OCOR, —OCOR′, —OC(═O)OR′, —OC(═O)NR′, —OC(═S)OR′, —OC(═S)NR′, a heteroatom, alkyne, an optionally substituted heteroaryl, an optionally substituted aryl, an optionally substituted bicyclic aromatic ring, an optionally substituted aliphatic ring, an optionally substituted unsaturated aliphatic ring, an optionally substituted bicyclic aliphatic ring, optionally substituted C₃-C₁₀ heterocyclyl, an optionally substituted polycyclyl, an optionally substituted linear C₁-C₁₀ alkyl, optionally substituted linear C₁-C₁₀ heteroalkyl, an optionally substituted branched C₁-C₁₀ alkyl, an optionally substituted C₁-C₁₀ alkyl-aryl, optionally substituted C₁-C₁₀ alkyl-cycloalkyl, optionally substituted C₃-C₁₀ cycloalkyl, an optionally substituted branched C₁-C₇ haloalkyl, an optionally substituted linear C₁-C₇ haloalkyl, an optionally substituted C₁-C₇ alkylhydroxy, an optionally substituted C₁-C₇ alkoxy, a hydroxy, a heteroatom, a halogen, an alkyne, an alkoxy, an amino, or a combination thereof; A represents a substituent comprising any one of an optionally substituted linear C₁-C₁₀ alkyl, optionally substituted linear C₁-C₁₀ heteroalkyl, an optionally substituted branched C₁-C₁₀ alkyl, an optionally substituted C₁-C₁₀ alkyl-aryl, an optionally substituted C₁-C₁₀ alkyl-cycloalkyl, an optionally substituted C₃-C₁₀ cycloalkyl, an optionally substituted branched C₁-C₇ haloalkyl, an optionally substituted linear C₁-C₇ haloalkyl, an optionally substituted C₁-C₇ alkylhydroxy, an optionally substituted unsaturated aliphatic ring, an optionally substituted C₃-C₁₀ heterocyclyl, or a combination thereof; [symbol] represents a single bond or a double bond; B is selected from O and OH; X, X₁ and X₂ are independently selected from the group consisting of: S, O, N, and NH; and Y is selected from N and CH.

In some embodiments, R represents a substituent comprising any one of —NO₂, —CN, —OH, —NH₂, carbonyl, —CONH₂, —CONR₂, —CNNR₂, —CSNR₂, —CONH—OH, —CONH—NH₂, —NHCOR, —NHCSR, —NHCNR, —NC(═O)R, —NC(═O)OR, —NC(═O)NR, —NC(═S)OR, —NC(═S)NR, —SO₂R, —SOR, —SR, —SO₂OR, —SO₂N(R)₂, —NHNR₂, —NNR, C₁-C₇ haloalkyl, optionally substituted C₁-C₁₀ alkyl, —NH(C₁-C₆ alkyl), —N(C₁-C₆ alkyl)₂, C₁-C₆ alkoxy, C₁-C₆haloalkoxy, hydroxy(C₁-C₆ alkyl), hydroxy(C₁-C₆ alkoxy), alkoxy(C₁-C₆ alkyl), alkoxy(C₁-C₆ alkoxy), amino(C₁-C₆ alkyl), —CONH(C₁-C₆ alkyl), —CON(C₁-C₆ alkyl)₂, —CO₂H, —CO₂R, —OCOR, —C(═O)R, —OC(═O)OR, —OC(═O)NR, —OC(═S)OR, —OC(═S)NR, a heteroatom, an optionally substituted cycloalkyl, an optionally substituted heterocyclyl, an optionally substituted aryl or a combination thereof.

In some embodiments, the compound is represented by Formula II:

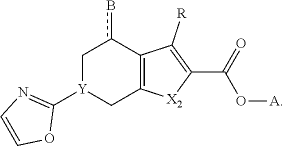

In some embodiments, X₂ is selected from 0 and NH.

In some embodiments, the compound is any one of:

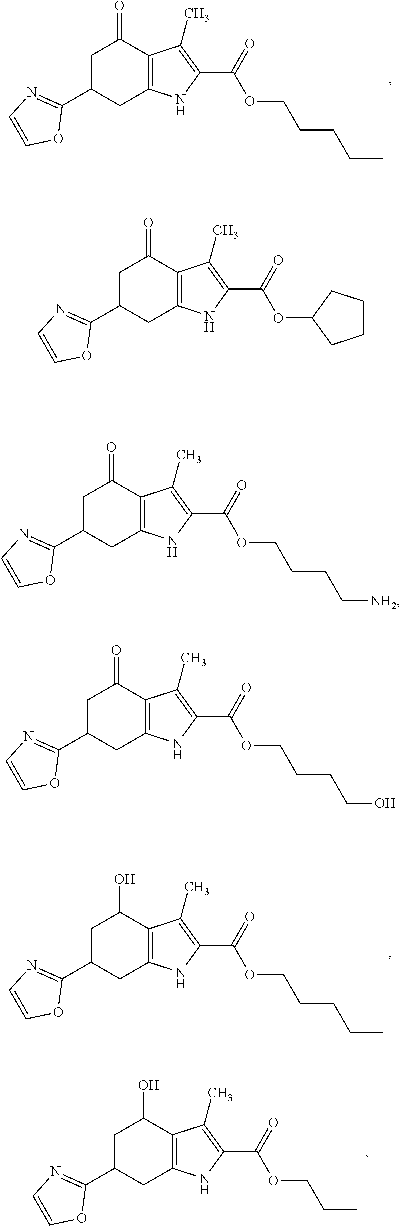 [SMILES: CCCCCOC(=O)c1[nH]c2c(c1C)C(=O)CC(c1ncco1)C2 ; Cc1c(C(=O)OC2CCCC2)[nH]c2c1C(=O)CC(c1ncco1)C2 ; Cc1c(C(=O)OCCCCN)[nH]c2c1C(=O)CC(c1ncco1)C2 ; Cc1c(C(=O)OCCCCO)[nH]c2c1C(=O)CC(c1ncco1)C2 ; CCCCCOC(=O)c1[nH]c2c(c1C)C(O)CC(c1ncco1)C2 ; CCCOC(=O)c1[nH]c2c(c1C)C(O)CC(c1ncco1)C2]

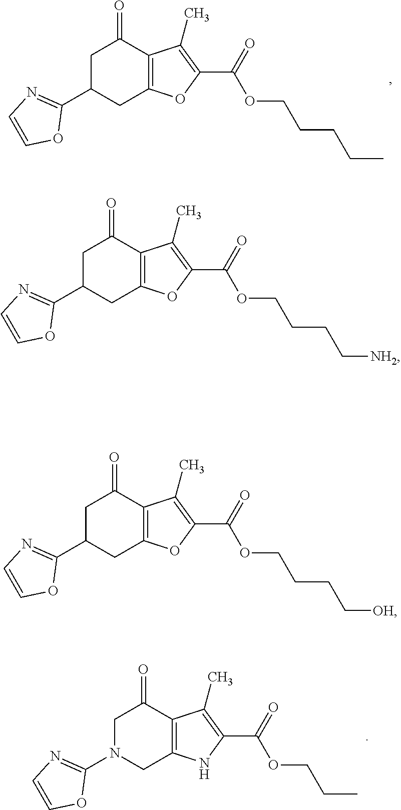 [SMILES: CCCCCOC(=O)c1oc2c(c1C)C(=O)CC(c1ncco1)C2 ; Cc1c(C(=O)OCCCCN)oc2c1C(=O)CC(c1ncco1)C2 ; Cc1c(C(=O)OCCCCO)oc2c1C(=O)CC(c1ncco1)C2 ; CCCOC(=O)c1[nH]c2c(c1C)C(=O)CN(c1ncco1)C2]

In another aspect of the invention, there is provided a pharmaceutical composition, comprising the compound of the present invention and a pharmaceutically acceptable carrier.

In some embodiments, the pharmaceutical composition is for use in the inhibition of tubulin polymerization, cell migration, endothelial cell proliferation, angiogenesis, or any combination thereof.

In some embodiments, the pharmaceutical composition is for use in the prevention or treatment of an angiogenesis-associated disease or disorder.

In some embodiments, the pharmaceutical composition is for use in the prevention or treatment of a disease or disorder associated with cancer.

In another aspect of the invention, there is provided a method for preventing or treating an angiogenesis-associated disease or disorder in a subject, comprising administering to the subject the pharmaceutical composition of the present invention, thereby preventing or treating an angiogenesis-associated disease or disorder in a subject.

In some embodiments, the disease or the disorder is cancer.

In another aspect of the invention, there is provided a method for treating or preventing development of cancer in a subject in need thereof, comprising administering to the subject the pharmaceutical composition of the present invention.

In some embodiments, the cancer is selected from the group consisting of lung cancer, breast cancer, osteosarcoma, neuroblastoma, colon adenocarcinoma, chronic myelogenous leukemia (CML), acute lymphoblastic leukemia, acute monocytic leukemia, acute myeloid leukemia (AML), acute promyelocyte leukemia (APL), sarcoma, myxoma, rhabdomyoma, fibroma, lipoma, teratoma; bronchogenic carcinoma, alveolar carcinoma, bronchial adenoma, sarcoma, lymphoma, chondromatous hamartoma, mesothelioma, esophageal cancer, stomach cancer, pancreatic cancer, small bowel cancer, large bowel cancer; kidney cancer, bladder cancer, urethra cancer, prostate cancer, testis cancer; hepatoma, cholangiocarcinoma, hepatoblastoma, angiosarcoma, hepatocellular adenoma, hemangioma, osteogenic sarcoma, fibrosarcoma, malignant fibrous histiocytoma, chondrosarcoma, Ewing's sarcoma, malignant lymphoma, multiple myeloma, malignant giant cell tumor chordoma, osteochronfroma, benign chondroma, chondroblastoma, chondromyxofibroma, osteoid osteoma, giant cell tumors, cancer of the skuli, meninges cancer, brain cancer, spinal cord cancer, uterus cancer, cervical cancer, cancer of the ovaries, vulva cancer, vagina cancer, Hodgkin's disease, non-Hodgkin's lymphoma, malignant melanoma, basal cell carcinoma, squamous cell carcinoma, Karposi's sarcoma, moles dysplastic nevi, lipoma, angioma, uterine sarcoma, cervix adenocarcinoma, ovary cancer, colorectal adenocarcinoma and dermatofibroma.

In some embodiments, the administering is by an oral administration, a topical administration, a systemic administration or any combination thereof.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1 presents micrographs of water solubility studies of the molecules with the oxazole group as compared with other chemical groups; microscope images (x40);

FIG. 2 presents micrographs of water solubility studies of the molecules with the oxazole group and different R₁-R₂ groups; microscope images (x40);

FIGS. 3A-3B are bar graphs of SMS077 cytotoxicity on cancer cells: dose response in different cancer cells types (FIG. 3A) and in different cell lines from the same type (FIG. 3B);

FIG. 4 presents IC₅₀ (half maximal inhibitory concentration) of the molecules in different types of cancer cell lines; ND: non determined;

FIGS. 5A-5H are bar graphs of growth inhibition by SMS077 (FIG. 5A), SMS101 (FIG. 5B), SMS096 (FIG. 5C), SMS088 (FIG. 5D), SMS108 (FIG. 5E), SMS291 (FIG. 5F), SMS078 (FIG. 5G) and SMS294 (FIG. 51H) of the isogenic acute lymphoblastic leukemia cancer cell lines NALM6 and N6/ADR (overexpresses Pgp glycoprotein);

FIGS. 6A-6F are bar graphs of growth inhibition by SMS182 (FIG. 6A), SMS228 (FIG. 6B) and SMS229 (FIG. 6C) of the isogenic acute lymphoblastic leukemia cancer cell lines NALM6 and N6/ADR (overexpresses Pgp glycoprotein); Vincristine (FIG. 6D), paclitaxel (FIG. 6E), and MMAE (FIG. 6F) were used as controls;

FIGS. 7A-7F are bar graphs of growth inhibition by SMS077 (FIG. 7A), SMS182 (FIG. 7B), SMS096 (FIG. 7C), SMS228 (FIG. 7D), SMS088 (FIG. 7E) and SMS229 (FIG. 7F) of the isogenic poorly differentiated uterine sarcoma cell lines MES-SA and MES-SA/DX5 (overexpresses Pgp glycoprotein);

FIGS. 8A-8G are bar graphs of growth inhibition by SMS077 (FIG. 8A), paclitaxel (FIG. 8B), vincristine (FIG. 8C), vinorelbine (FIG. 8D), colchicine (FIG. 8E), epothilone (FIG. 8F) and MMAE (FIG. 8G) of the isogenic poorly differentiated uterine sarcoma cell lines MES-SA and MES-SA/DX5 (overexpresses Pgp glycoprotein);

FIGS. 9A-9D are bar graphs of growth inhibition by SMS077 (FIG. 9A) and vincristine (FIG. 9B) of the isogenic multiple myeloma cells RPNI8226 (RPMI-S), RPMI-LR5 (melphalan resistance cells, overexpresses Pgp glycoprotein) and RPMI-DOX40 (doxorubicin resistant cells, overexpresses Pgp glycoprotein) and metastatic breast adenocarcinoma cells MCF-7 and MCF-7VP (FIGS. 9C-D) (etoposide resistant cells, overexpresses Pgp glycoprotein);

FIGS. 10A-10D are bar graphs of growth inhibition by SMS077 and vincristine of the isogenic (FIGS. 10A-B) ovary carcinoma cells A2780 and A2780ADR (melphalan and vinblastine cross-resistance cells) and (FIGS. 10C-D) lung adenocarcinoma cells H69 and NCI-H69 VCR/R (vincristine resistant cells);

FIG. 11 presents tubulin polymerization assay in a cell free system: purified tubulin was polymerized in the absence (DMSO, 0 μM) or presence of the molecules (1, 15, 30 μM); polymerization was detected as changes in fluorescence and monitored at 37° C. every 30 sec for 100 min;

FIGS. 12A-12B present tubulin polymerization assay in a cell system: western blotting of polymerized tubulin in RPMI8226-treated cells (FIG. 12A); the cells were treated with vehicle (DMSO, C) or different concentrations of SMS001, SMS077 or SMS182 for 18 h. Soluble and polymerized tubulin were collected and examined by western blotting; and Bar graphs (FIG. 12B) represent quantification of the of scanned images using ImageJ. Image densities were normalized against Gapdh and the ratio between polymerized and soluble tubulin calculated;

FIGS. 13A-13B present tubulin polymerization assay in a cell system: western blotting of polymerized tubulin in RPMI8226-treated cells (FIG. 13A); the cells were treated with vehicle (DMSO, C) or different concentrations of SMS088, SMS101, SMS108, SMS096, SMS228 or SMS229 for 18 h. Soluble and polymerized tubulin were collected and examined by western blotting; and bar graphs (FIG. 13B) represent quantification of the of scanned images using Image J. Images densities were normalized against Gapdh and the ratio between polymerized and soluble tubulin calculated;

FIG. 14 presents competition assay of the molecules with N,N′-ethylene-bis(iodoacetamide) on RPMI8226 cells and their effects on the inhibition of the bisthioalkylation of Cys-239 and Cys-354 of P-tubulin by N,N′-ethylene-bis(iodoacetamide); colchicine and podophillotoxin were used as controls;

FIGS. 15A-15B present competition assay of SMS001 and SMS077 with N,N′-ethylene-bis(iodoacetamide) on NALM6 cells and their effects on the inhibition of the bisthioalkylation of Cys-239 and Cys-354 of P-tubulin by N,N′-ethylene-bis(iodoacetamide) in NALM6 (FIG. 15A) and HELA (FIG. 15B) cells; colchicine, paclitaxel and vincristine were used as controls;

FIGS. 16A-16B present graphs of cell cycle distribution of treated cells: RPMI8226 cells were treated with vehicle (DMSO, Control) or different concentrations of SMS077 for 6 h (left) or 18 hours and the DNA content/cell cycle distribution was assessed by flow cytometry (FIG. 16A) and cells were treated for 6 hours with SMS182, SMS101,SMS108, SMS088, SMS096 and SMS228 and DNA content/cell cycle distribution was assessed by flow cytometry (FIG. 16B);

FIGS. 17A-17C present micrographs of the dose response of MES-SA and MES-SA/DX5 cells (FIG. 17A), NALM6 and NALM6/ADR cells (FIG. 17B), and RPMI-S and RPMI-DOX40 cells (FIG. 17C) treated with graded concentration of SMS077 during 72 hours and stained with FITC-annexin indicative of apoptosis and propidium iodide indicative of necrosis; SMS077 induces apoptosis and necrosis of treated cells;

FIGS. 18A-18B present inhibition of MDA-MB-231 cells migration by SMS077 and SMS182 in a wound scratch assay; the scratch was created mechanically in the confluent monolayer and then the cells were further incubated in vehicle- or SMS077 and SMS182-containing medium at the indicated concentrations for 36 h. Representative phase-contrast images of scratch wounds taken at time 0 h and 36 h (10x magnification) (FIG. 18A) and graphs of relative wound density quantification data was acquired every 3 hours and analyzed using Incucyte software (FIG. 18B);

FIGS. 19A-19B present inhibition of BXPC3 cells migration by SMS077 and SMS182 in a wound scratch assay; the scratch was created mechanically in the confluent monolayer and then the cells were further incubated in vehicle- or SMS077 and SMS182-containing medium at the indicated concentrations for 36 h. Representative phase-contrast images of scratch wounds taken at time 0 h and 36 h (10x magnification) (FIG. 19A) and relative wound density quantification data was acquired every 3 hours and analyzed using Incucyte software (FIG. 19B);

FIGS. 20A-20B present inhibition of HELA cells migration by SMS077 and SMS182 in a wound scratch assay; the scratch was created mechanically in the confluent monolayer and then the cells were further incubated in vehicle- or SMS077 and SMS182-containing medium at the indicated concentrations for 36 h. Representative phase-contrast images of scratch wounds taken at time 0 h and 36 h (10× magnification) (FIG. 20A) and relative wound density quantification data was acquired every 3 hours and analyzed using Incucyte software (FIG. 20B).

FIGS. 21A-21C present micrographs of HUVEC cells seeded on matrigel and treated with SMS077 and SMS182 at the indicated concentrations, for 4 h (FIG. 21A), HUVEC cells seeded on matrigel, and cultured to form vessel-like capillary structures for 4 h (FIG. 21B). Cells were then treated with SMS077 and SMS182 for 2 h. Cells were visualized using Calcein AM as stain and fluorescence microscopy. Graphs of cells viability of HUVEC cells treated for 4 h, 24 h and 48 h with SMS077 and SMS182 performed by XTT assay (FIG. 21C). SMS077 and SMS182 inhibit neovessel formation and pre-established vasculature in human endothelial HUVEC cells;

FIGS. 22A-22B present inhibition of HUVEC cells migration by SMS077 and SMS182 in a wound scratch assay. The scratch was created mechanically in the confluent monolayer and then the cells were further incubated in vehicle- or SMS077 and SMS182-containing medium at the indicated concentrations for 36 h. Representative phase-contrast images of scratch wounds taken at time 0 h, 3 h, 6 h, 9 h,12 h and 24 h (10× magnification) (FIG. 22A) and graphs of relative wound density quantification data acquired every 3 hours and analyzed using Incucyte software (FIG. 22B);

FIGS. 23A-23C present graphs of in vivo antitumor activity of SMS077 administered intraperitoneally (0, 0.5, 2 mg/kg) in myeloma xenograft mouse model. During treatment period, tumor volume (FIG. 23A) and mouse body weight (FIG. 23B) were measured once every 3-4 days. At the end of experiments, mice were sacrificed, and tumor dissected and weighed (FIG. 23C). Data are expressed as the mean of tumor weight (g)±SEM from four-five mice;

FIGS. 24A-24C present graphs of in vivo antitumor activity of SMS182 administered intraperitoneally (0, 0.5, 2 mg/kg) in myeloma xenograft mouse model. During treatment period, tumor volume (FIG. 24A) and mouse body weight (FIG. 24B) were measured once every 3-4 days. At the end of experiments, mice were sacrificed, and tumor dissected and weighed (FIG. 24C). Data are expressed as the mean of tumor weight (g)±SEM from four-five mice; and

FIGS. 25A-25D present graphs of in vivo antitumor activity of SMS228 administered intraperitoneally (0, 15, 30, 50 mg/kg) or subcutaneously (0, 30 sc mg/kg) in myeloma xenograft mouse model. During treatment period, tumor volume (FIG. 25A) and mouse body weight (FIG. 25B) were measured once every 3-4 days. At the end of experiments, mice were sacrificed and tumor dissected and weighed (FIGS. 25C-25D). Data are expressed as the mean of tumor weight (g)±SEM from three-five mice.

### DETAILED DESCRIPTION OF THE INVENTION

According to some embodiments, the present invention provides a compound or a salt thereof, wherein the compound is represented by Formula Ib:

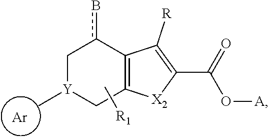

wherein:
Ar represents an optionally substituted aromatic ring (i.e. an aryl, a substituted aryl, a heteroaryl, a substituted heteroaryl, or an optionally substituted polycyclic aromatic ring), a C₃-C₅ cycloalkyl, a substituted C₃-C₅ cycloalkyl, an alkaryl, a substituted alkaryl, a bicyclic aromatic ring, a substituted bicyclic aromatic ring, a bicyclic heteroaryl, a substituted bicyclic heteroaryl, a bicyclic heterocyclyl, a substituted bicyclic heterocyclyl, a bicyclic cycloalkyl, a substituted bicyclic cycloalkyl, or a combination thereof;
R is H or represents a substituent comprising any one of halogen, —C(═O)R′, —C(═O), —NO₂, —CN, —OR′, oxo, imino, —CONH₂, —CONR′₂, —CNNR′₂, —CSNR′₂, —CONH—OH, —CONH—NH₂, NHCOR′, —NHCSR′, —NHCNR′, —NC(═O)OR, —NC(═O)NR′, —NC(═S)OR′, —NC(═S)NR′, —SO₂R′, —SOR′, —SR′, —SO₂OR′, —SO₂N(R′)₂, —NHNR′₂, —NNR′, C₁-C₆ haloalkyl, optionally substituted C₁-C₆ alkyl, —NH₂, —N(R′)₂, —NR′₂—NH(C₁-C₆ alkyl), —N(C₁-C₆ alkyl)₂, C₁-C₆ alkoxy, C₁-C₆ haloalkoxy, hydroxy(C₁-C₆ alkyl), hydroxy(C₁-C₆ alkoxy), alkoxy(C₁-C₆ alkyl), alkoxy(C₁-C₆ alkoxy), C₁-C₆ alkyl-NR′₂, C₁-C₆ alkyl-SR′, —CONH(C₁-C₆ alkyl), —CON(C₁-C₆ alkyl)₂, —CO₂H, —CO₂R′, —OCOR, —OCOR′, —OC(═O)OR′, —OC(═O)NR′, —OC(═S)OR′, —OC(═S)NR′, a heteroatom, an optionally substituted heteroaryl, an optionally substituted aryl, an optionally substituted bicyclic aromatic ring, an optionally substituted aliphatic ring, an optionally substituted unsaturated aliphatic ring, an optionally substituted bicyclic aliphatic ring, optionally substituted C₃-C₁₀ heterocyclyl, an optionally substituted polycyclyl, an optionally substituted linear C₁-C₁₀ alkyl, optionally substituted linear C₁-C₁₀ heteroalkyl, an optionally substituted branched C₁-C₁₀ alkyl, an optionally substituted C₁-C₁₀ alkyl-aryl, optionally substituted C₁-C₁₀ alkyl-cycloalkyl, optionally substituted C₃-C₁₀ cycloalkyl, an optionally substituted branched C₁-C₇ haloalkyl, an optionally substituted linear C₁-C₇ haloalkyl, an optionally substituted C₁-C₇ alkylhydroxy, an optionally substituted C₁-C₇ alkoxy, a hydroxy, a heteroatom, a halogen, an alkyne, an alkoxy, an amino, or a combination thereof; wherein each R′ independently represents hydrogen, or is selected from the group comprising optionally substituted C₁-C₁₀ alkyl, optionally substituted C₁-C₃₀ alkyl, optionally substituted C₁-C₃₀ alkenyl, optionally substituted C₁-C₃₀ alkynyl, optionally substituted C₃-C₁₀ cycloalkyl, optionally substituted C₃-C₁₀ heterocyclyl, optionally substituted heteroaryl, optionally substituted aryl, hydroxy, amino, —NH₂, —NR′₂—NH(C₁-C₆ alkyl), —N(C₁-C₆ alkyl)₂, C₁-C₆ alkoxy, C₁-C₆ haloalkoxy, hydroxy(C₁-C₆ alkyl), hydroxy(C₁-C₆ alkoxy), alkoxy(C₁-C₆ alkyl), alkoxy(C₁-C₆ alkoxy), C₁-C₆ alkyl-NR′₂, C₁-C₆ alkyl-SR′, or a combination thereof;
R₁ represents hydrogen, or one or more substituent(s) each independently comprising any one of: an optionally substituted heteroaryl, an optionally substituted aryl, an optionally substituted bicyclic aromatic ring, an optionally substituted aliphatic ring, an optionally substituted unsaturated aliphatic ring, an optionally substituted bicyclic aliphatic ring, optionally substituted C₃-C₁₀ heterocyclyl, an optionally substituted polycyclyl, an optionally substituted linear C₁-C₁₀ alkyl, optionally substituted linear C₁-C₁₀ heteroalkyl, an optionally substituted branched C₁-C₁₀ alkyl, an optionally substituted C₁-C₁₀ alkyl-aryl, optionally substituted C₁-C₁₀ alkyl-cycloalkyl, optionally substituted C₃-C₁₀ cycloalkyl, an optionally substituted branched C₁-C₇ haloalkyl, an optionally substituted linear C₁-C₇ haloalkyl, an optionally substituted C₁-C₇ alkylhydroxy, an optionally substituted C₁-C₇ alkoxy, a hydroxy, a heteroatom, a halogen, an alkyne, an alkoxy, an amino, or a combination thereof;
A represents H or a substituent comprising any one of an optionally substituted linear C₁-C₁₀ alkyl, optionally substituted linear C₁-C₁₀ heteroalkyl, an optionally substituted branched C₁-C₁₀ alkyl, an optionally substituted C₁-C₁₀ alkyl-aryl, an optionally substituted C₁-C₁₀ alkyl-cycloalkyl, an optionally substituted C₃-C₁₀ cycloalkyl, an optionally substituted branched C₁-C₇ haloalkyl, an optionally substituted linear C₁-C₇ haloalkyl, an optionally substituted C₁-C₇ alkylhydroxy, an optionally substituted unsaturated aliphatic ring, an optionally substituted C₃-C₁₀ heterocyclyl, or a combination thereof;
[symbol] represents a single bond or a double bond; B is selected from O and OH; X₂ is selected from the group consisting of: S, O, N, and NH; and Y is selected from N and CH.

In some embodiments, any one of R, A and R1 are not H. In some embodiments, Ar represents an aryl, a substituted aryl, a heteroaryl, a substituted heteroaryl, a C₃-C₈ cycloalkyl, a polycyclyl, a substituted C₃-C₈ cycloalkyl, an alkaryl, a substituted alkaryl, a bicyclic aromatic ring, a substituted bicyclic aromatic ring, a bicyclic heteroaryl, a substituted bicyclic heteroaryl, a bicyclic heterocyclyl, a substituted bicyclic heterocyclyl, a bicyclic cycloalkyl, a substituted bicyclic cycloalkyl, or a combination thereof.

The term “heteroalkyl” as used herein encompasses an alkyl having one or more (e.g. 1, 2, 3, 4 or 5) heteroatoms included in the alkyl backbone. Thus the term “heteroalkyl” encompasses at least one of S, O, N, and NR′ incorporated into alkyl backbone, such as alkyl-NH-alkyl, alkyl-O-alkyl, alkyl-S-alkyl, alkyl-NH-alkyl-O-alkyl, alkyl-O-alkyl-O-alkyl, etc.

In some embodiments, the term “polycyclic ring” or “polycyclyl” encompasses a plurality (e.g., 2, 3, 4, 5 or 6) of fused or adjacent rings (e.g., biaryl or bicyclohexyl), wherein each ring is independently selected from aryl, heteroaryl, an optionally unsaturated cycloalkyl, an optionally unsaturated heterocyclyl, or any combination thereof. In some embodiments, the term “polycyclyl” encompasses a polycyclic aromatic ring, a polycyclic aliphatic ring, or a mixed polycyclic ring.

In some embodiments, the term “mixed polycyclic ring” refers to any plurality of rings covalently bound to each other (e.g., fused rings, dicylyls, spirocyclic rings etc.) comprising at least one aromatic ring (aryl, or heteroaryl) and at least one aliphatic or nonaromatic ring (optionally a heterocyclyl and/or unsaturated cyclyl).

In some embodiments, the polycyclic ring comprises a bicyclic ring. In some embodiments, the term “bicyclic ring” encompasses a fused ring (fused aromatic and/or heteroaromatic ring), spirocyclic ring, a bridged ring, a dicyclyl (two aromatic and/or aliphatic rings joined by a single carbon-carbon bond). The terms “bicyclyl” and “bicyclic ring” are used herein interchangeably.

In some embodiments, the term “one or more” refers to any numerical value selected form of 1, 2, 3, 4, 5, or 6.

In some embodiments, the heteroatom comprises any of SR′, OR′, N(R′)₂, N, O, NH, or S, as allowed by valency.

According to some embodiments, the present invention provides a compound or a salt thereof, wherein the compound is represented by Formula I:

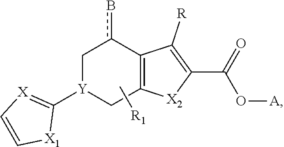

wherein: R represents a substituent comprising any one of —C(═O)R′, —C(═O), halogen, —NO₂, —CN, —OR′, oxo, imino, —CONH₂, —CONR′₂, —CNNR′₂, —CSNR′₂, —CONH—OH, —CONH—NH₂, NHCOR′, —NHCSR′, —NHCNR′, —NC(═O)OR, —NC(═O)NR′, —NC(═S)OR′, —NC(═S)NR′, —SO₂R′, —SOR′, —SR′, —SO₂OR′, —SO₂N(R′)₂, —NHNR′₂, —NNR′, C₁-C₆ haloalkyl, optionally substituted C₁-C₆ alkyl, —NH₂, —N(R′)₂, —NR′₂—NH(C₁-C₆ alkyl), —N(C₁-C₆ alkyl)₂, C₁-C₆ alkoxy, C₁-C₆ haloalkoxy, hydroxy(C₁-C₆ alkyl), hydroxy(C₁-C₆ alkoxy), alkoxy(C₁-C₆ alkyl), alkoxy(C₁-C₆ alkoxy), C₁-C₆ alkyl-NR′₂, C₁-C₆ alkyl-SR′, —CONH(C₁-C₆ alkyl), —CON(C₁-C₆ alkyl)₂, —CO₂H, —CO₂R′, —OCOR, —OCOR′, —OC(═O)OR′, —OC(═O)NR′, —OC(═S)OR′, —OC(═S)NR′, a heteroatom, alkyne, an optionally substituted aryl, an optionally substituted bicyclic aromatic ring, an optionally substituted aliphatic ring, an optionally substituted unsaturated aliphatic ring, an optionally substituted polycyclyl, an optionally substituted bicyclic aliphatic ring, optionally substituted C₃-C₁₀ heterocyclyl, an optionally substituted linear C₁-C₁₀ alkyl, optionally substituted linear C₁-C₁₀ heteroalkyl, an optionally substituted branched C₁-C₁₀ alkyl, an optionally substituted C₁-C₁₀ alkyl-aryl, optionally substituted C₁-C₁₀ alkyl-cycloalkyl, optionally substituted C₃-C₁₀ cycloalkyl, an optionally substituted branched C₁-C₇ haloalkyl, an optionally substituted linear C₁-C₇ haloalkyl, an optionally substituted C₁-C₇ alkylhydroxy, an optionally substituted C₁-C₇ alkoxy, or a combination thereof; wherein each R′ independently represents hydrogen, or is selected from the group comprising optionally substituted C₁-C₁₀ alkyl, optionally substituted C₁-C₃₀ alkyl, optionally substituted C₁-C₃₀ alkenyl, optionally substituted C₁-C₃₀ alkynyl, optionally substituted C₃-C₁₀ cycloalkyl, optionally substituted C₃-C₁₀ heterocyclyl, optionally substituted heteroaryl, optionally substituted aryl, hydroxy, amino, —NH₂, —NR′₂—NH(C₁-C₆ alkyl), —N(C₁-C₆ alkyl)₂, C₁-C₆ alkoxy, C₁-C₆ haloalkoxy, hydroxy(C₁-C₆ alkyl), hydroxy(C₁-C₆ alkoxy), alkoxy(C₁-C₆ alkyl), alkoxy(C₁-C₆ alkoxy), C₁-C₆ alkyl-NR′₂, C₁-C₆ alkyl-SR′, or a combination thereof, R₁ represents hydrogen, or one or more substituent(s) each independently comprising any one of: an optionally substituted heteroaryl, an optionally substituted aryl, an optionally substituted bicyclic aromatic ring, an optionally substituted aliphatic ring, an optionally substituted unsaturated aliphatic ring, an optionally substituted bicyclic aliphatic ring, optionally substituted C₃-C₁₀ heterocyclyl, an optionally substituted polycyclyl, an optionally substituted linear C₁-C₁₀ alkyl, optionally substituted linear C₁-C₁₀ heteroalkyl, an optionally substituted branched C₁-C₁₀ alkyl, an optionally substituted C₁-C₁₀ alkyl-aryl, optionally substituted C₁-C₁₀ alkyl-cycloalkyl, optionally substituted C₃-C₁₀ cycloalkyl, an optionally substituted branched C₁-C₇ haloalkyl, an optionally substituted linear C₁-C₇ haloalkyl, an optionally substituted C₁-C₇ alkylhydroxy, an optionally substituted C₁-C₇ alkoxy, a hydroxy, a heteroatom, a halogen, an alkyne, an alkoxy, an amino, or a combination thereof; A represents a substituent comprising any one of an optionally substituted linear C₁-C₁₀ alkyl, optionally substituted linear C₁-C₁₀ heteroalkyl, an optionally substituted branched C₁-C₁₀ alkyl, an optionally substituted C₁-C₁₀ alkyl-aryl, an optionally substituted C₁-C₁₀ alkyl-cycloalkyl, an optionally substituted C₃-C₁₀ cycloalkyl, an optionally substituted branched C₁-C₇ haloalkyl, an optionally substituted linear C₁-C₇ haloalkyl, an optionally substituted C₁-C₇ alkylhydroxy, an optionally substituted unsaturated aliphatic ring, an optionally substituted C₃-C₁₀ heterocyclyl, or a combination thereof; [symbol] represents a single bond or a double bond; B is selected from O and OH; X, X₁ and X₂ are independently selected from the group consisting of: S, O, N, and NH; and Y is selected from N and CH.

In some embodiments, the compound of the invention is represented by Formula I; wherein: R represents a substituent comprising any one of an optionally substituted heteroaryl, an optionally substituted aryl, an optionally substituted bicyclic aromatic ring, an optionally substituted aliphatic ring, an optionally substituted unsaturated aliphatic ring, an optionally substituted bicyclic aliphatic ring, optionally substituted C₃-C₁₀ heterocyclyl, an optionally substituted linear C₁-C₁₀ alkyl, an optionally substituted branched C₁-C₁₀ alkyl, an optionally substituted C₁-C₁₀ alkyl-aryl, optionally substituted C₁-C₁₀ alkyl-cycloalkyl, optionally substituted C₃-C₁₀ cycloalkyl, an optionally substituted branched C₁-C₇ haloalkyl, an optionally substituted linear C₁-C₇ haloalkyl, an optionally substituted C₁-C₇ alkylhydroxy, an optionally substituted C₁-C₇ alkoxy, a hydroxy, a heteroatom, a halogen, an alkyne, an alkoxy, an amino, or a combination thereof;
R₁ represents hydrogen, or a substituent comprising any one of an optionally substituted heteroaryl, an optionally substituted aryl, an optionally substituted bicyclic aromatic ring, an optionally substituted aliphatic ring, an optionally substituted unsaturated aliphatic ring, an optionally substituted bicyclic aliphatic ring, optionally substituted C₃-C₁₀ heterocyclyl, an optionally substituted linear C₁-C₁₀ alkyl, an optionally substituted branched C₁-C₁₀ alkyl, an optionally substituted C₁-C₁₀ alkyl-aryl, optionally substituted C₁-C₁₀ alkyl-cycloalkyl, optionally substituted C₃-C₁₀ cycloalkyl, an optionally substituted branched C₁-C₇ haloalkyl, an optionally substituted linear C₁-C₇ haloalkyl, an optionally substituted C₁-C₇ alkylhydroxy, an optionally substituted C₁-C₇ alkoxy, a hydroxy, a heteroatom, a halogen, an alkyne, an alkoxy, an amino, or a combination thereof;
A represents a substituent comprising any one of an optionally substituted linear C₁-C₁₀ alkyl, an optionally substituted branched C₁-C₁₀ alkyl, an optionally substituted C₁-C₁₀ alkyl-aryl, an optionally substituted C₁-C₁₀ alkyl-cycloalkyl, an optionally substituted C₃-C₁₀ cycloalkyl, an optionally substituted branched C₁-C₇ haloalkyl, an optionally substituted linear C₁-C₇ haloalkyl, an optionally substituted C₁-C₇ alkylhydroxy, an optionally substituted unsaturated aliphatic ring, an optionally substituted C₃-C₁₀ heterocycle or a combination thereof;
[symbol] represents a single bond or a double bond; B is selected from O and OH; X, X₁ and X₂ are each independently selected from the group consisting of: S, O, N, and NH; and Y is selected from N and CH.

In some embodiments, R represents a substituent comprising any one of halogen, —C(═O)R′, —C(═O), —NO₂, —CN, —OR′, oxo, imino, —CONH₂, —CONR′₂, —CNNR′₂, —CSNR′₂, —CONH—OH, —CONH—NH₂, NHCOR′, —NHCSR′, —NHCNR′, —NC(═O)OR, —NC(═O)NR′, —NC(═S)OR′, —NC(═S)NR′, —SO₂R′, —SOR′, —SR′, —SO₂OR′, —SO₂N(R′)₂, —NHNR′₂, —NNR′, C₁-C₆ haloalkyl, optionally substituted C₁-C₆ alkyl, —NH₂, —N(R′)₂, —NR′₂—NH(C₁-C₆ alkyl), —N(C₁-C₆ alkyl)₂, C₁-C₆ alkoxy, C₁-C₆ haloalkoxy, hydroxy(C₁-C₆ alkyl), hydroxy(C₁-C₆ alkoxy), alkoxy(C₁-C₆ alkyl), alkoxy(C₁-C₆ alkoxy), C₁-C₆ alkyl-NR′₂, C₁-C₆ alkyl-SR′, —CONH(C₁-C₆ alkyl), —CON(C₁-C₆ alkyl)₂, —CO₂H, —CO₂R′, —OCOR, —OCOR′, —OC(═O)OR′, —OC(═O)NR′, —OC(═S)OR′, —OC(═S)NR′, a heteroatom, alkyne, an optionally substituted cycloalkyl, an optionally substituted heterocyclyl, an optionally substituted aryl or a combination thereof.

In one embodiment, provided herein is a composition comprising a compound of any one of Formulae I, Ia-lb and a pharmaceutically acceptable carrier. In one embodiment, provided herein a pharmaceutically acceptable salt of a compound of the invention such as but not limited to the compound of any one of Formulae I, Ia-Ib.

In some embodiments, the composition comprises the compound of the invention, a pharmaceutically acceptable salt thereof or both. In some embodiments, the composition is a pharmaceutical composition or a kit, comprising the compound of the invention (and/or any pharmaceutically acceptable salt, or any derivative thereof) and a pharmaceutically acceptable carrier. In some embodiments, the compound is a pharmaceutical grade compound, i.e., a compound characterized by a chemical purity of at least about 90%, at least about 95%, greater than 95%, or greater than 99%, and between 90 and 99.999%, between 90 and 95%, between 90 and 97%, between 95 and 99%, including any range in between.

In some embodiments, the compounds described herein are chiral compounds (i.e., possess an asymmetric carbon atom). In some embodiments, diastereomers, geometric isomers and individual isomers are encompassed within the scope of the present invention. In some embodiments, a chiral compound described herein is in the form of a racemic mixture. In some embodiments, a chiral compound is in the form of a single enantiomer, with an asymmetric carbon atom having the R configuration. In some embodiments, a chiral compound is in the form of a single enantiomer, with an asymmetric carbon atom having the S configuration as described hereinabove.

In some embodiments, a chiral compound is in the form of a single enantiomer with enantiomeric purity of more than 70%. In some embodiments, a chiral compound is in the form of a single enantiomer with enantiomeric purity of more than 80%. In some embodiments, a chiral compound is in a form of a single enantiomer with enantiomeric purity of more than 90%. In some embodiments, a chiral compound is in the form of a single enantiomer with enantiomeric purity of more than 95%.

In some embodiments, the compound of the invention comprising an unsaturated bond is in a form of a trans-, or cis-isomer. In some embodiments, the composition of the invention comprises a mixture of cis- and trans-isomers, as described hereinabove.

In some embodiments, the compounds described herein can exist in unsolvated form as well as in solvated form, including hydrated form. In general, the solvated form is equivalent to the unsolvated form and is encompassed within the scope of the present invention. Certain compounds of the present invention may exist in multiple crystalline or amorphous forms. In general, all physical forms are equivalent for the uses contemplated by the present invention and are intended to be within the scope of the present invention.

The term “solvate” refers to a complex of variable stoichiometry (e.g., di-, tri-, tetra-, penta-, hexa-, and so on), which is formed by a solute (the conjugate described herein) and a solvent, whereby the solvent does not interfere with the biological activity of the solute. Suitable solvents include, for example, ethanol, acetic acid and the like.

The term “hydrate” refers to a solvate, as defined hereinabove, where the solvent is water.

Unless otherwise indicated, structures depicted herein are also meant to include all isomeric (e.g., enantiomeric, diastereomeric, geometric, conformational, and rotational) forms of the structure. For example, the R and S configurations for each asymmetric center, (Z) and (E) double bond isomers, and (Z) and (E) conformational isomers are included in this invention. As would be understood to one skilled in the art, a substituent can freely rotate around any rotatable bond. Therefore, single stereochemical isomers as well as enantiomeric, diastereomeric, geometric, conformational, and rotational mixtures of the present compounds are within the scope of the invention.

Unless otherwise indicated, all tautomeric forms of the compounds of the invention are within the scope of the invention.

Additionally, unless otherwise indicated, structures depicted herein are also meant to include compounds that differ only in the presence of one or more isotopically enriched atoms. For example, compounds having the present structures except for the replacement of hydrogen by deuterium or tritium, or the replacement of a hydrogen by 18F, or the replacement of a carbon by a 13C- or 14C-enriched carbon are within the scope of this invention. Such compounds are useful, for example, as imaging probes.

In some embodiments, the compound of the invention includes any salt, any solvate, any hydrate, any stereoisomer, any isotope (e.g., a deuterated compound), and/or any derivative (e.g., a biologically active derivative) of any of the compounds or of the Formulae I, Ia-Ib disclosed herein.

In some embodiments, the compound or the composition of the present invention is the compound of any one of Formulae I, Ia-Ib, which inhibits tubulin polymerization and/or angiogenesis, and/or is characterized by an anti-cancer activity.

According to some embodiments, the present invention provides a compound or a composition (e.g. a pharmaceutical composition) as described herein, for use in the treatment of a disease or disorder associated with cancer. In some embodiments, the present invention provides a compound and/or a pharmaceutical composition of the invention for use in the treatment of an angiogenesis-associated disease or disorder.

According to some embodiments, the present invention provides a method for preventing or treating an angiogenesis-associated disease or disorder.

According to some embodiments, the present invention provides a method for preventing or treating a disease or disorder associated with cancer.

According to some embodiments, the present invention provides a method for treating or ameliorating the effects of a disease or disorder associated with cancer in a subject in need thereof comprising administering to the subject an effective amount of a compound or composition as described herein.

In some embodiments, the compound is represented by Formula Ia:

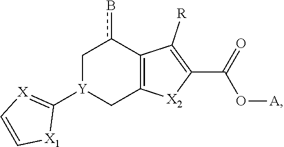

wherein R, A, B, Y, X, X₁ and X₂ are as described herein.

In some embodiments, the compound is represented by Formula II:

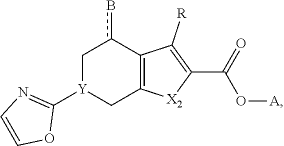

wherein A, B, Y, R and X₂ are as described herein.

In some embodiments, Y is selected from N and CH.

In some embodiments, X₂ is selected from O and NH.

In some embodiments, A comprises:

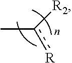

wherein n is 0 or an integer ranging from 1 to 10 (or from 1 to 2, from 2 to 5, from 5 to 10, including any range therebetween) each R is H or is as described herein, and wherein R₂ is H or represents a substituent comprising any one of halogen, —NO₂, —CN, —OR′, oxo, imino, —CONH₂, —CONR′₂, —CNNR′₂, —CSNR′₂, —CONH—OH, —CONH—NH₂, NHCOR′, —NHCSR′, —NHCNR′, —NC(═O)OR, —NC(═O)NR′, —NC(═S)OR′, —NC(═S)NR′, —SO₂R′, —SOR′, —SR′, —SO₂OR′, —SO₂N(R′)₂, —NHNR′₂, —NNR′, C₁-C₆ haloalkyl, optionally substituted C₁-C₆ alkyl, —NH₂, —N(R′)₂, —NR′₂—NH(C₁-C₆ alkyl), —N(C₁-C₆ alkyl)₂, C₁-C₆ alkoxy, C₁-C₆ haloalkoxy, hydroxy(C₁-C₆ alkyl), hydroxy(C₁-C₆ alkoxy), alkoxy(C₁-C₆ alkyl), alkoxy(C₁-C₆ alkoxy), C₁-C₆ alkyl-NR′₂, C₁-C₆ alkyl-SR′, —CONH(C₁-C₆ alkyl), —CON(C₁-C₆ alkyl)₂, —CO₂H, —CO₂R′, —OCOR, —OCOR′, —OC(═O)OR′, —OC(═O)NR′, —OC(═S)OR′, —OC(═S)NR′, a heteroatom, alkyne, an optionally substituted heterocyclyl, an optionally substituted aryl or a combination thereof; wherein R′ is as described hereinabove.

In some embodiments, n is 0. In some embodiments, n is between 2 and 5. In some embodiments, A comprises an optionally substituted cycloalkyl or heterocyclyl (e.g. C₃-C₈ cycloalkyl/heterocyclyl optionally substituted by one or more substituent, and/or optionally comprising one or more unsaturated bonds). In some embodiments, A is an alkyl/heteroalkyl comprising between 2 and 10, between 2 and 5, between 3 and 5, between 5 and 7 carbon atoms, including any range therebetween. In some embodiments, A is an alkyl/heteroalkyl being between 2 and 10, between 2 and 5, between 3 and 5, between 5 and 7 carbon atoms long, including any range therebetween. Each possibility represents a separate embodiment of the invention. In some embodiments, A comprises a linear or a branched C₂-C₁₀ alkyl/heteroalkyl, optionally substituted by one or more R (e.g. C₂-C₁₀ alkyl/heteroalkyl, or C₁-C₁₀ alkyl/heteroalkyl substituted by R at the terminal methylene). In some embodiments, A is selected from an optionally substituted cycloalkyl or heterocyclyl, C₁-C₁₀ alkyl/heteroalkyl, C₁-C₁₀ alkyl-amino, C₁-C₁₀ alkyl-mercapto, C₁-C₁₀ alkyl-hydroxy.

In some embodiments, the compound is selected from the group consisting of:

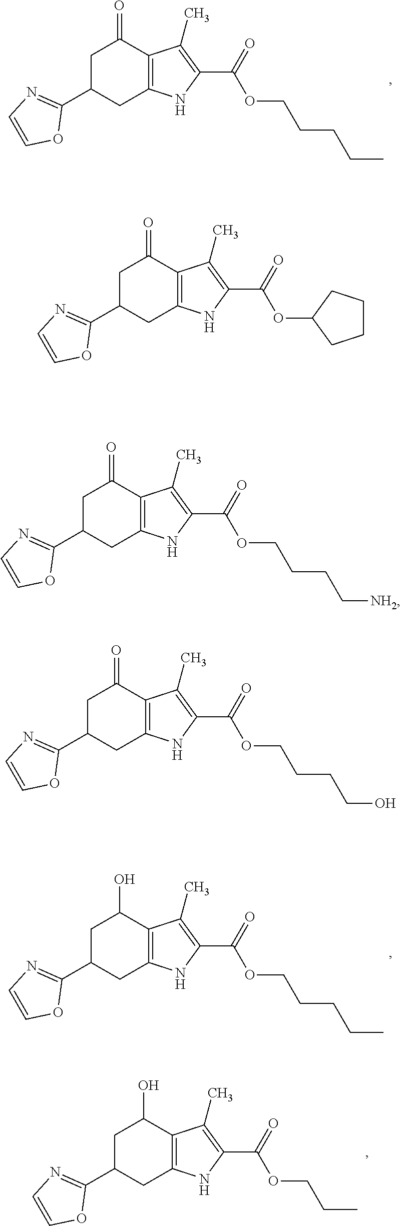 [SMILES: CCCCCOC(=O)c1[nH]c2c(c1C)C(=O)CC(c1ncco1)C2 ; Cc1c(C(=O)OC2CCCC2)[nH]c2c1C(=O)CC(c1ncco1)C2 ; Cc1c(C(=O)OCCCCN)[nH]c2c1C(=O)CC(c1ncco1)C2 ; Cc1c(C(=O)OCCCCO)[nH]c2c1C(=O)CC(c1ncco1)C2 ; CCCCCOC(=O)c1[nH]c2c(c1C)C(O)CC(c1ncco1)C2 ; CCCOC(=O)c1[nH]c2c(c1C)C(O)CC(c1ncco1)C2]

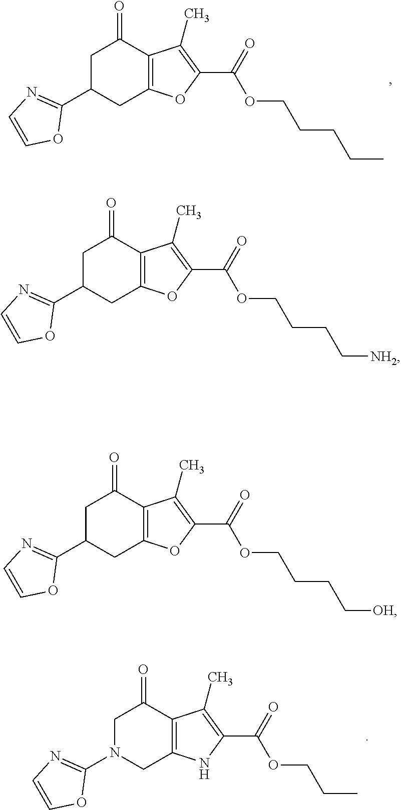 [SMILES: CCCCCOC(=O)c1oc2c(c1C)C(=O)CC(c1ncco1)C2 ; Cc1c(C(=O)OCCCCN)oc2c1C(=O)CC(c1ncco1)C2 ; Cc1c(C(=O)OCCCCO)oc2c1C(=O)CC(c1ncco1)C2 ; CCCOC(=O)c1[nH]c2c(c1C)C(=O)CN(c1ncco1)C2]

In some embodiments, the compound of the invention comprising oxazole ring is characterized by an increased aqueous solubility, compared to an analogous compound comprising furane or thiazole ring (as described in Example 1). In some embodiments, the compound of the invention is characterized by cytotoxic activity against cancer cell lines at low nanomolar levels (see example section, Example 2).

In some embodiments, the cancer cells line is selected from myeloma cells, acute lymphoblastic leukemia cells, acute monocytic leukemia cells, chronic myelogenous leukemia cells, pancreatic cancer cells, breast cancer cells, uterine sarcoma cells, cervix adenocarcinoma cells, ovary cancer cells, melanoma cells, osteosarcoma cells, prostate cancer cells, lung cancer cells, neuroblastoma cells, colorectal adenocarcinoma cells, or any combination thereof.

In some embodiments, the compound of the invention comprises any salt (e.g. a pharmaceutically acceptable salt), any tautomer, and/or any derivative or any stereoisomer thereof. In some embodiments, the compound as described hereinabove is the only active ingredient within the composition of the invention (e.g. pharmaceutical composition).

As used herein, the term “stereoisomer” refers to an enantiomer or to a diastereomer of the compound.

In some embodiments, the term “derivative” refers to a prodrug of the compound, such as ester; a tautomer of the compound, such as keto-enol tautomer, imine-enamine tautomer, amide-iminol tautomer, including any combination thereof. In some embodiments, the term “derivative” refers to a compound substituted by one or more (e.g. 2, 3, 4, or 5) substituents, optionally wherein any one of the substituents is selected from the group comprising (C₀-C₆)alkyl-aryl, (C₀-C₆)alkyl-heteroaryl, (C₀-C₆)alkyl-(C₃-C₈) cycloalkyl, optionally substituted C₃-C₈ heterocyclyl, halogen, —NO₂, —CN, —OH, —CONH₂, —CONR″₂, —CNNR″₂, —CSNR″₂, —CONH—OH, —CONH—NH₂, —NHCOR″, —NHCSR″, —NHCNR″, —NC(═O)OR″, —NC(═O)NR″, —NC(═S)OR″, —NC(═S)NR″, —SO₂R″, —SOR″, —SR″, —SO₂OR″, —SO₂N(R)₂, —NHNR₂, —NNR, C₁-C₁₀ haloalkyl, optionally substituted C₁-C₁₀ alkyl, —NH₂, —NH(C₁-C₁₀ alkyl), —N(C₁-C₁₀ alkyl)₂, C₁-C₁₀ alkoxy, C₁-C₁₀ haloalkoxy, hydroxy(C₁-C₁₀ alkyl), hydroxy(C₁-C₁₀ alkoxy), alkoxy(C₁-C₁₀ alkyl), alkoxy(C₁-C₁₀ alkoxy), C₁-C₁₀ alkyl-NR″₂, C₁-C₁₀ alkyl-SR, —CONH(C₁-C₁₀ alkyl), —CON(C₁-C₁₀ alkyl)₂, —CO₂H, —CO₂R″, —OCOR″, —OCOR″, —OC(═O)OR″, —OC(═O)NR″, —OC(═S)OR″, or —OC(═S)NR″, including any combination thereof, wherein R″ is selected from the group comprising an optionally substituted C₁-C₁₀ alkyl, an optionally substituted C₃-C₁₀ cycloalkyl, an optionally substituted C₃-C₁₀ heterocyclyl, an C₁-C₁₀ alkyl-aryl, an C₁-C₁₀ alkyl-cycloalkyl, an optionally substituted heteroaryl, an optionally substituted aryl, or a combination thereof.

As used herein, “R” and “Rn” with n representing any integer, refer to a substituent, wherein each R or Rn is as described herein. As used herein, the term “substituent” encompasses one or more substituents (e.g. 1, 2, 3, 4, or 5 substituents), wherein each of the substituents may be the same or different.

The term “substituted” encompasses a compound or a moiety, such as an alkyl, a cyclic ring, etc. having one or more substituent(s) covalently bound thereto. The term “substituent” independently encompasses any one of: —C(═O)R′, —C(═O), —OH, oxo, carbonyl, halogen, OR′, —NO2, —CN, —CONH2, —CONR′2, —CNNR′2, —CSNR′2, —CONH—OH, —CONH—NH2, —NHCOR′, —NHCSR′, —NHCNR′, —NC(═O)OR′, —NC(═O)NR′, —NC(═S)OR′, —NC(═S)NR′, —SO2R′, —SOR′, —SR′, —SO2OR′, —SO2N(R′)2, —NHNR′2, —NNR′, C₁-C₆ haloalkyl, optionally substituted C₁-C₆ alkyl, —NH2, —N(R′)2, —NH(C₁-C₆ alkyl), —N(C₁-C₆ alkyl)2, C₁-C₆ alkoxy, C₁-C₆ haloalkoxy, hydroxy(C₁-C₆ alkyl), hydroxy(C₁-C₆ alkoxy), alkoxy(C₁-C₆ alkyl), alkoxy(C₁-C₆ alkoxy), C₁-C₆ alkyl-NR′2, C₁-C₆ alkyl-SR′, —CONH(C₁-C₆ alkyl), —CON(C₁-C₆ alkyl)2, —CO2H, —CO2R′, —OCOR′, —OCOR′, —OC(═O)OR′, —OC(═O)NR′, —OC(═S)OR′, —OC(═S)NR′, optionally substituted aryl, optionally substituted heteroaryl, optionally substituted cycloalkyl, optionally substituted heterocyclic alkyl, or a combination thereof; wherein each R′ independently represents hydrogen, or is selected from the group comprising optionally substituted C₁-C₁₀ alkyl, optionally substituted C₁-C₃₀ alkyl, optionally substituted C₁-C₃₀ alkenyl, optionally substituted C₁-C₃₀ alkynyl, optionally substituted C₃-C₁₀ cycloalkyl, optionally substituted C₃-C₁₀ heterocyclyl, optionally substituted heteroaryl, optionally substituted aryl, hydroxy, amino, —NH₂, —NR′₂—NH(C₁-C₆ alkyl), —N(C₁-C₆ alkyl)₂, C₁-C₆ alkoxy, C₁-C₆haloalkoxy, hydroxy(C₁-C₆ alkyl), hydroxy(C₁-C₆ alkoxy), alkoxy(C₁-C₆ alkyl), alkoxy(C₁-C₆ alkoxy), C₁-C₆ alkyl-NR′₂, C₁-C₆ alkyl-SR′, or a combination thereof.

In some embodiments, the compound of the invention is represented by any of Formulae I, Ia-b and is characterized by water solubility of between 0.1 g/L and 10 g/L, between 0.1 g/L and 1 g/L, 0.5 g/L and 10 g/L, between 0.5 g/L and 1 g/L, between 1 g/L and 10 g/L, between 10 g/L and 30 g/L, between 5 g/L and 30 g/L, between 5 g/L and 20 g/L, between 30 g/L and 50 g/L, or between 50 g/L and 100 g/L, including any range between. Each possibility represents a separate embodiment of the present invention.

In some embodiments, a compound of the invention is a therapeutic agent. In some embodiments, the compound of the invention is an anti-cancer agent. In some embodiments, the compound of the invention is a microtubule targeting agent.

As used herein, the term “microtubule targeting agent” refers to any compound or drug that interferes in microtubule dynamics, for instance, by stabilization of microtubules or by destabilization of microtubules. Microtubule targeting drugs or microtubule targeting agents used in the clinic as anti-cancer drugs either stabilize or destabilize microtubules, affecting microtubule dynamics.

In some embodiments, a compound as described herein is a microtubule targeting agent and inhibits tubulin polymerization by inhibiting their interaction with the colchicine binding site of the tubulin protein.

In some embodiments, a compound as described herein inhibits tubulin polymerization by disrupting of mitotic spindle formation during cell division in rapidly dividing tumor cells, leading to mitotic arrest and subsequent apoptosis.

In some embodiments, a compound as described herein inhibits cancer cell migration.

### Method

According to some embodiments, the present invention provides a method for preventing or treating a disease in a subject, the method comprises administering a therapeutically effective amount of the compound of the invention (or of a pharmaceutical composition comprising the compound of the invention) to a subject in need thereof. In some embodiments, the disease or the disorder is cancer.

According to some embodiments, the present invention provides a method for treating or preventing a proliferative disease or disorder (e.g. cancer).

According to some embodiments, the present invention provides a method for treating or preventing development of cancer in a subject in need thereof, comprising administering to the subject the pharmaceutical composition described herein.

In some embodiments, the cancer is a metastatic cancer. In some embodiments, the cancer is selected from the group consisting of lung cancer, breast cancer, osteosarcoma, neuroblastoma, colon adenocarcinoma, chronic myelogenous leukemia (CML), acute lymphoblastic leukemia, acute monocytic leukemia, acute myeloid leukemia (AML), acute promyelocyte leukemia (APL), sarcoma, myxoma, rhabdomyoma, fibroma, lipoma, teratoma; bronchogenic carcinoma, alveolar carcinoma, bronchial adenoma, sarcoma, lymphoma, chondromatous hamartoma, mesothelioma, esophageal cancer, stomach cancer, pancreatic cancer, small bowel cancer, large bowel cancer; kidney cancer, bladder cancer, urethra cancer, prostate cancer, testis cancer; hepatoma, cholangiocarcinoma, hepatoblastoma, angiosarcoma, hepatocellular adenoma, hemangioma, osteogenic sarcoma, fibrosarcoma, malignant fibrous histiocytoma, chondrosarcoma, Ewing's sarcoma, malignant lymphoma, multiple myeloma, malignant giant cell tumor chordoma, osteochronfroma, benign chondroma, chondroblastoma, chondromyxofibroma, osteoid osteoma, giant cell tumors, cancer of the skuli, meninges cancer, brain cancer, spinal cord cancer, uterus cancer, cervical cancer, cancer of the ovaries, vulva cancer, vagina cancer, Hodgkin's disease, non-Hodgkin's lymphoma, malignant melanoma, basal cell carcinoma, squamous cell carcinoma, Karposi's sarcoma, moles dysplastic nevi, lipoma, angioma, uterine sarcoma, cervix adenocarcinoma, ovary cancer, colorectal adenocarcinoma and dermatofibroma.

According to some embodiments, the present invention provides a method for preventing or treating an angiogenesis-associated disease or disorder. According to some embodiments, the present invention provides a method for preventing or treating an angiogenesis-associated disease or disorder in a subject, comprising administering to the subject the pharmaceutical composition described herein, thereby preventing or treating an angiogenesis-associated disease or disorder in a subject.

In some embodiments, an angiogenesis related disease or disorder is selected from the group consisting of cancer, rheumatoid arthritis, diabetic retinopathy, retinopathy of prematurity, corneal graft rejection, neovascular glaucoma and retrolental fibroplasia, psoriasis, ocular neovascularization, endometriosis, epidemic keratoconjunctivitis, Vitamin A deficiency, contact lens overwear, atopic keratitis, superior limbic keratitis, pterygium keratitis sicca, sjogrens, acne rosacea, phylectenulosis, syphilis, Mycobacteria infections, lipid degeneration, chemical burns, bacterial ulcers, fungal ulcers, Herpes simplex infections, Herpes zoster infections, protozoan infections, Kaposi sarcoma, Mooren ulcer, Terrien's marginal degeneration, marginal keratolysis, trauma, rheumatoid arthritis, systemic lupus, polyarteritis, Wegener's sarcoidosis, scleritis, Stevens-Johnson disease, pemphigoid, radial keratotomy, corneal graph rejection, macular degeneration, sickle cell anemia, sarcoid, syphilis, pseudoxanthoma elasticum, Paget's disease, vein occlusion, artery occlusion, carotid obstructive disease, chronic uveitis/vitreitis, mycobacterial infections, Lyme's disease, systemic lupus erythematosus, retinopathy of prematurity, Eales' disease, Behcet's disease, infections causing a retinitis or choroiditis, presumed ocular histoplasmosis, Best's disease, myopia, optic pits, Stargart's disease, pars planitis, chronic retinal detachment, hyperviscosity syndromes, toxoplasmosis, trauma and post-laser complications.

In some embodiments, the method is for preventing or treating a disease or disorder associated with the efflux pump protein, P-glycoprotein (Pgp) overexpression. In some embodiments, overexpression refers to an enhanced concentration of Pgp within a tissue of the subject, wherein enhanced is by at least 10% compared to a normal expression (e.g. concentration of Pgp within a tissue of a healthy subject).

In some embodiments, the method is for inhibiting tubulin polymerization.

According to some embodiments, the present invention provides a method preventing or treating a disease or disorder associated with tubulin polymerization in a subject, comprising administering to the subject the pharmaceutical composition described herein, thereby preventing or treating a disease or disorder associated with tubulin polymerization in a subject.

In some embodiments, the administering is by an oral administration, a topical administration, a systemic administration or any combination thereof.

### Pharmaceutical Composition

In another aspect of the invention disclosed herein, there is a pharmaceutical composition comprising the compound of the invention, a pharmaceutically acceptable salt thereof or both. In some embodiments, the pharmaceutical composition of the invention comprises a therapeutically effective amount of the compound of the invention and/or any pharmaceutically acceptable salt and/or derivative thereof. In some embodiments, therapeutically effective amount is sufficient for reduction of at least one symptom, or for substantial reduction in the severity and/or inhibition of the progression of a disease, disorder, or condition as described hereinabove. In some embodiments, the therapeutically effective amount can be determined as described hereinabove.

In some embodiments, there is provided herein a composition comprising one or more compounds of the invention, including any salt (e.g. a pharmaceutically acceptable salt), any tautomer, and/or any stereoisomer thereof. In some embodiments, the compound as described hereinabove is the only active ingredient within the composition of the invention (e.g. pharmaceutical composition).

In some embodiments, the composition of the invention is a pharmaceutical composition comprising at least one compound of the invention and a pharmaceutically acceptable carrier. In some embodiments, the composition of the invention is a pharmaceutical composition comprising at least one compound of the invention as a first active ingredient and an additional active ingredient.

Non-limiting examples of pharmaceutically acceptable salts include but are not limited to: acetate, aspartate, benzenesulfonate, benzoate, bicarbonate, carbonate, halide (such as bromide, chloride, iodide, fluoride), bitartrate, citrate, salicylate, stearate, succinate, sulfate, tartrate, decanoate, edetate, fumarate, gluconate, and lactate or any combination thereof.

In some embodiments, the pharmaceutical composition comprises the compound of the invention and a pharmaceutically acceptable carrier. In some embodiments, the pharmaceutical composition comprises a therapeutically effective amount of the compound of the invention and the pharmaceutically acceptable carrier.

In some embodiments, the pharmaceutical composition is in a form of a combination or of a kit of parts. In some embodiments, the pharmaceutical composition of the invention is for use as a medicament.

For example, the term “pharmaceutically acceptable” can mean approved by a regulatory agency of the Federal or a state government or listed in the U.S. Pharmacopeia or other generally recognized pharmacopeia for use in animals, and more particularly in humans. In some embodiments, the compound of the invention is referred to herein as an active ingredient of a pharmaceutical composition.

In some embodiments, the pharmaceutical composition as described herein is a topical composition. In some embodiments, the pharmaceutical composition is an oral composition. In some embodiments, the pharmaceutical composition is an injectable composition. In some embodiments, the pharmaceutical composition is for a systemic use.

As used herein, the term “carrier” refers to a diluent, adjuvant, excipient, or vehicle with which the active ingredient is administered. Such carriers can be sterile liquids, such as water-based and oils, including those of petroleum, animal, vegetable or synthetic origin, such as peanut oil, soybean oil, mineral oil, sesame oil and the like, polyethylene glycols, glycerin, propylene glycol or other synthetic solvents.

Other non-limiting examples of carriers include, but are not limited to: terpenes derived from Cannabis, or total terpene extract from Cannabis plants, terpenes from coffee or cocoa, mint-extract, eucalyptus-extract, citrus-extract, tobacco-extract, anis-extract, any vegetable oil, peppermint oil, d-limonene, b-myrcene, a-pinene, linalool, anethole, a-bisabolol, camphor, b-caryophyllene and caryophyllene oxide, 1,8-cineole, citral, citronella, delta-3-carene, farnesol, geraniol, indomethacin, isopulegol, linalool, unalyl acetate, b-myrcene, myrcenol, 1-menthol, menthone, menthol and neomenthol, oridonin, a-pinene, diclofenac, nepafenac, bromfenac, phytol, terpineol, terpinen-4-ol, thymol, and thymoquinone. One skilled in the art will appreciate, that a particular carrier used within the pharmaceutical composition of the invention may vary depending on the route of administration.

In some embodiments, the carrier improves the stability of the active ingredient in a living organism. In some embodiments, the carrier improves the stability of the active ingredient within the pharmaceutical composition. In some embodiments, the carrier enhances the bioavailability of the active ingredient.

Water may be used as a carrier such as when the active ingredient has a sufficient aqueous solubility, so as to be administered intravenously. Saline solutions and aqueous dextrose and glycerol solutions can also be employed as liquid carriers, particularly for injectable solutions.

In some embodiments, the carrier is a liquid carrier. In some embodiments, the carrier is an aqueous carrier.

Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene glycol, water, ethanol and the like. The composition, if desired, can also contain minor amounts of wetting or emulsifying agents, or pH buffering agents such as acetates, citrates or phosphates. Antibacterial agents such as benzyl alcohol or methyl parabens; antioxidants such as ascorbic acid or sodium bisulfite; and agents for the adjustment of tonicity such as sodium chloride or dextrose are also envisioned. The carrier may comprise, in total, from 0.1% to 99.99999% by weight of the composition/s or the pharmaceutical composition/s presented herein.

In some embodiments, the pharmaceutical composition includes incorporation of any one of the active ingredients into or onto particulate preparations of polymeric compounds such as polylactic acid, polyglycolic acid, hydrogels, etc., or onto liposomes, microemulsions, micelles, unilamellar or multilamellar vesicles, erythrocyte ghosts, or spheroplasts. Such compositions may influence the physical state, solubility, stability, rate of in vivo release, and rate of in vivo clearance.

In some embodiments, the pharmaceutical composition comprising the compound of the invention is in a unit dosage form. In some embodiments, the pharmaceutical composition is prepared by any of the methods well known in the art of pharmacy. In some embodiments, the unit dosage form is in the form of a tablet, capsule, lozenge, wafer, patch, ampoule, vial or pre-filled syringe.

In addition, in vitro assays may optionally be employed to help identify optimal dosage ranges. The precise dose to be employed in the formulation will also depend on the route of administration, and the nature of the disease or disorder, and should be decided according to the judgment of the practitioner and each patient's circumstances. Effective doses can be extrapolated from dose-response curves derived from in-vitro or in-vivo animal model test bioassays or systems. In some embodiments, the effective dose is determined as described hereinabove.

In another embodiment, the pharmaceutical composition of the invention is administered in any conventional oral, parenteral or transdermal dosage form.

As used herein, the terms “administering”, “administration”, and like terms refer to any method which, in sound medical practice, delivers a composition containing an active agent to a subject in such a manner as to provide a therapeutic effect. In some embodiments, administering is by an oral administration, a systemic administration or a combination thereof.

In some embodiments, the pharmaceutical composition is administered via oral (i.e., enteral), rectal, vaginal, topical, nasal, ophthalmic, transdermal, subcutaneous, intramuscular, intraperitoneal or intravenous routes of administration. The route of administration of the pharmaceutical composition will depend on the disease or condition to be treated. Suitable routes of administration include, but are not limited to, parenteral injections, e.g., intradermal, intravenous, intramuscular, intralesional, subcutaneous, intrathecal, and any other mode of injection as known in the art. In addition, it may be desirable to introduce the pharmaceutical composition of the invention by any suitable route, including intraventricular and intrathecal injection; intraventricular injection may be facilitated by an intraventricular catheter, for example, attached to a reservoir. Pulmonary administration can also be employed, e.g., by use of an inhaler or nebulizer.

In some embodiments, for oral applications, the pharmaceutical composition or is in the form of a tablets or a capsule, which can contain any of the following ingredients, or compounds of a similar nature: a binder such as microcrystalline cellulose, gum tragacanth or gelatin; an excipient such as starch or lactose; a disintegrating agent such as alginic acid, Primogel, or corn starch; a lubricant such as magnesium stearate; or a glidant such as colloidal silicon dioxide. When the dosage unit form is a capsule, it can contain, in addition to materials of the above type, a liquid carrier such as fatty oil. In addition, dosage unit forms can contain various other materials which modify the physical form of the dosage unit, for example, coatings of sugar, shellac, or other enteric agents. In some embodiments, the tablet of the invention is further film coated. In some embodiments, oral application of the pharmaceutical composition or of the kit is in a form of a drinkable liquid. In some embodiments, oral application of the pharmaceutical composition or of the kit is in a form of an edible product.

For purposes of parenteral administration, solutions in sesame or peanut oil or in aqueous propylene glycol can be employed, as well as sterile aqueous solutions of the corresponding water-soluble salts. Such aqueous solutions may be suitably buffered, if necessary, and the liquid diluent first rendered isotonic with sufficient saline or glucose. These aqueous solutions are especially suitable for intravenous, intramuscular, subcutaneous and intraperitoneal injection purposes.

In some embodiments, the pharmaceutical composition is for use in the inhibition of a P glycoprotein mediated multi-drug resistant cancer. In some embodiments, the pharmaceutical composition is for use in the prevention or treatment of an angiogenesis-associated disease or disorder. In some embodiments, the pharmaceutical composition is for use in the prevention or treatment of a disease or disorder associated with cancer.

In some embodiments, the pharmaceutical composition is for use in disrupting microtubule polymerization in a cell.

### Pharmaceutically Acceptable Salts

In some embodiments, the compounds of the present invention can exist in free form for treatment, or as a pharmaceutically acceptable salt.

As used herein, the term “pharmaceutically acceptable salt” refers to any non-toxic salt of a compound of the present invention that, upon administration to a subject, e.g., a human, is capable of providing, either directly or indirectly, a compound of this invention or an inhibitory active metabolite or residue thereof. For example, the term “pharmaceutically acceptable” can mean approved by a regulatory agency of the Federal or a state government or listed in the U. S. Pharmacopeia or other generally recognized pharmacopeia for use in animals, and more particularly in humans.

As used herein, the term “carrier” refers to a diluent, adjuvant, excipient, or vehicle with which the therapeutic compound is administered. Such pharmaceutical carriers can be sterile liquids, such as water and oils, including those of petroleum, animal, vegetable or synthetic origin, such as peanut oil, soybean oil, mineral oil, sesame oil and the like, polyethylene glycols, glycerin, propylene glycol or other synthetic solvents. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene glycol, water, ethanol and the like. The composition, if desired, can also contain minor amounts of wetting or emulsifying agents, or pH buffering agents such as acetates, citrates or phosphates. Antibacterial agents such as benzyl alcohol or methyl parabens; antioxidants such as ascorbic acid or sodium bisulfite; and agents for the adjustment of tonicity such as sodium chloride or dextrose are also envisioned. The carrier may comprise, in total, from about 0.1% to about 99.99999% by weight of the pharmaceutical compositions presented herein.

Pharmaceutically acceptable salts are well known in the art. For example, S. M. Berge et al., describe pharmaceutically acceptable salts in detail in J. Pharmaceutical Sciences, 1977, 66, 1-19. Pharmaceutically acceptable salts of the compounds of this invention include those derived from suitable inorganic and organic acids and bases. These salts can be prepared in situ during the final isolation and purification of the compounds. Acid addition salts can be prepared by 1) reacting the purified compound in its free-based form with a suitable organic or inorganic acid and 2) isolating the salt thus formed.

Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, glycolate, gluconate, glycolate, hemisulfate, heptanoate, hexanoate, hydrochloride, hydrobromide, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, palmoate, pectinate, persulfate, 3-phenylpropionate, phosphate, picrate, pivalate, propionate, salicylate, stearate, succinate, sulfate, tartrate, thiocyanate, p-toluenesulfonate, undecanoate, valerate salts, and the like.

Base addition salts can be prepared by 1) reacting the purified compound in its acid form with a suitable organic or inorganic base and 2) isolating the salt thus formed. Salts derived from appropriate bases include alkali metal (e.g., sodium, lithium, and potassium), alkaline earth metal (e.g., magnesium and calcium), ammonium and N+(C₁-4alkyl)4 salts. This invention also envisions the quaternization of any basic nitrogen-containing groups of the compounds disclosed herein. Water or oil-soluble or dispersible products may be obtained by such quaternization.

Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate. Other acids and bases, while not in themselves pharmaceutically acceptable, may be employed in the preparation of salts useful as intermediates in obtaining the compounds of the invention and their pharmaceutically acceptable acid or base addition salts.

In some embodiments, the compounds described herein are chiral compounds (i.e. possess an asymmetric carbon atom). In some embodiments, diastereomers, geometric isomers and individual isomers are encompassed within the scope of the present invention. In some embodiments, a chiral compound described herein is in form of a racemic mixture. In some embodiments, a chiral compound is in form of a single enantiomer, with an asymmetric carbon atom having the R configuration. In some embodiments, a chiral compound is in form of a single enantiomer, with an asymmetric carbon atom having the S configuration as described hereinabove.

In some embodiments, a chiral compound is in form of a single enantiomer with enantiomeric purity of more than 70%. In some embodiments, a chiral compound is in form of a single enantiomer with enantiomeric purity of more than 80%. In some embodiments, a chiral compound is in form of a single enantiomer with enantiomeric purity of more than 90%. In some embodiments, a chiral compound is in form of a single enantiomer with enantiomeric purity of more than 95%.

In some embodiments, the compound of the invention comprising an unsaturated bond is in a form of a trans-, or cis-isomer. In some embodiments, the composition of the invention comprises a mixture of cis- and trans-isomers, as described hereinabove.

In some embodiments, the compounds described herein can exist in unsolvated form as well as in solvated form, including hydrated form. In general, the solvated form is equivalent to the unsolvated form and is encompassed within the scope of the present invention. Certain compounds of the present invention may exist in multiple crystalline or amorphous forms. In general, all physical forms are equivalent for the uses contemplated by the present invention and are intended to be within the scope of the present invention.

The term “solvate” refers to a complex of variable stoichiometry (e.g., di-, tri-, tetra-, penta-, hexa-, and so on), which is formed by a solute (the conjugate described herein) and a solvent, whereby the solvent does not interfere with the biological activity of the solute. Suitable solvents include, for example, ethanol, acetic acid and the like.

The term “hydrate” refers to a solvate, as defined hereinabove, where the solvent is water.

Unless otherwise indicated, structures depicted herein are also meant to include all isomeric (e.g., enantiomeric, diastereomeric, geometric, conformational, and rotational) forms of the structure. For example, the R and S configurations for each asymmetric center, (Z) and (E) double bond isomers, and (Z) and (E) conformational isomers are included in this invention. As would be understood to one skilled in the art, a substituent can freely rotate around any rotatable bonds. Therefore, single stereochemical isomers as well as enantiomeric, diastereomeric, geometric, conformational, and rotational mixtures of the present compounds are within the scope of the invention.

Unless otherwise indicated, all tautomeric forms of the compounds of the invention are within the scope of the invention.

Additionally, unless otherwise indicated, structures depicted herein are also meant to include compounds that differ only in the presence of one or more isotopically enriched atoms. For example, compounds having the present structures except for the replacement of hydrogen by deuterium or tritium, or the replacement of a carbon by a ¹³C- or ¹⁴C-enriched carbon are within the scope of this invention. Such compounds are useful, for example, as analytical tools or probes in biological assays.

In another embodiment, the compositions of the invention take the form of solutions, suspensions, emulsions, tablets, pills, capsules, powders, gels, creams, ointments, foams, pastes, sustained-release formulations and the like. In another embodiment, the compositions of the invention can be formulated as a suppository, with traditional binders and carriers such as triglycerides, microcrystalline cellulose, gum tragacanth or gelatin. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in: Remington's Pharmaceutical Sciences” by E.W. Martin, the contents of which are hereby incorporated by reference herein. Such compositions will contain a therapeutically effective amount of the polypeptide of the invention, preferably in a substantially purified form, together with a suitable amount of carrier so as to provide the form for proper administration to the subject.

### Definitions

As used herein, the term “alkyl” describes an aliphatic hydrocarbon including straight chain and branched chain groups. The term “alkyl”, as used herein, also encompasses saturated or unsaturated hydrocarbon, hence this term further encompasses alkenyl and alkynyl.

The term “alkenyl” describes an unsaturated alkyl, as defined herein, having at least two carbon atoms and at least one carbon-carbon double bond. The alkenyl may be substituted or unsubstituted by one or more substituents, as described hereinabove.

The term “alkynyl”, as defined herein, is an unsaturated alkyl having at least two carbon atoms and at least one carbon-carbon triple bond. The alkynyl may be substituted or unsubstituted by one or more substituents, as described hereinabove.

The term “cycloalkyl” describes an all-carbon monocyclic or fused ring (i.e. rings which share an adjacent pair of carbon atoms) group where one or more of the rings does not have a completely conjugated pi-electron system. The cycloalkyl group may be substituted or unsubstituted, as indicated herein.

The term “aryl” describes an all-carbon monocyclic or fused-ring polycyclic (i.e. rings which share adjacent pairs of carbon atoms) groups having a completely conjugated pi-electron system. The aryl group may be substituted or unsubstituted, as indicated herein.

The term “alkoxy” describes both an O-alkyl and an —O-cycloalkyl group, as defined herein. The term “aryloxy” describes an —O-aryl, as defined herein.

Each of the alkyl, cycloalkyl and aryl groups in the general formulas herein may be substituted by one or more substituents, whereby each substituent group can independently be, for example, halide, alkyl, alkoxy, cycloalkyl, nitro, amino, hydroxyl, thiol, thioalkoxy, carboxy, amide, aryl and aryloxy, depending on the substituted group and its position in the molecule. Additional substituents are also contemplated.

The term “halide”, “halogen” or “halo” describes fluorine, chlorine, bromine or iodine. The term “haloalkyl” describes an alkyl group as defined herein, further substituted by one or more halide(s). The term “haloalkoxy” describes an alkoxy group as defined herein, further substituted by one or more halide(s). The term “hydroxyl” or “hydroxy” describes a —OH group. The term “mercapto” or “thiol” describes a —SH group. The term “thioalkoxy” describes both an —S-alkyl group, and a —S-cycloalkyl group, as defined herein. The term “thioaryloxy” describes both an —S-aryl and a —S-heteroaryl group, as defined herein. The term “amino” describes a —NR′R″ group, or a salt thereof, with R′ and R″ as described herein.

The term “heterocyclyl” describes a monocyclic or fused ring group having in the ring(s) one or more atoms such as nitrogen, oxygen and sulfur. The rings may also have one or more double bonds. However, the rings do not have a completely conjugated pi-electron system. Representative examples are piperidine, piperazine, tetrahydrofuran, tetrahydropyran, morpholino and the like.

The term “carboxy” describes a —C(O)OR′ group, or a carboxylate salt thereof, where R′ is hydrogen, alkyl, cycloalkyl, alkenyl, aryl, heteroaryl (bonded through a ring carbon) or heterocyclyl (bonded through a ring carbon) as defined herein. or “carboxylate”

The term “carbonyl” describes a —C(O)R′ group, where R′ is as defined hereinabove. The above-terms also encompass thio-derivatives thereof (thiocarboxy and thiocarbonyl).

The term “thiocarbonyl” describes a —C(S)R′ group, where R′ is as defined hereinabove. A “thiocarboxy” group describes a —C(S)OR′ group, where R′ is as defined herein. A “sulfinyl” group describes an —S(O)R′ group, where R′ is as defined herein. A “sulfonyl” or “sulfonate” group describes an —S(O)2R′ group, where R′ is as defined herein.

A “carbamyl” or “carbamate” group describes an —OC(O)NR′R″ group, where R′ is as defined herein and R″ is as defined for R′. A “nitro” group refers to a —NO2 group. The term “amide” as used herein encompasses C-amide and N-amide. The term “C-amide” describes a —C(O)NR′R″ end group or a —C(O)NR′-linking group, as these phrases are defined hereinabove, where R′ and R″ are as defined herein. The term “N-amide” describes a —NR″C(O)R′ end group or a —NR′C(O)— linking group, as these phrases are defined hereinabove, where R′ and R″ are as defined herein.

A “cyano” or “nitrile” group refers to a —CN group. The term “azo” or “diazo” describes an —N═NR′ end group or an —N═N— linking group, as these phrases are defined hereinabove, with R′ as defined hereinabove. The term “guanidine” describes a —R′NC(N)NR″R′″ end group or a —R′NC(N)NR″— linking group, as these phrases are defined hereinabove, where R′, R″ and R′″ are as defined herein. As used herein, the term “azide” refers to a —N3 group. The term “sulfonamide” refers to a —S(O)2NR′R″ group, with R′ and R″ as defined herein.

The term “phosphonyl” or “phosphonate” describes an —OP(O)—(OR′)2 group, with R′ as defined hereinabove. The term “phosphinyl” describes a —PR′R″ group, with R′ and R″ as defined hereinabove. The term “alkylaryl” describes an alkyl, as defined herein, which substituted by an aryl, as described herein. An exemplary alkylaryl is benzyl.

The term “heteroaryl” describes a monocyclic or fused ring (i.e. rings which share an adjacent pair of atoms) group having in the ring(s) one or more atoms, such as, for example, nitrogen, oxygen and sulfur and, in addition, having a completely conjugated pi-electron system. As used herein, the term “heteroaryl” refers to an aromatic ring in which at least one atom forming the aromatic ring is a heteroatom. Heteroaryl rings can be foamed by three, four, five, six, seven, eight, nine and more than nine atoms. Heteroaryl groups can be optionally substituted. Examples of heteroaryl groups include, but are not limited to, aromatic C₃₋₈heterocyclic groups containing one oxygen or sulfur atom, or two oxygen atoms, or two sulfur atoms or up to four nitrogen atoms, or a combination of one oxygen or sulfur atom and up to two nitrogen atoms, and their substituted as well as benzo- and pyrido-fused derivatives, for example, connected via one of the ring-forming carbon atoms. In certain embodiments, heteroaryl is selected from among oxazolyl, isoxazolyl, oxadiazolyl, thiazolyl, isothiazolyl, pyridinyl, pyridazinyl, pyrimidinal, pyrazinyl, indolyl, benzimidazolyl, quinolinyl, isoquinolinyl, quinazolinyl or quinoxalinyl.

In some embodiments, a heteroaryl group is selected from among pyrrolyl, furanyl (furyl), thiophenyl (thienyl), imidazolyl, pyrazolyl, 1,2,3-triazolyl, 1,2,4-triazolyl, 1,3-oxazolyl (oxazolyl), 1,2-oxazolyl (isoxazolyl), oxadiazolyl, 1,3-thiazolyl (thiazolyl), 1,2-thiazolyl (isothiazolyl), tetrazolyl, pyridinyl (pyridyl)pyridazinyl, pyrimidinyl, pyrazinyl, 1,2,3-triazinyl, 1,2,4-triazinyl, 1,3,5-triazinyl, 1,2,4,5-tetrazinyl, indazolyl, indolyl, benzothiophenyl, benzofuranyl, benzothiazolyl, benzimidazolyl, benzodioxolyl, acridinyl, quinolinyl, isoquinolinyl, quinazolinyl, quinoxalinyl, phthalazinyl, thienothiophenyl, 1,8-naphthyridinyl, other naphthyridinyls, pteridinyl or phenothiazinyl. Where the heteroaryl group includes more than one ring, each additional ring is the saturated form (perhydro form) or the partially unsaturated form (e.g., the dihydro form or tetrahydro form) or the maximally unsaturated (nonaromatic) form. The term heteroaryl thus includes bicyclic radicals in which the two rings are aromatic and bicyclic radicals in which only one ring is aromatic. Such examples of heteroaryl are include 3H-indolinyl, 2(1H)-quinolinonyl, 4-oxo-1,4-dihydroquinolinyl, 2H-1-oxoisoquinolyl, 1,2-dihydroquinolinyl, (2H)quinolinyl N-oxide, 3,4-dihydroquinolinyl, 1,2-dihydroisoquinolinyl, 3,4-dihydro-isoquinolinyl, chromonyl, 3,4-dihydroiso-quinoxalinyl, 4-(3H)quinazolinonyl, 4H-chromenyl, 4-chromanonyl, oxindolyl, 1,2,3,4-tetrahydroisoquinolinyl, 1,2,3,4-tetrahydro-quinolinyl, 1H-2,3-dihydroisoindolyl, 2,3-dihydrobenzo[f]isoindolyl, 1,2,3,4-tetrahydrobenzo-[g]isoquinolinyl, 1,2,3,4-tetrahydro-benzo[g]isoquinolinyl, chromanyl, isochromanonyl, 2,3-dihydrochromonyl, 1,4-benzo-dioxanyl, 1,2,3,4-tetrahydro-quinoxalinyl, 5,6-dihydro-quinolyl, 5,6-dihydroiso-quinolyl, 5,6-dihydroquinoxalinyl, 5,6-dihydroquinazolinyl, 4,5-dihydro-1H-benzimidazolyl, 4,5-dihydro-benzoxazolyl, 1,4-naphthoquinolyl, 5,6,7,8-tetrahydro-quinolinyl, 5,6,7,8-tetrahydro-isoquinolyl, 5,6,7,8-tetrahydroquinoxalinyl, 5,6,7,8-tetrahydroquinazolyl, 4,5,6,7-tetrahydro-1H-benzimidazolyl, 4,5,6,7-tetrahydro-benzoxazolyl, 1H-4-oxa-1,5-diaza-naphthalen-2-onyl, 1,3-dihydroimidizolo-[4,5]-pyridin-2-onyl, 2,3-dihydro-1,4-dinaphtho-quinonyl, 2,3-dihydro-1H-pyrrol[3,4-b]quinolinyl, 1,2,3,4-tetrahydrobenzo[b]-[1,7]naphthyridinyl, 1,2,3,4-tetra-hydrobenz[b][1,6]-naphthyridinyl, 1,2,3,4-tetrahydro-9H-pyrido[3,4-b]indolyl, 1,2,3,4-tetrahydro-9H-pyrido[4,3-b]indolyl, 2,3-dihydro-1H-pyrrolo-[3,4-b]indolyl, 1H-2,3,4,5-tetrahydro-azepino[3,4-b]indolyl, 1H-2,3,4,5-tetrahydroazepino-[4,3-b]indolyl, 1H-2,3,4,5-tetrahydro-azepino[4,5-b]indolyl, 5,6,7,8-tetrahydro[1,7]napthyridinyl, 1,2,3,4-tetrahydro-[2,7]-naphthyridyl, 2,3-dihydro[1,4]dioxino[2,3-b]pyridyl, 2,3-dihydro[1,4]-dioxino[2,3-b]pryidyl, 3,4-dihydro-2H-1-oxa[4,6]diazanaphthalenyl, 4,5,6,7-tetrahydro-3H-imidazo-[4,5-c]pyridyl, 6,7-dihydro[5,8]diazanaphthalenyl, 1,2,3,4-tetrahydro[1,5]-napthyridinyl, 1,2,3,4-tetrahydro[1,6]napthyridinyl, 1,2,3,4-tetrahydro[1,7]napthyridinyl, 1,2,3,4-tetrahydro-[1,8]napthyridinyl or 1,2,3,4-tetrahydro[2,6]napthyridinyl. In some embodiments, heteroaryl groups are optionally substituted. In one embodiment, the one or more substituents are each independently selected from among halo, hydroxy, amino, cyano, nitro, alkylamido, acyl, C₁₋₆-alkyl, C₁₋₆-haloalkyl, C₁₋₆-hydroxyalkyl, C₁₋₆-aminoalkyl, C₁₋₆-alkylamino, alkylsulfenyl, alkylsulfinyl, alkylsulfonyl, sulfamoyl, or trifluoromethyl.

Examples of heteroaryl groups include, but are not limited to, unsubstituted and mono- or di-substituted derivatives of furan, benzofuran, thiophene, benzothiophene, pyrrole, pyridine, indole, oxazole, benzoxazole, isoxazole, benzisoxazole, thiazole, benzothiazole, isothiazole, imidazole, benzimidazole, pyrazole, indazole, tetrazole, quinoline, isoquinoline, pyridazine, pyrimidine, purine and pyrazine, furazan, 1,2,3-oxadiazole, 1,2,3-thiadiazole, 1,2,4-thiadiazole, triazole, benzotriazole, pteridine, phenoxazole, oxadiazole, benzopyrazole, quinolizine, cinnoline, phthalazine, quinazoline and quinoxaline. In some embodiments, the substituents are halo, hydroxy, cyano, O—C₁₋₆-alkyl, C₁₋₆-alkyl, hydroxy-C₁₋₆-alkyl and amino-C₁₋₆-alkyl.

As used herein, the terms “halo” and “halide”, which are referred to herein interchangeably, describe an atom of a halogen, that is fluorine, chlorine, bromine or iodine, also referred to herein as fluoride, chloride, bromide and iodide.

### General

As used herein the term “about” refers to ±10%.

The terms “comprises”, “comprising”, “includes”, “including”, “having” and their conjugates mean “including but not limited to”.

The term “consisting of means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

Generally, the nomenclature used herein, and the laboratory procedures utilized in the present invention include molecular, biochemical, and microbiological techniques. Such techniques are thoroughly explained in the literature.

### Materials and Methods

### Synthetic Procedures

An exemplary synthetic procedure for preparation of pentyl 3-methyl-6-(oxazol-2-yl)-4-oxo-4,5,6,7-tetrahydro-1H-indole-2-carboxylate (SMS077) is as follows:

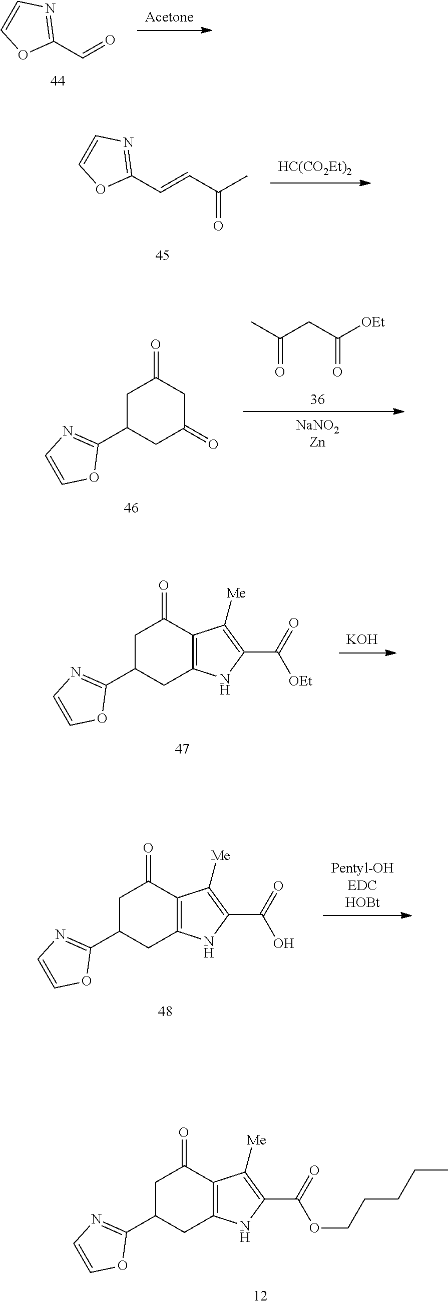

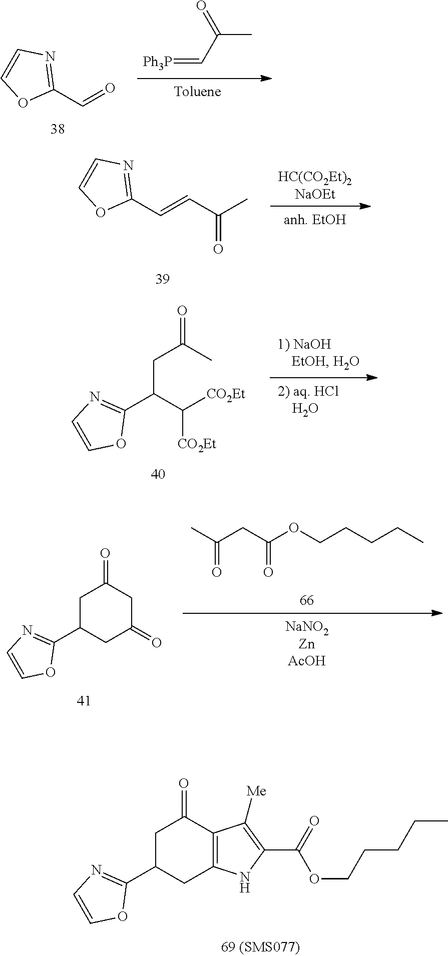

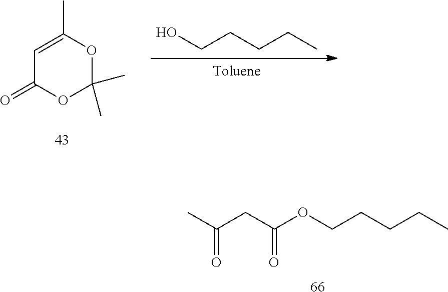

An exemplary synthetic procedure for preparation of pentyl-3-methyl-6-(oxazol-2-yl)-4-oxo-4,5,6,7-tetrahydrobenzofuran-2-carboxylate (SMS182) is as follows:

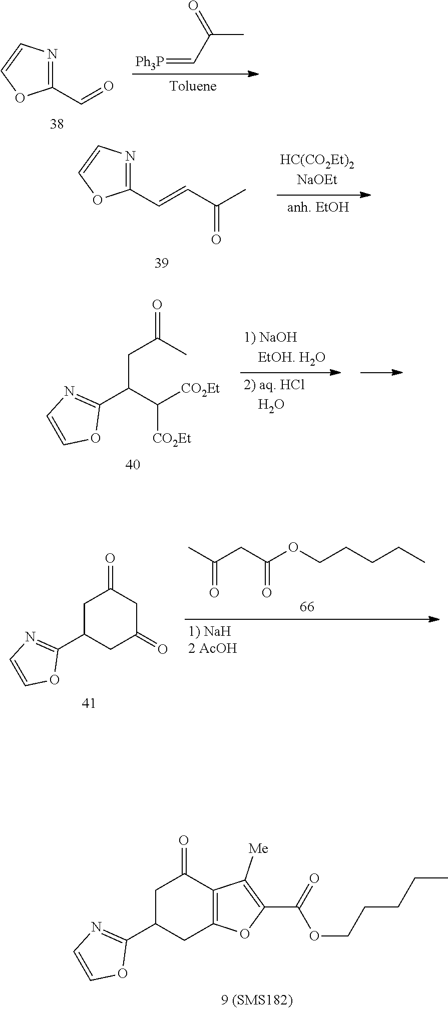

An exemplary synthetic procedure for preparation of cyclopentyl 3-methyl-6-(oxazol-2-yl)-4-oxo-4,5,6,7-tetrahydro-1H-indole-2-carboxylate (SMS101) is as follows:

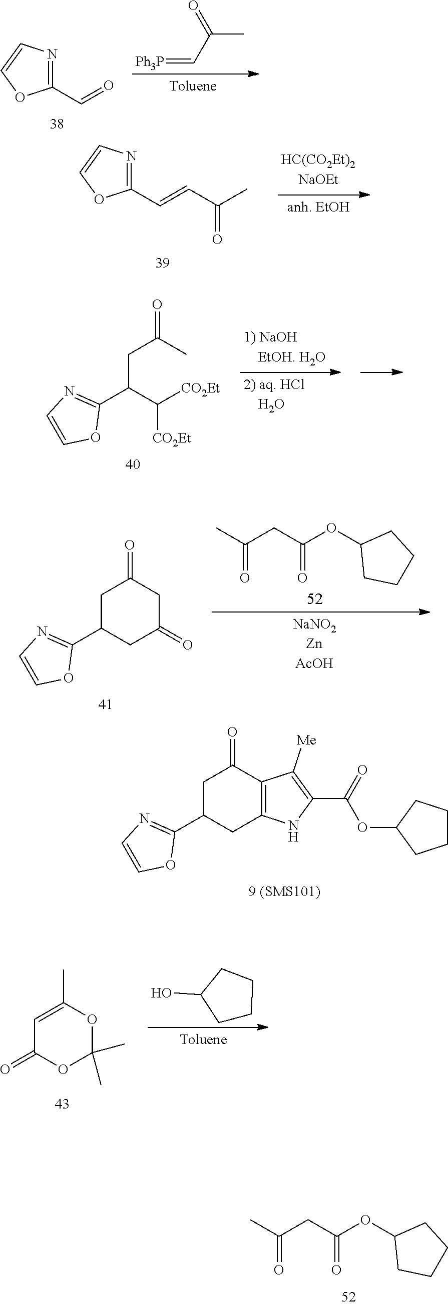

An exemplary synthetic procedure for preparation of pentyl-3-methyl-4-oxo-6-(thiazol-2-yl)-4,5,6,7-tetrahydro-1H-indole-2-carboxylate (SMS213) is as follows:

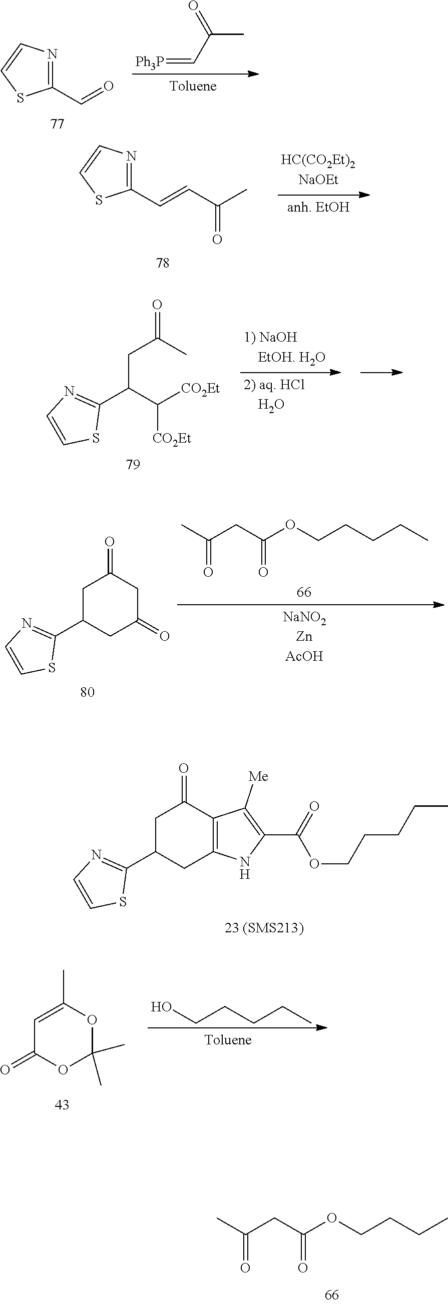

An exemplary synthetic procedure for preparation of pentyl-4-hydroxy-3-methyl-6-(oxazol-2-yl)-4,5,6,7-tetrahydro-1H-indole-2-carboxylate (SMS108) is as follows:

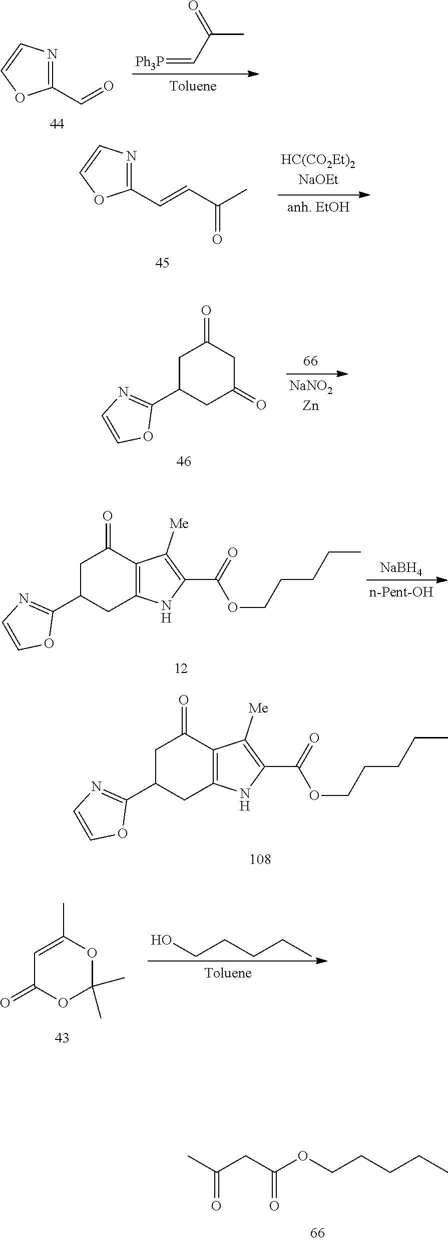

An exemplary synthetic procedure for preparation of propyl-4-hydroxy-3-methyl-6-(oxazol-2-yl)-4,5,6,7-tetrahydro-1H-indole-2-carboxylate (SMS291) is as follows:

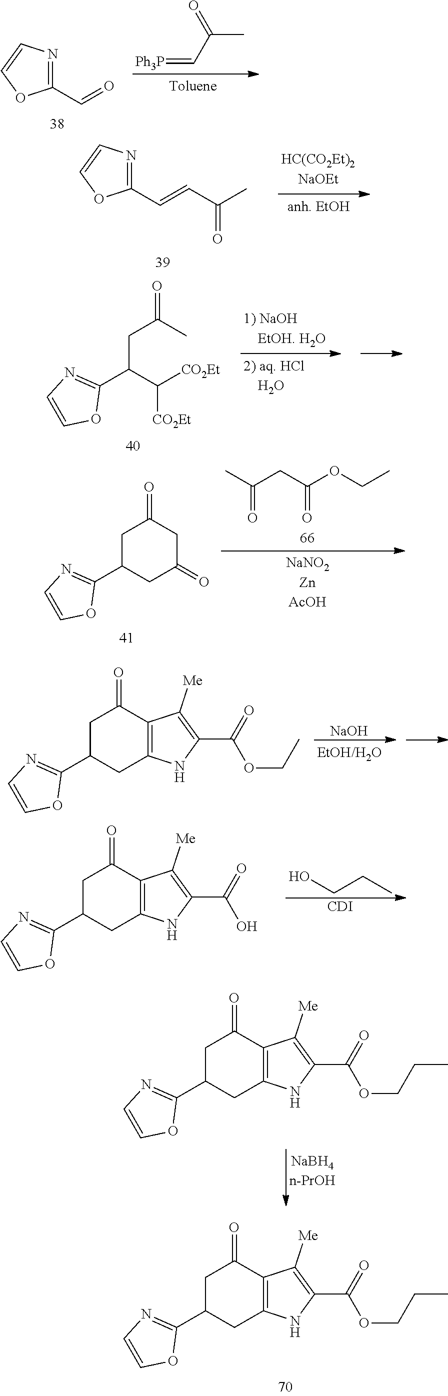

An exemplary synthetic procedure for preparation of 4-ami0butyl-3-methyl-6-(oxazol-2-yl)-4-oxo-4,5,6,7-tetrahydro-1H-indole-2-carboxylate dihydrogen chloride (SMS088) is as follows:

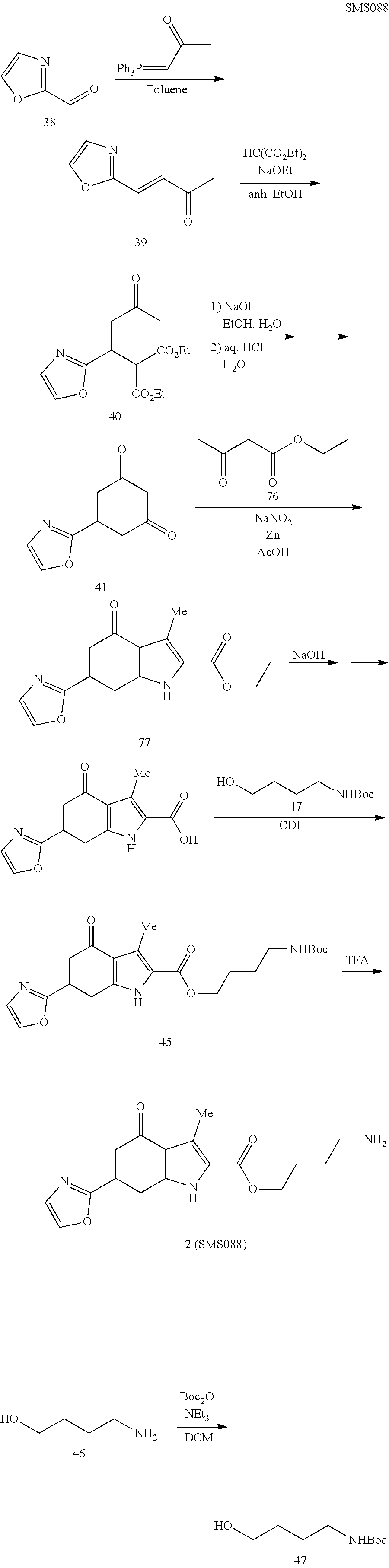

An exemplary synthetic procedure for preparation of 4-hydroxybutyl 3-methyl-6-(oxazol-2-yl)-4-oxo-4,5,6,7-tetrahydro-1H-indole-2-carboxylate (SMS096) is as follows:

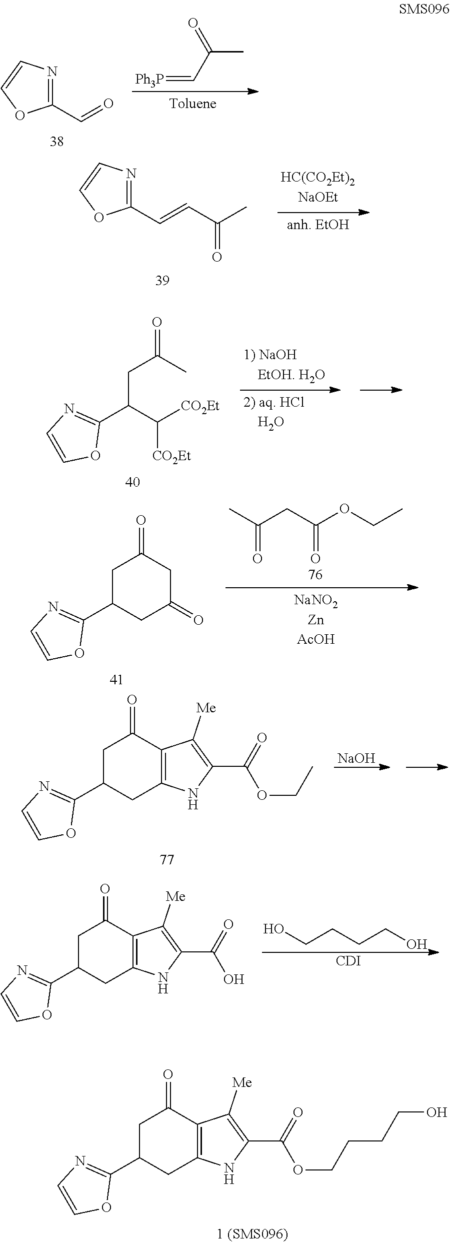

An exemplary synthetic procedure for preparation of 4-((3-methyl-6-(oxazol-2-yl)-4-oxo-4,5,6,7-tetrahydrobenzofuran-2-carbonyl)oxy)butan-1-aminium chloride (SMS228) is as follows:

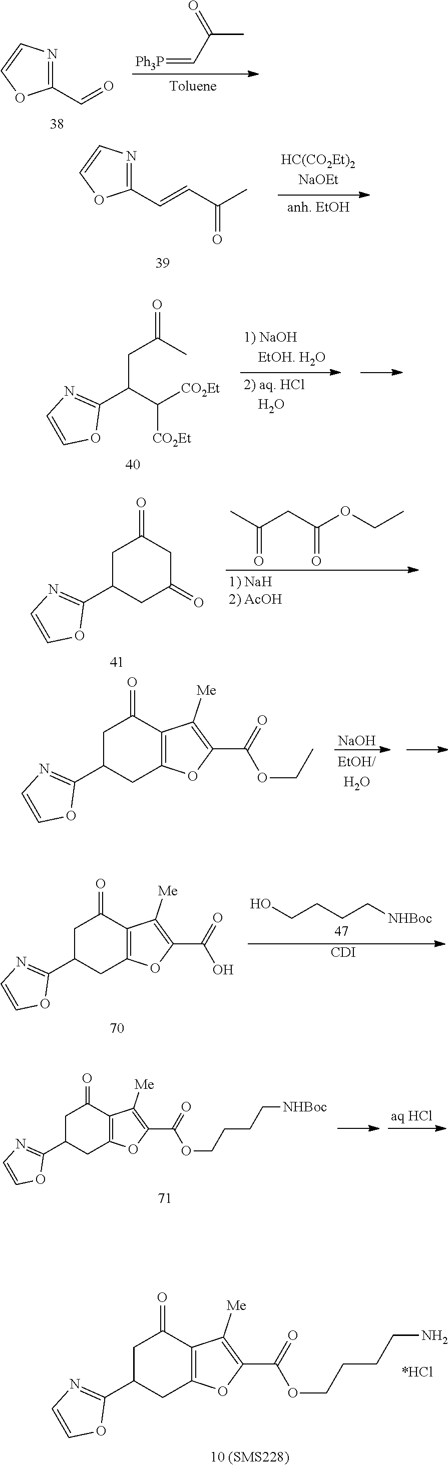

An exemplary synthetic procedure for preparation of 4-hydroxybutyl-3-methyl-6-(oxazol-2-yl)-4-oxo-4,5,6,7-tetrahydrobenzofuran-2-carboxylate (SMS229) is as follows:

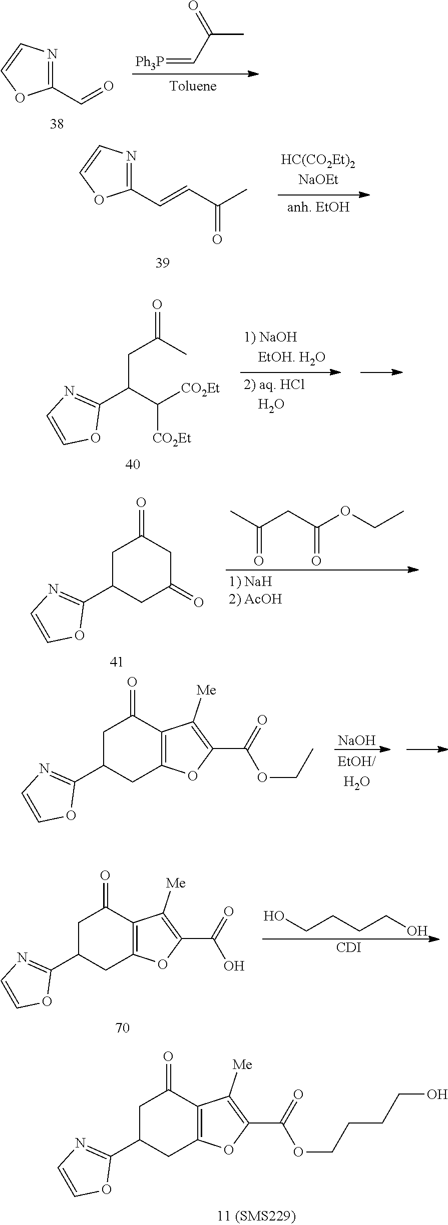

An exemplary synthetic procedure for preparation of Propyl 3-methyl-6-(oxazol-2-yl)-4-oxo-4,5,6,7-tetrahydro-1H-pyrrolo[2,3-c]pyridine-2-carboxylate (CMP 128, SMS294) is as follows:

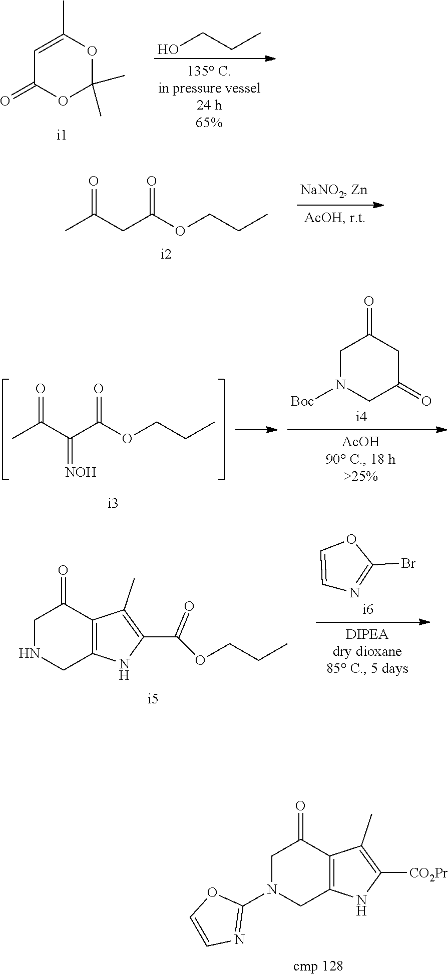

An exemplary synthetic procedure for preparation of Pentyl 3-methyl-4-oxo-6-phenyl-4,5,6,7-tetrahydro-1H-pyrrolo[2,3-c]pyridine-2-carboxylate (CMP 104, SMS078) is as follows:

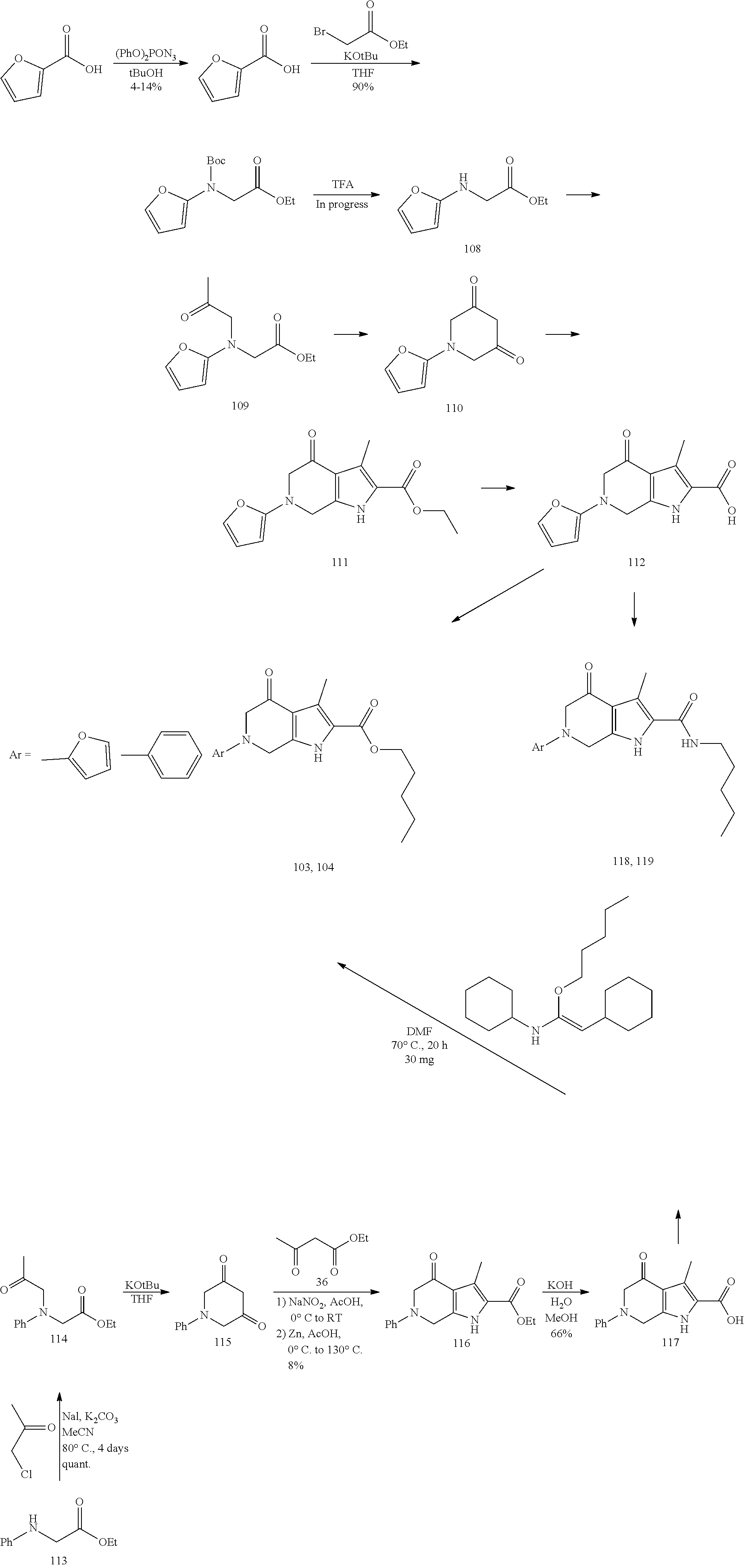

### Example 1

### Solubility

The inventors synthesized a series of oxazole derivatives, schematically presented below (Table 1):

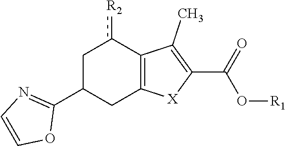

TABLE 1
Oxazole derivatives

| Compound | X | R₁ | R₂ |
|---|---|---|---|
| SMS077 | N | pentyl | O |
| SMS101 | N | cycplopentyl | O |
| SMS088 | N | pentylamine | O |
| SMS096 | N | pentyl alcohol | O |
| SMS108 | N | pentyl | OH |
| SMS291 | N | propyl | OH |
| SMS182 | O | pentyl | O |
| SMS228 | O | pentylamine | O |
| SMS229 | O | pentyl alcohol | O |

Solubility of the synthesized molecules was compared to equivalent molecules having a benzene, furan, thiophene, thiazole or pyridine group instead of oxazole group (Table 2).

TABLE 2
Molecules tested for solubility

| Compound | Structure |
|---|---|
| SMS001 | 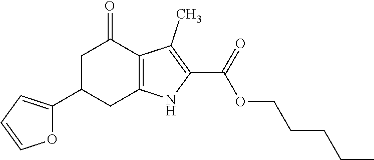 [SMILES: CCCCCOC(=O)c1[nH]c2c(c1C)C(=O)CC(c1ccco1)C2] |
| SMSS077 | 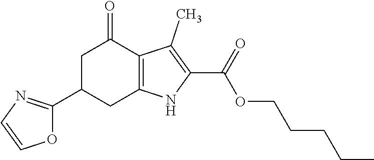 [SMILES: CCCCCOC(=O)c1[nH]c2c(c1C)C(=O)CC(c1ncco1)C2] |
| SMS213 | 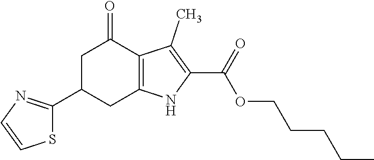 [SMILES: CCCCCOC(=O)c1[nH]c2c(c1C)C(=O)CC(c1nccs1)C2] |
| SMS021 | 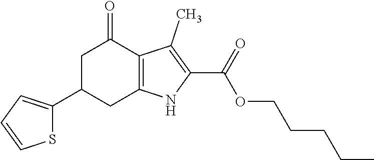 [SMILES: CCCCCOC(=O)c1[nH]c2c(c1C)C(=O)CC(c1cccs1)C2] |
| SMS182 | 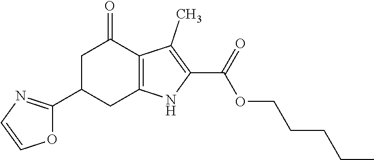 [SMILES: CCCCCOC(=O)c1[nH]c2c(c1C)C(=O)CC(c1ncco1)C2] |
| SMS022 | 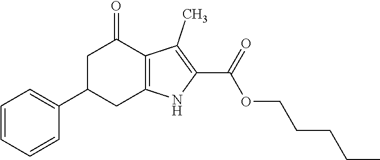 [SMILES: CCCCCOC(=O)c1[nH]c2c(c1C)C(=O)CC(c1ccccc1)C2] |
| SMS074 | 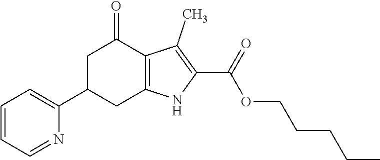 [SMILES: CCCCCOC(=O)c1[nH]c2c(c1C)C(=O)CC(c1ccccn1)C2] |

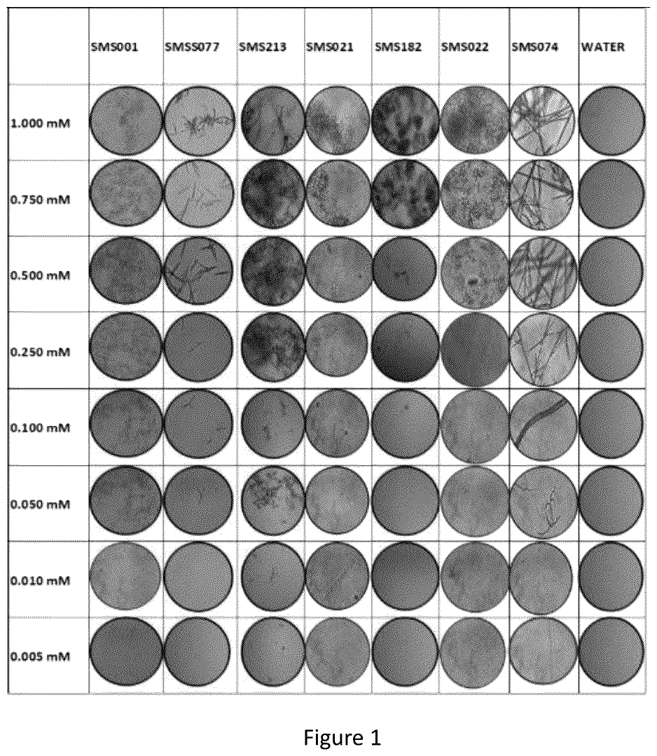

The inventors observed that the oxazole group (SMS077, SMS182) confers better water solubility to the molecule as compared with benzene (SMS022), furan (STK405759 (SMS001)), thiophene (SMS021), thiazole (SMS213) or pyridine groups (SMS074). FIG. 1 shows precipitated crystals and a cloudy appearance at lower concentrations as a consequence of low water solubility.

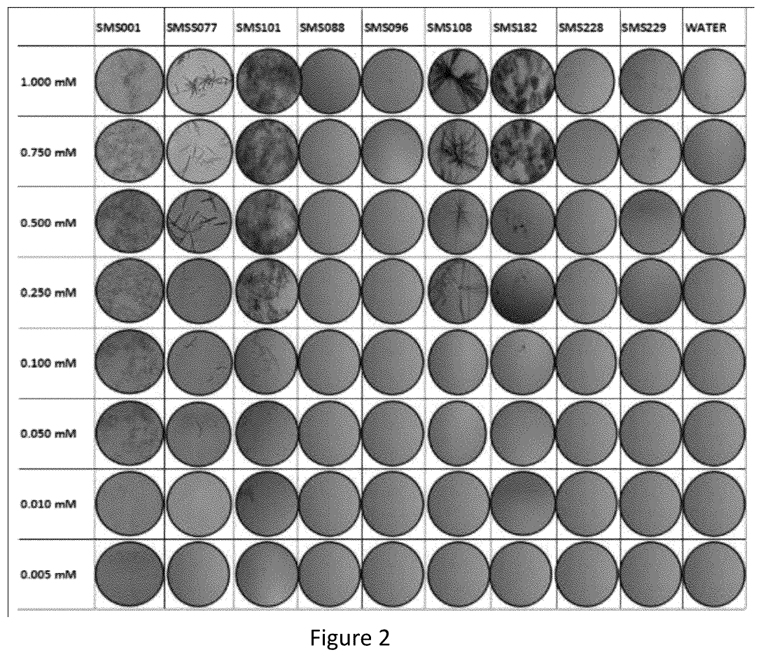

Between the molecules that share an oxazole group, water solubility is higher in molecules with R₁=pentylamine>pentyl alcohol>H>propyl >pentyl>cyclopentyl and with R₂═OH (FIG. 2).

### Example 2

### In Vitro Cytotoxic Activity

The cytotoxic activity of the novel family was evaluated against various tumor cells, including myeloma cells, acute lymphoblastic leukemia cells, acute monocytic leukemia cells, chronic myelogenous leukemia cells, pancreatic cancer cells, breast cancer cells, uterine sarcoma cells, cervix adenocarcinoma cells, ovary cancer cells, melanoma cells, osteosarcoma cells, prostate cancer cells, lung cancer cells, neuroblastoma cells and colorectal adenocarcinoma cells by XTT assay. Compounds SMS077 and SMS182 exhibited very potent cytotoxic activity against different cancer cell lines at low nanomolar levels. SMS077 had an IC₅₀ value that ranged from 10 nM in acute lymphoblastic leukemia cells to 426 nM in metastatic breast adenocarcinoma cells and was lower than that of STK405759 (SMS001) in almost all tested cells.

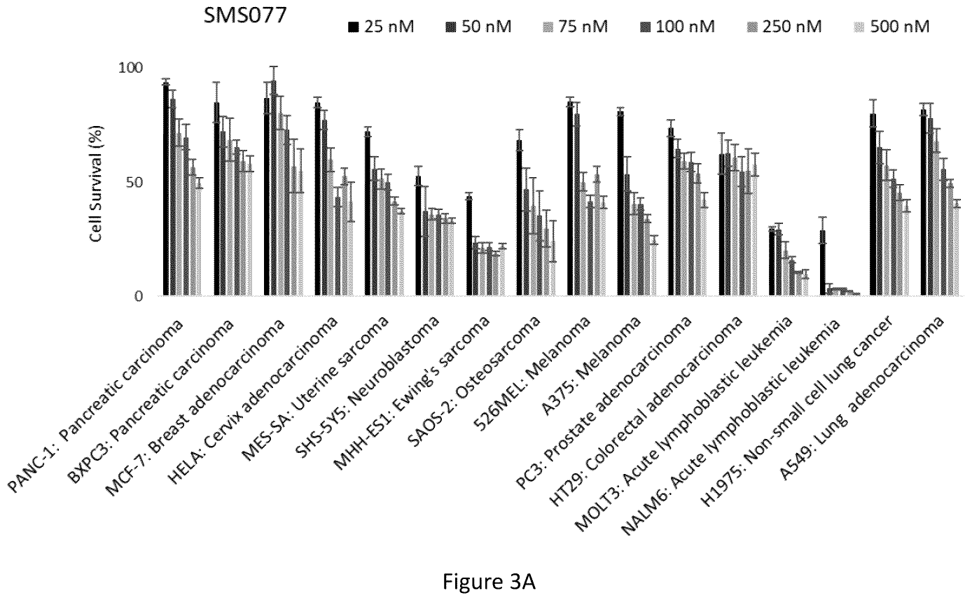

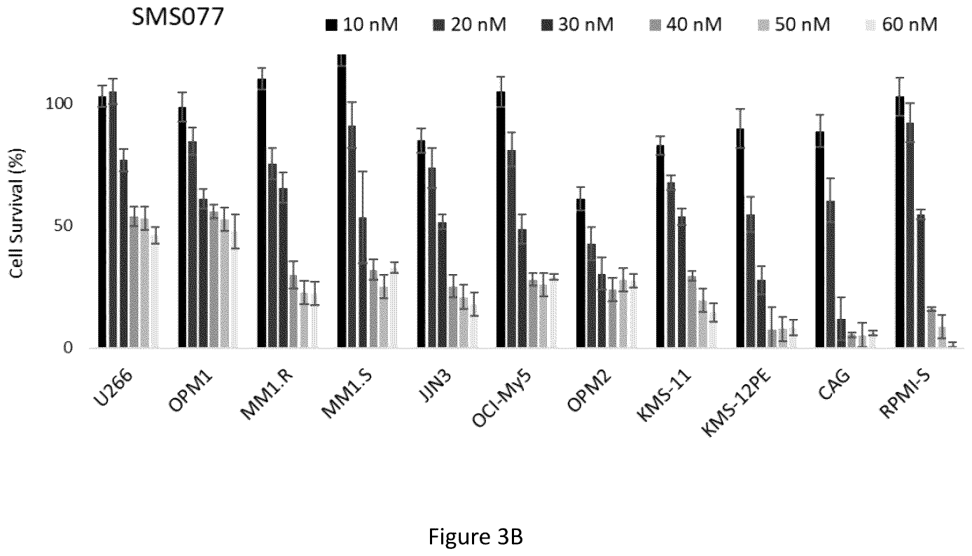

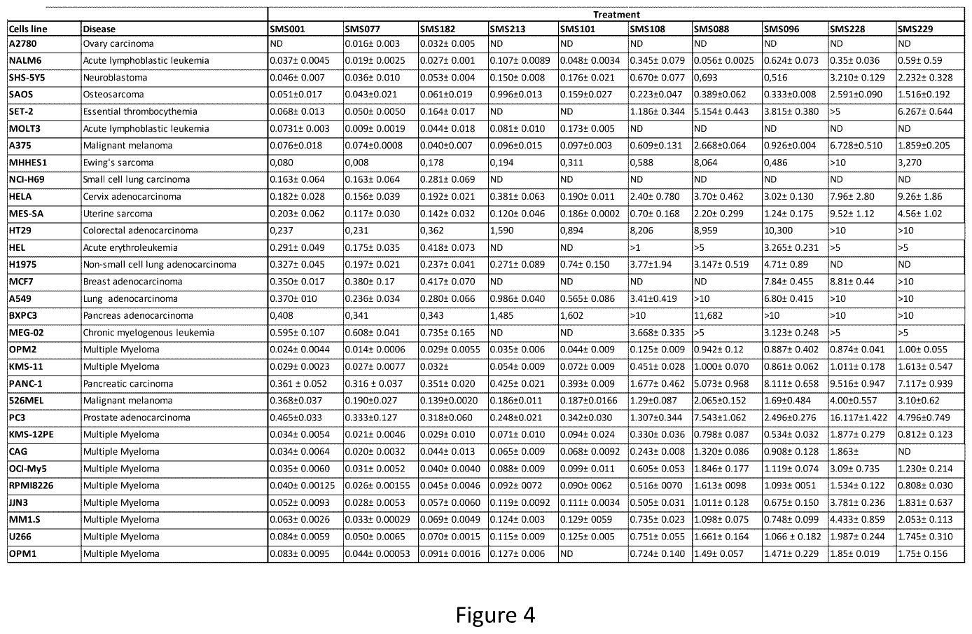

When this family of compounds were tested on different cell lines from the same type of tumor with different phenotypes there were no significant differences in cytotoxic sensitivity. For example, in myeloma cells the IC₅₀ value of SMS077 varied between 14 and 52 nM in 10 different myeloma cell lines tested (FIGS. 3A-B and FIG. 4).

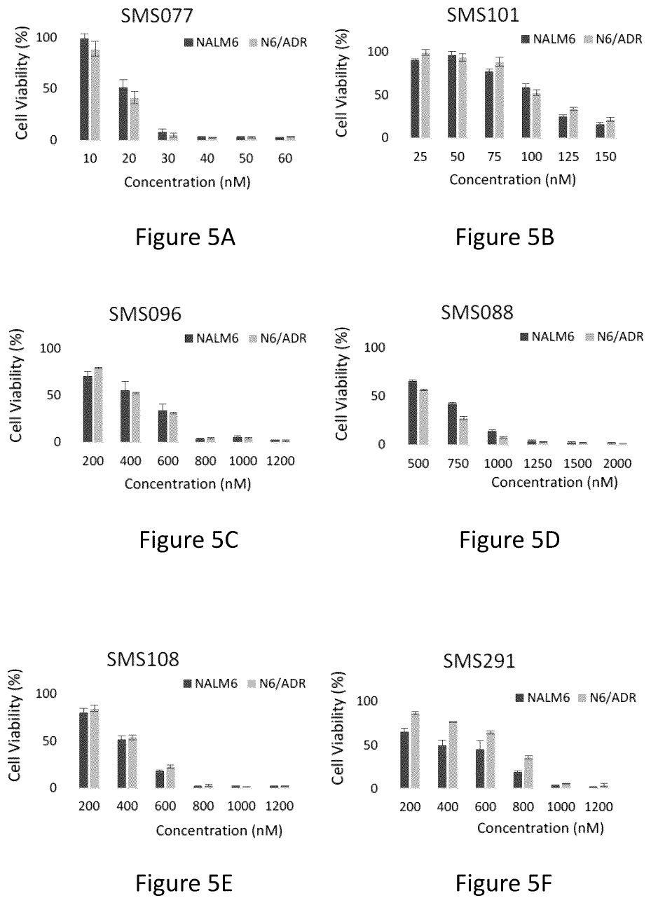

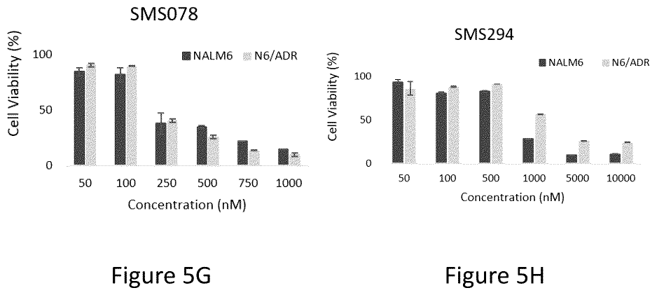

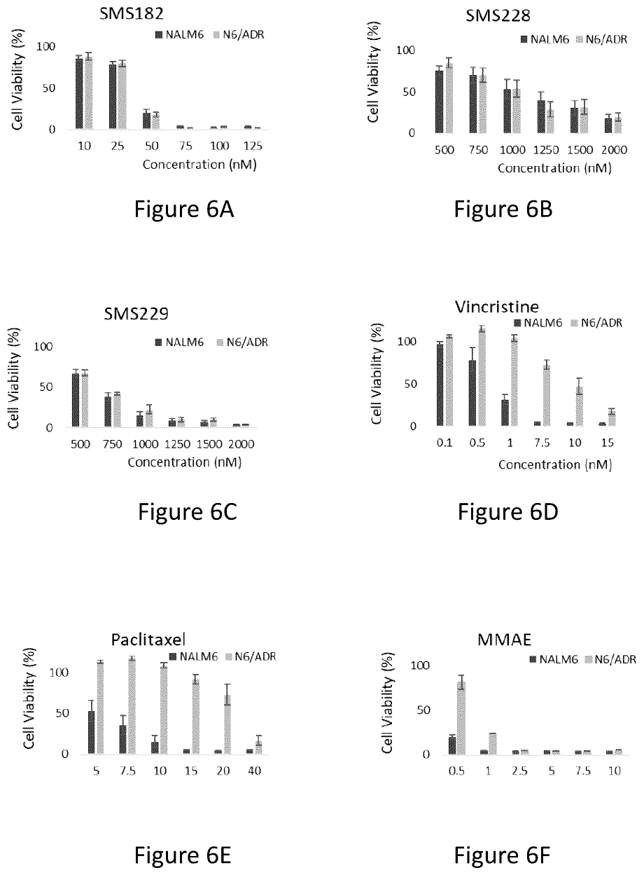

Most tubulin-binding anticancer drugs currently used in the clinic are substrates for Pgp effux pump. The inventors tested the sensitivity of the molecules to this pump by comparing cell viability after treatment between similar paired of cells that their main difference consists in Pgp overexpression. The inventors tested the viability of NALM6 acute lymphoblastic leukemia cell line (ALL) and its corresponding doxorubicin resistant N6/ADR cell line that overexpress Pgp glycoprotein and has cross-resistance to vincristine. Dose response in the two pairs of isogenic cell lines showed no sensitivity of the novel molecules to Pgp glycoprotein overexpression in contrast to VCR, MMAE and taxol treatments which were sensitive to PgP (FIGS. 5A-H and FIGS. 6A-F).

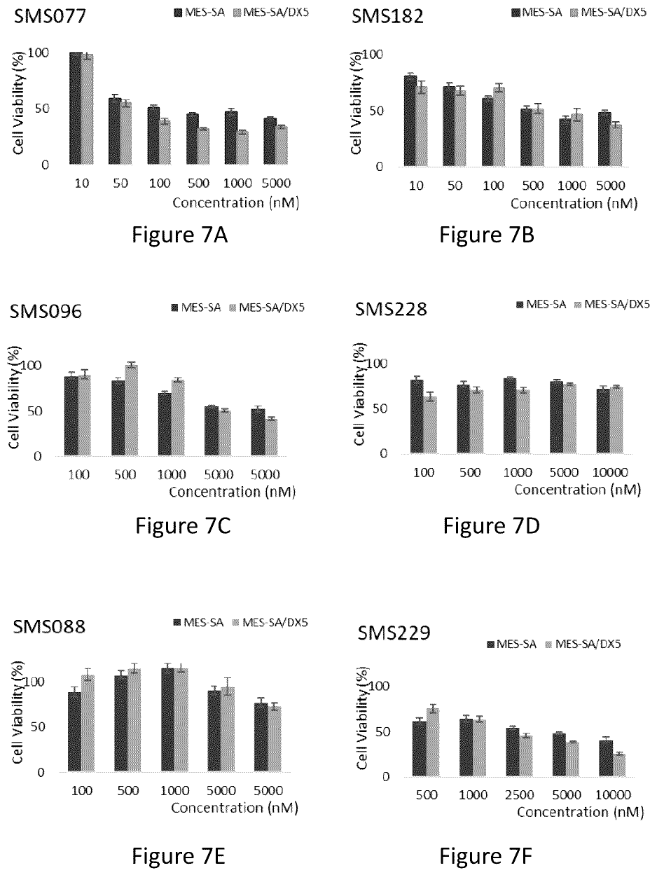

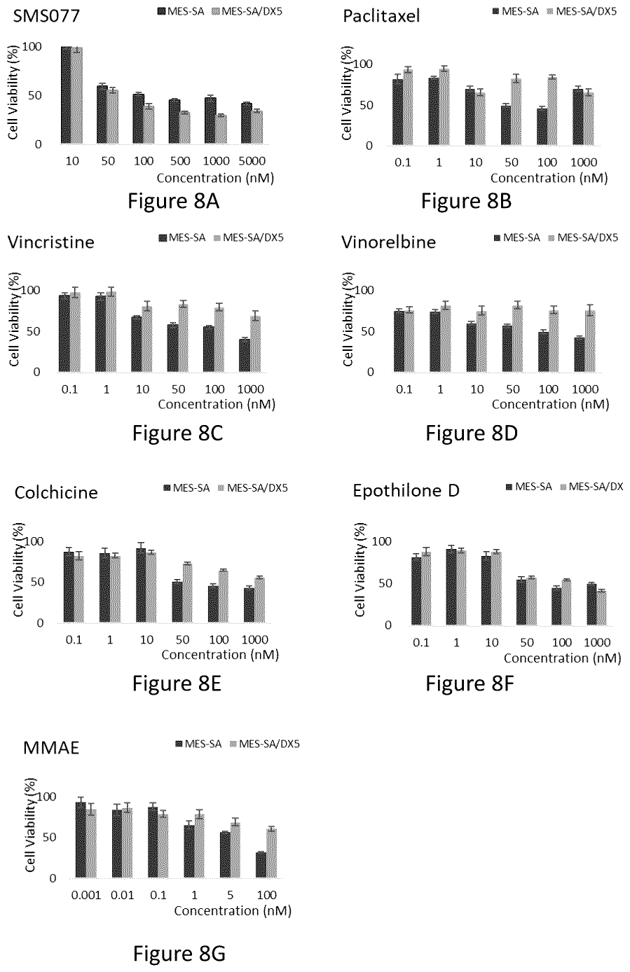

The same effect of cytotoxic activity of the inventors compounds was observed in poorly differentiated uterine sarcoma cells and in their subline that overexpresses glycoprotein P. In contrast, paclitaxel, vincristine, vinorelbine, colchicine and MMAE (FIGS. 7A-F and FIGS. 8A-G) were all less effective in the subline overexpressing PgP.

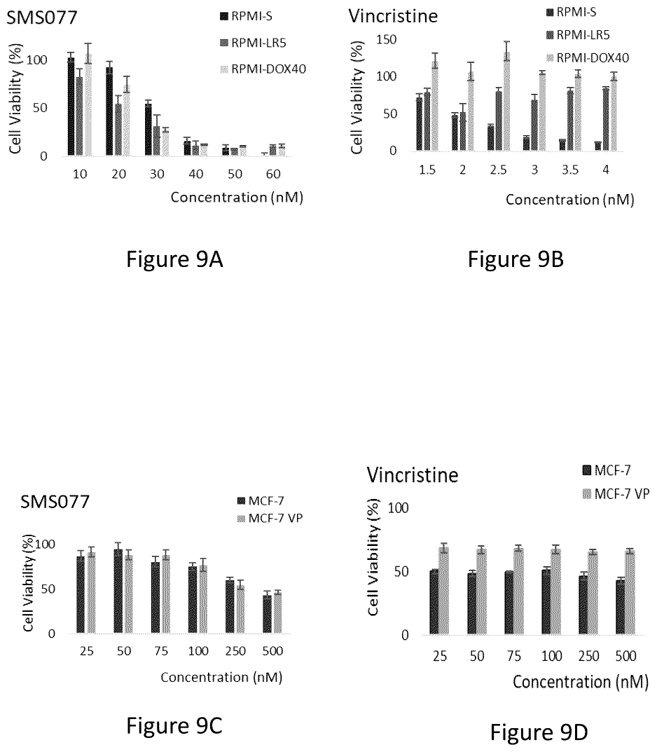

RPMI8226 (RPMI-S) myeloma cells, melphalan (RPMI-LR5) and doxorubicin (RPMI-Dox40) resistant sublines that also have cross-resistance to vincristine, showed no difference in their sensitivity to SMS077 treatment (FIG. 9A-B).

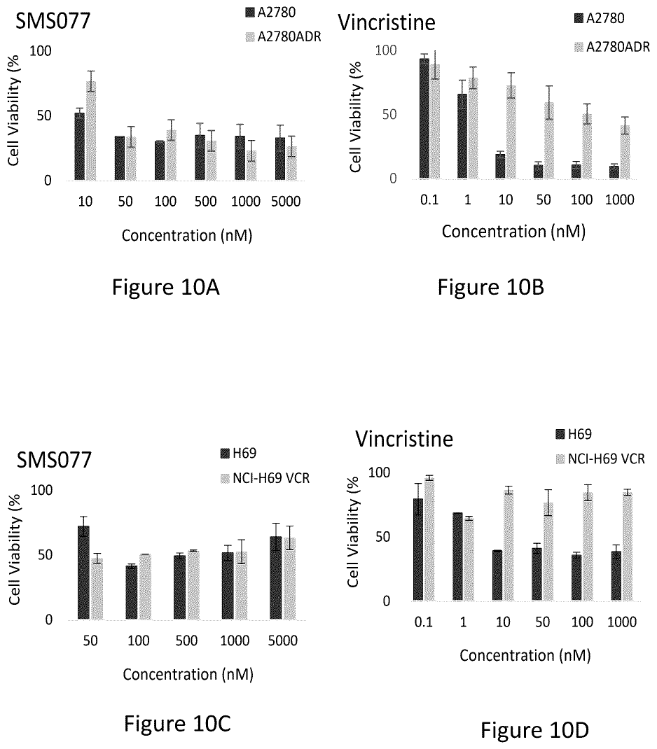

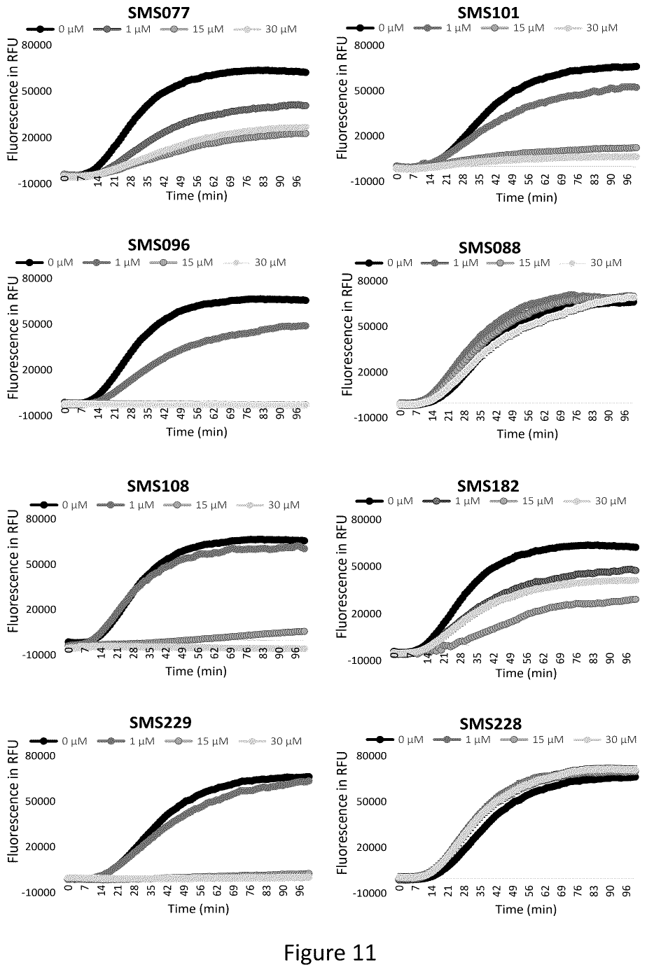

Both the MCF7 metastatic breast adenocarcinoma cells and their corresponding etoposide resistant subline that has cross-resistance to vincristine MCF7-VP and overexpress Pgp glycoprotein showed same sensitivity to SMS077 treatment (FIG. 9C-D). Furthermore, A2780ADR ovary carcinoma cells and NCI-N69-VCR lung carcinoma cells are resistant to vincristine but not to SMS077 treatment (FIGS. 10A-D).

Tables 3-6 present the results of IC₅₀ (half maximal inhibitory concentration) ratio between resistant and non-resistant cells of the compounds SMS001, SMS077, SMS182, SMS213, SMS101, SMS108, SMS088, SMS228 (Table 4) and compounds SMS22, SMS096, SMS291, SMS078 and SMS294 (Table 5) in different types of cancer cell lines (Table 3), in comparison with microtubule targeting agents in clinical use currently (Table 6); ND: non determined.

TABLE 3
List of different resistant and non-resistant
types of cancer cell lines tested

| | | | Mechanism of |
|---|---|---|---|
| Cells line | Disease | Resistance drugs | resistance |
| RPMI 8226 | Multiple Myeloma | | |
| RPMI- | Multiple Myeloma | Doxorubicin, | P-gp |
| DOX40 | | Vincristine, Taxol, | overexpression |
| RPMI 8226 | Multiple Myeloma | | |
| RPMI-LR5 | Multiple Myeloma | Melphalan, | |
| | | Vincristine, Taxol | |
| RPMI 8226 | Multiple Myeloma | | |
| RPMI- | | Mitoxantrone | |
| MR20 | | | |
| MM1.S | Multiple Myeloma | | |
| MM1.R | Multiple Myeloma | Dexamethasone | |
| NALM6 | Acute lymphoblastic | | |
| | leukemia | | |
| N6/ADR | Acute lymphoblastic | Etoposide, | P-gp |
| | leukemia | Vincristine, | overexpression |
| | | Actinomycin D | |
| MCF7 | Breast | | |
| | adenocarcinoma | | |
| MCF7-VP | Breast | Epipodophyllotoxins | MRP |
| | adenocarcinoma | VP-16 and VM-26 | overexpression |
| MES-SA | Poorly differentiated | | |
| | uterine sarcoma | | |
| MES- | Poorly differentiated | Doxorubicin, | P-gp |
| SA/DX5 | uterine sarcoma | Daunorubicin, | overexpression |
| | | Dactinomycin, | |
| | | Vincristine, Taxol, | |
| | | Colchicine, | |
| | | Melphalan | |
| NCI-H69 | Small cell lung | | |
| | carcinoma | | |
| NCI-H69 | Small cell lung | Vincristine | |
| VCR/R | carcinoma | | |
| A2780 | Ovarian carcinoma | | |
| A2780ADR | Ovarian carcinoma | Adriamycin, | |
| | | Melphalan and | |
| | | Vinblastine | |

TABLE 4
IC₅₀ (half maximal inhibitory concentration) ratio between resistant and non-resistant
cells of the indicated molecules in different types of cancer cell lines

| Cells line | Parameter | SMS001 | SMS077 | SMS182 | SMS213 | SMS101 | SMS108 | SMS088 | SMS228 |
|---|---|---|---|---|---|---|---|---|---|
| RPMI | IC50 | 0.040 ± | 0.026 ± | 0.045 ± | 0.092 ± | 0.090 ± | 0.516 ± | 1.613 ± | 1.534 ± |
| 8226 | | 0.00125 | 0.00155 | 0.0046 | 0072 | 0062 | 0070 | 0098 | 0.122 |
| RPMI- | IC50 | 0.034 ± | 0.025 ± | 0.037 ± | 0.068 ± | 0.077 ± | 0.498 ± | 1.353 ± | 1.309 ± |
| DOX40 | | 0.0040 | 0.0030 | 0.0032 | 0.006 | 0011 | 0.029 | 0.051 | 0.045 |
| | RATIO | 0.850 | 0.962 | 0.822 | 0.739 | 0.856 | 0.965 | 0.839 | 0.853 |
| RPMI | IC50 | 0.040 ± | 0.026 ± | 0.045 ± | 0.092 ± | 0.090 ± | 0.516 ± | 1.613 ± | 1.534 ± |
| 8226 | | 0.00125 | 0.00155 | 0.0046 | 0072 | 0062 | 0070 | 0098 | 0.122 |
| RPMI- | IC50 | 0.029 ± | 0.019 ± | 0.034 ± | 0.060 ± | 0.073 ± | 0.427 ± | 1.427 ± | 1.299 ± |
| LR5 | | 0.0027 | 0.0025 | 0.0019 | 0.0010 | 00034 | 0.005 | 0.134 | 0.077 |
| | RATIO | 0.725 | 0.731 | 0.756 | 0.652 | 0.811 | 0.828 | 0.885 | 0.847 |
| RPMI | IC50 | 0.040 ± | 0.026 ± | 0.045 ± | 0.092 ± | 0.090 ± | 0.516 ± | 1.613 ± | 1.534 ± |
| 8226 | | 0.00125 | 0.00155 | 0.0046 | 0072 | 0062 | 0070 | 0098 | 0.122 |
| RPMI- | IC50 | 0.035 ± | 0.023 ± | 0.036 ± | 0.079 ± | 0.099 ± | 0.388 ± | >1.8 | 2.230 ± |
| MR20 | | 0.0043 | 0.0020 | 0.0086 | 0.003 | 0058 | 0.049 | | 0.465 |
| | RATIO | 0.875 | 0.885 | 0.800 | 0.859 | 1.100 | 0.752 | >1.116 | >1.454 |
| MM1.S | IC50 | 0.063 ± | 0.033 ± | 0.069 ± | 0.124 ± | 0.129 ± | 0.735 ± | 1.098 ± | 4.433 ± |
| | | 0.0026 | 0.00029 | 0.0049 | 0.003 | 0059 | 0.023 | 0.075 | 0.859 |
| MM1.R | IC50 | 0.069 ± | 0.036 ± | 0.095 ± | 0.147 ± | 0.510 | 1.20 ± | 1.025 ± | >1.8 |
| | | 0.007 | 0.0057 | 0.010 | 0.002 | | 0.048 | 0.299 | |
| | RATIO | 1.095 | 1.091 | 1.377 | 1.185 | 3.953 | 1.633 | 0.934 | >0.406 |
| NALM6 | IC50 | 0.037 ± | 0.019 ± | 0.027 ± | 0.107 ± | 0.048 ± | 0.345 ± | 0.056 ± | 0.35 ± |
| | | 0.0045 | 0.0025 | 0.001 | 0.0089 | 0.0034 | 0.079 | 0.0025 | 0.036 |
| N6/ADR | IC50 | 0.037 ± | 0.017 ± | 0.027 ± | 0.109 ± | 0.042 ± | 0.33 ± | 0.062 ± | 0.36 ± |
| | | 0.0005 | 0.002 | 0.002 | 0.006 | 0.004 | 0.03 | 0.0033 | 0.017 |
| | RATIO | 1.000 | 0.895 | 1.000 | 1.019 | 0.875 | 0.957 | 1.107 | 1.029 |
| MCF7 | IC50 | 0.350 ± | 0.380 ± | 0.417 ± | 0.243 | 11.948 | 7.125 | 5.836 | 8.81 ± |
| | | 0.017 | 0.17 | 0.070 | | | | | 0.44 |
| MCF7- | IC50 | 0.469 ± | 0.560 ± | 0.440 ± | 0.513 ± | 0.351 ± | 5.90 ± | 7.30 ± | 12.33 ± |
| VP | | 0.046 | 0.077 | 0.047 | 0.09 | 0.07 | 0.86 | 1.13 | 1.15 |
| | RATIO | 1.340 | 1.474 | 1.055 | 2.114 | 0.029 | 0.828 | 1.251 | 1.400 |
| MES-SA | IC50 | 0.203 ± | 0.128 ± | 0.244 ± | 0.120 ± | 0.186 ± | 0.70 ± | 2.20 ± | 9.52 ± |
| | | 0.062 | 0.041 | 0.078 | 0.046 | 0.0002 | 0.168 | 0.299 | 1.12 |
| MES- | IC50 | 0.049 ± | 0.065 ± | 0.130 ± | 0.139 ± | 0.213 ± | 0.49 ± | 5.26 ± | 7.41 ± |
| SA/DX5 | | 0.005 | 0.0031 | 0.041 | 0.0023 | 0.007 | 0.05 | 0.73 | 1.02 |
| | RATIO | 0.241 | 0.508 | 0.533 | 1.158 | 1.145 | 0.700 | 2.391 | 0.778 |
| NCI-H69 | IC50 | ND | 0.163 ± | 0.281 ± | ND | ND | ND | ND | ND |
| | | | 0.064 | 0.069 | | | | | |
| NCI-H69 | IC50 | ND | 0.048 ± | 0.073 ± | ND | ND | ND | ND | ND |
| VCR/R | | | | | | | | | |
| | RATIO | | 0.294 | 0.260 | | | | | |
| A2780 | IC50 | ND | 0.016 ± | 0.032 ± | ND | ND | ND | ND | ND |
| | | | 0.003 | 0.005 | | | | | |
| A2780ADR | IC50 | ND | 0.031 ± | 0.035 ± | ND | ND | ND | ND | ND |
| | | | 0.0056 | 0.0084 | | | | | |
| | RATIO | | 1.938 | 1.094 | | | | | |

TABLE 5
IC₅₀ ratio between resistant and non-resistant cells of the
indicated molecules, in different types of cancer cell lines

| Cells line | Parameter | SMS229 | SMS096 | SMS291 | SMS078 | SMS294 |
|---|---|---|---|---|---|---|
| RPMI | IC50 | 0.808 ± | 1.093 ± | 0.978 | 0.210 ± | 0.666 ± |
| 8226 | | 0.030 | 0051 | | 0.013 | 0.090 |
| RPMI- | IC50 | 0.933 ± | 1.1813 ± | 0.531 | 0.489 ± | 1.064 ± |
| DOX40 | | 0.039 | 0.108 | | 0.0221 | 0.337 |
| | RATIO | 1.155 | 1.081 | 0.543 | 2.329 | 1.598 |
| RPMI | IC50 | 0.808 ± | 1.093 ± | ND | ND | ND |
| 8226 | | 0.030 | 0051 | | | |
| RPMI- | IC50 | 0.958 ± | 1.127 ± | ND | ND | ND |
| LR5 | | 0.050 | 0.165 | | | |
| | RATIO | 1.186 | 1.031 | | | |
| RPMI | IC50 | 0.808 ± | 1.093 ± | ND | ND | ND |
| 8226 | | 0.030 | 0051 | | | |
| RPMI- | IC50 | 1.548 ± | >1.8 | ND | ND | ND |
| MR20 | | 0.029 | | | | |
| | RATIO | 1.916 | >1.647 | | | |
| MM1.S | IC50 | 2.053 ± | 0.748 ± | ND | ND | ND |
| | | 0.113 | 0.099 | | | |
| MM1.R | IC50 | >1.6 | 1.082 ± | ND | ND | ND |
| | | | 0.050 | | | |
| | RATIO | >0.779 | 1.447 | | | |
| NALM6 | IC50 | 0.59 ± | 0.624 ± | 0.51 ± | 0.178 ± | 0.907 |
| | | 0.59 | 0.073 | 0.05 | 0.02 | |
| N6/ADR | IC50 | 0.67 ± | 0.054 ± | 0.35 ± | 0.165 ± | 1.939 |
| | | 0.099 | 0.012 | 0.081 | 0.017 | |
| | RATIO | 1.136 | 0.087 | 0.686 | 0.927 | 2.138 |
| MCF7 | IC50 | 33.28 ± | 7.84 ± | ND | ND | ND |
| | | 2.93 | 0.455 | | | |
| MCF7- | IC50 | 11.98 ± | 5.74 ± | ND | ND | ND |
| VP | | 1.47 | 0.61 | | | |
| | RATIO | 0.360 | 0.732 | | | |
| MES-SA | IC50 | 4.56 ± | 1.24 ± | ND | ND | ND |
| | | 1.02 | 0.175 | | | |
| MES- | IC50 | 3.19 ± | 1.43 ± | ND | ND | ND |
| SA/DX5 | | 0.04 | 0.11 | | | |
| | RATIO | 0.700 | 1.153 | | | |

TABLE 6
IC₅₀ ratio between resistant and non-resistant cells of the commercially used
microtubule targeting agents, in different types of cancer cell lines

| Cells line | Paqlitaxel | Colchicine | Vincristine | MMAE | Vinorelbine | Epothilone |
|---|---|---|---|---|---|---|
| RPMI | ND | 0.010 ± | 0.0019 ± | <0.0025 | ND | |
| 8226 | | 0.0014 | 0.00021 | | | |
| RPMI- | ND | >0.06 | >0.010 | >0.015 | ND | |
| DOX40 | | | | | | |
| | | >6.000 | >5.263 | >6.000 | | |
| RPMI | ND | 0.010 ± | 0.0019 ± | <0.0025 | ND | |
| 8226 | | 0.0014 | 0.00021 | | | |
| RPMI-LR5 | ND | >0.06 | >0.004 | >0.015 | ND | |
| | | >6.000 | >2.105 | >6.000 | | |
| RPMI | ND | 0.010 ± | 0.0019 ± | ND | ND | |
| 8226 | | 0.0014 | 0.00021 | | | |
| RPMI- | ND | 0.033 ± | 0.0003 ± | ND | ND | |
| MR20 | | 0.016 | 0.0001 | | | |
| | | 3.300 | 0.158 | | | |
| MM1.S | ND | 0.013 ± | 0.0016 ± | <0.0025 | ND | |
| | | 0.0015 | 0.0001 | | | |
| MM1.R | ND | 0.27 ± | 0.0017 ± | <0.0025 | ND | |
| | | 0.007 | 0.0002 | | | |
| | | 20.769 | 1.063 | | | |
| NALM6 | 0.0084 ± | ND | 0.0011 ± | 0.0003 ± | ND | |
| | 0.0015 | | 0.00006 | 0.00005 | | |
| N6/ADR | 0.024 ± | ND | 0.012 ± | 0.0008 ± | ND | |
| | 0.0046 | | 0.0016 | 0.00003 | | |
| | 2.857 | ND | 10.909 | 2.667 | | |
| MCF7 | 0.136 ± | 0.805 ± | 0.078 ± | ND | 0.022 ± | 0.22 ± |
| | 0.047 | 0.013 | 0.022 | | 0.0015 | 0.0035 |
| MCF7-VP | 0.074 ± | 0.740 ± | 0.220 ± | ND | 0.028 ± | 0.456 ± |
| | 0.0003 | 0.190 | 0.019 | | 0.0004 | 0.044 |
| | 0.544 | 0.919 | 2.821 | | 1.273 | 2.073 |
| MES-SA | 0.110 ± | 0.099 ± | 0.999 ± | 0.016 ± | 0.064 ± | 0.052 ± |
| | 0.028 | 0.017 | 0.021 | 0.0021 | 0.003 | 0.0092 |
| MES- | >1 | 0.70 ± | 3.68 ± | 2.736 ± | >1.00 | 0.187 ± |
| SA/DX5 | | 0.167 | 0.92 | 0.92 | | 0.024 |
| | >9.009 | 7.071 | 3.684 | 165.037 | >15.625 | 3.613 |
| NCI-H69 | 0.059 ± | ND | 0.024 ± | 0.003 ± | 0.022 ± | 0.088 ± |
| | 0.0039 | | 0.008 | 0.0066 | 0.01 | 0.0061 |
| NCI-H69 | >1 | ND | >1 | 0.002 ± | >1 | >1 |
| VCR/R | | | | 0.0021 | | |
| | >16.949 | | >41.667 | 0.667 | >45.454 | >11.364 |
| A2780 | 0.0065 ± | ND | 0.003 ± | 0.0008 ± | 0.009 ± | 0.0027 ± |
| | 0.0014 | | 0.0013 | 0.00026 | 0.0013 | 0.0005 |
| A2780ADR | 0.40 ± | ND | 0.231 ± | 0.025 ± | 2.70 ± | >1 |
| | 0.16 | | 0.029 | 0.004 | 0.055 | |
| | 61.538 | | 77.000 | 31.250 | 300.000 | >370.370 |

### Example 3

### Inhibition of Tubulin Polymerization Activity

The effect of the compounds on their ability to inhibit tubulin polymerization, was tested against a vehicle control by evaluating polymerization of purified tubulin in a cell-free assay where tubulin (2.00 mg/mL) was exposed to vehicle control or 1, 15 and 30 μM of the compounds (n=2). Polymerizations were tracked by an increase in fluorescence excitation wavelength is 340-360 nm and emission wavelength at 410-460 nm. Fluorescence was monitored at 37° C. every 30 sec for 100 min.

SMS077, SMS182, SMS101, SMS108, SMS096 and SMS229 inhibited the rate of tubulin polymerization in a concentration-dependent manner as compared with the vehicle that showed an initial and rapid increase in polymerization. The effect was higher for SMS077 and SMS182 as compared with the other molecules. The presence of a pentylamine in R₁ prevented tubulin polymerization inhibition in a cell free system (FIG. 11).

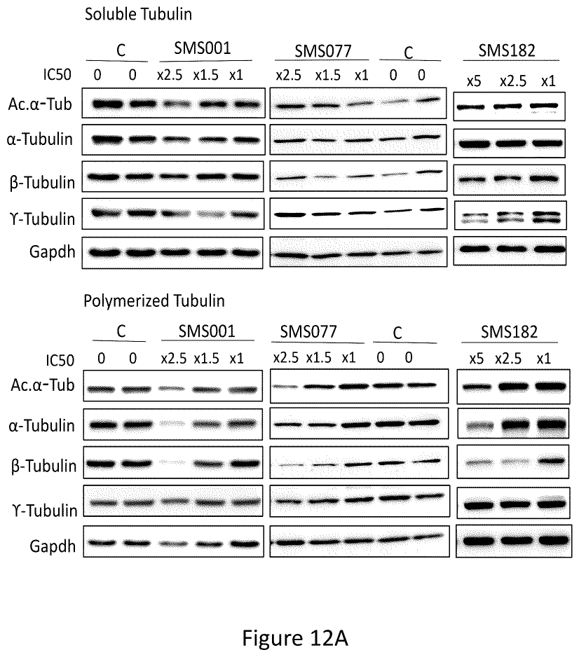

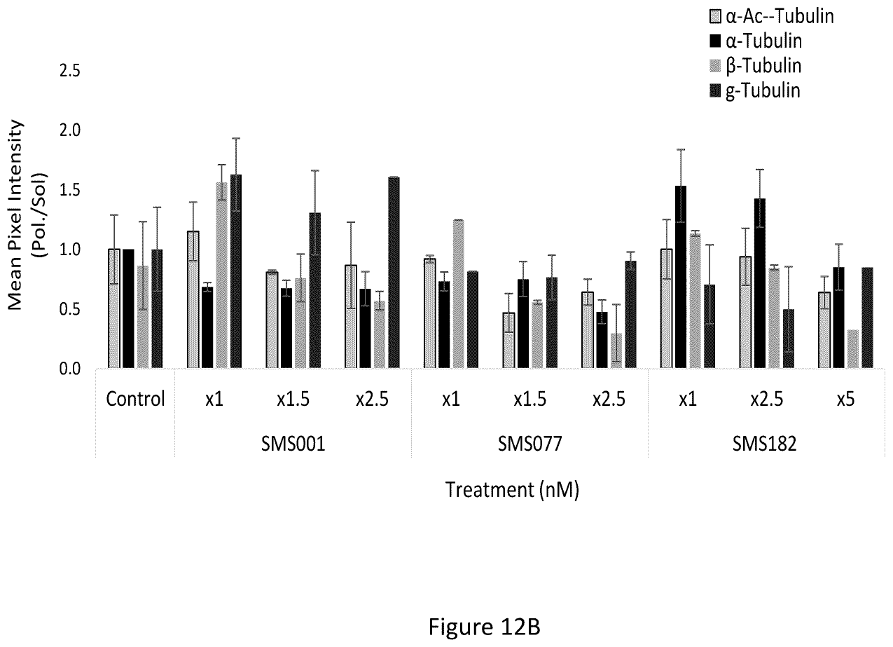

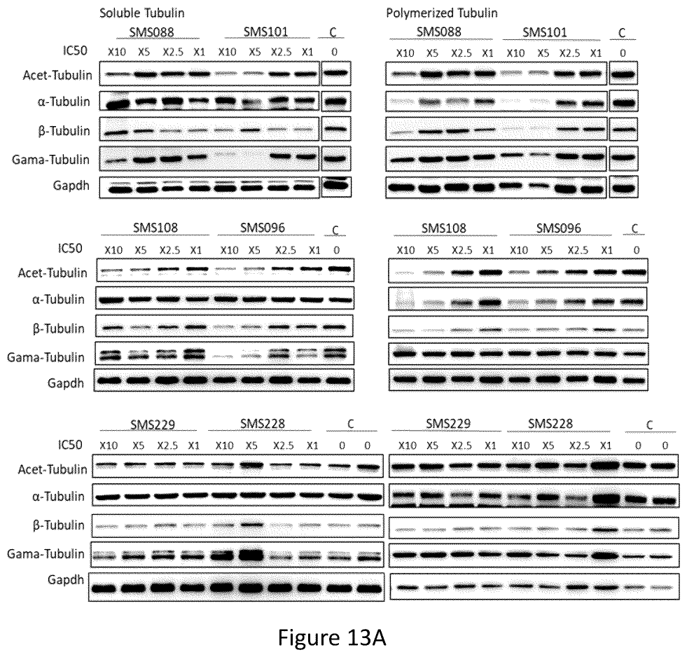

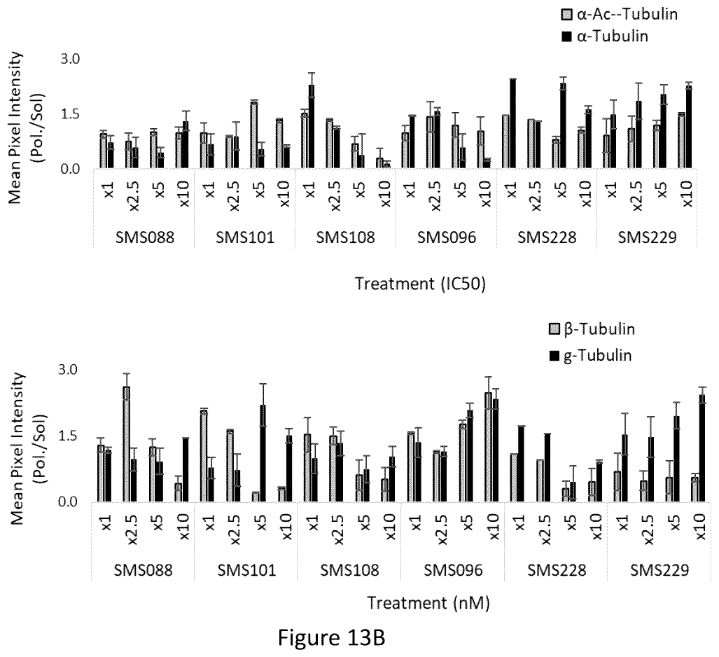

To further examine whether these molecules affect tubulin polymerization in cultured cells, RPMI8226 cells were exposed to the molecules, followed by the assessment of polymerized and unpolymerized tubulin. The level of soluble versus polymerized forms of tubulin was analyzed by western blot analysis in lysates from cultured RPMI8226 myeloma cells treated with the compounds for 18 hours. SMS077, SMS182, SMS088, SMS101, SMS108, SMS096, SMS228 and SMS229 reduced the amount of polymerized a and R tubulin relative to the soluble form in a dose dependent manner (FIGS. 12A-B and FIGS. 13A-B). These results indicated that the molecules destabilized microtubules.

### Example 4

Interaction with Colchicine Binding Site on Microtubules

Microtubule-targeting agents interact with tubulin at four major binding sites: the taxane and laulimalide/peloruside A sites for microtubule-stabilizing agents, and the vinca and colchicine sites for microtubule-destabilizing agents.

To examine whether these molecules bind to the colchicine binding site in cultured cells, an EBI competition assay was performed. EBI could produce a β-tubulin adduct which could be detected by Western blot as a second immuno-reactive β-tubulin band that migrates faster than β-tubulin itself, while if EBI is added to cells previously treated with a colchicine-site binder, the EBI adduct cannot be observed.

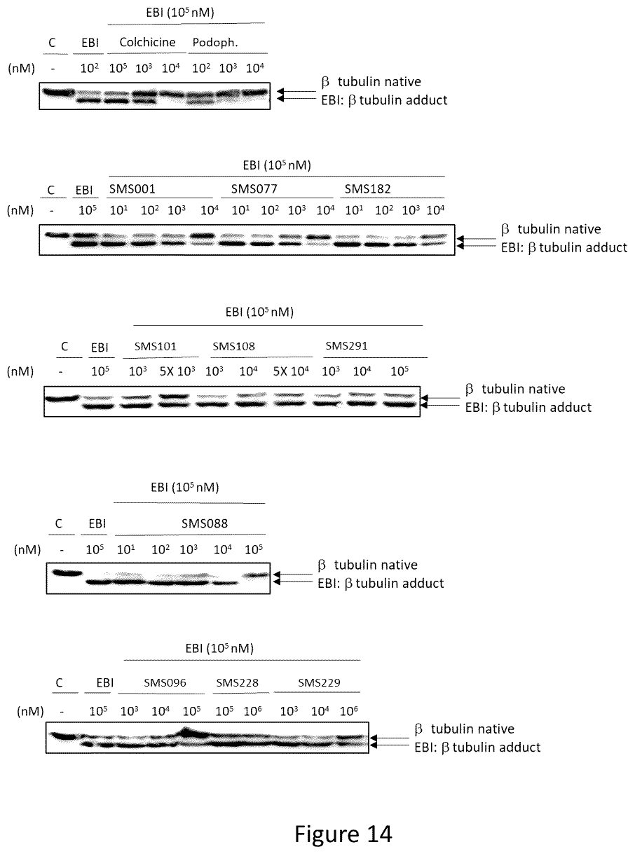

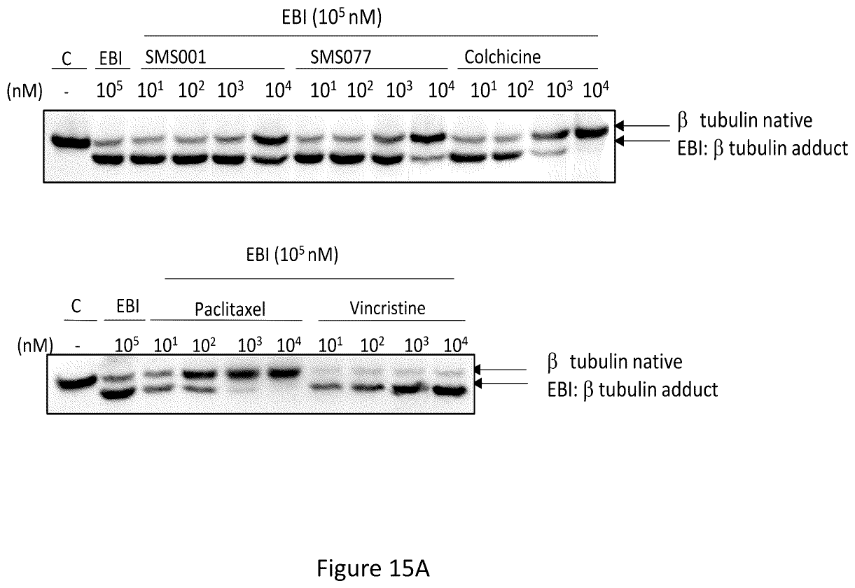

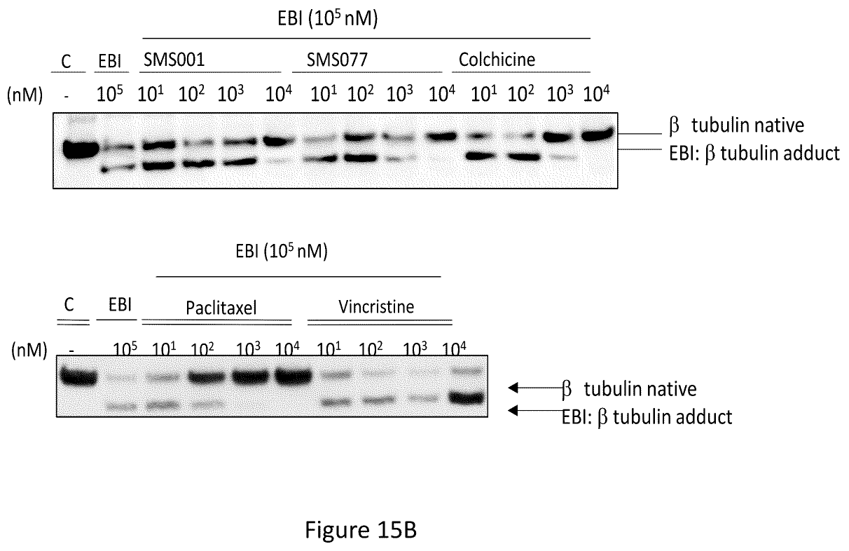

Incubation of SMS001, SMS077, SMS182, SMS101, SMS088, SMS096 and SMS229 molecules, prevented the covalent binding of EBI to the colchicine binding site on tubulin protein in RPMI-8226 living cells resulting in the disappearance of the second immunoreacting band of β-tubulin on SDS-PAGE. However, SMS228, SMS108 and SMS291 did not prevent the covalent binding of EBI to the colchicine binding site on tubulin protein. SMS077 and SMS096 bound to the colchicine binding site with higher affinity than SMS001, SMS182, SMS088 and SMS229. The affinity of the molecules in this assay to the colchicine binding site was lower than colchicine and podophillotoxin. The inventors also showed that SMS077 bound to the colchicine binding site in NALM6 and HELA cells. SMS077 binding affinity was higher than SMS001 in RPMI-8226 and NALM6 cells. These studies confirm a mode of action by which SMS077 and SMS182 induce depolymerization of tubulin by interacting with the colchicine binding site (FIG. 14 and FIGS. 15A-B).

### Example 5

### Induction of Cell Cycle Arrest and Apoptosis

Cell cycle arrest is a shared consequence of MTAs treatment. The effects of cell cycle distribution were determined in RPMI8226 cells treated for 6 and 18 hours with a range of concentrations of the molecules. The cells were stained with propidium iodide and cell cycle distribution was measured using a cytoFLEX flow cytometer.

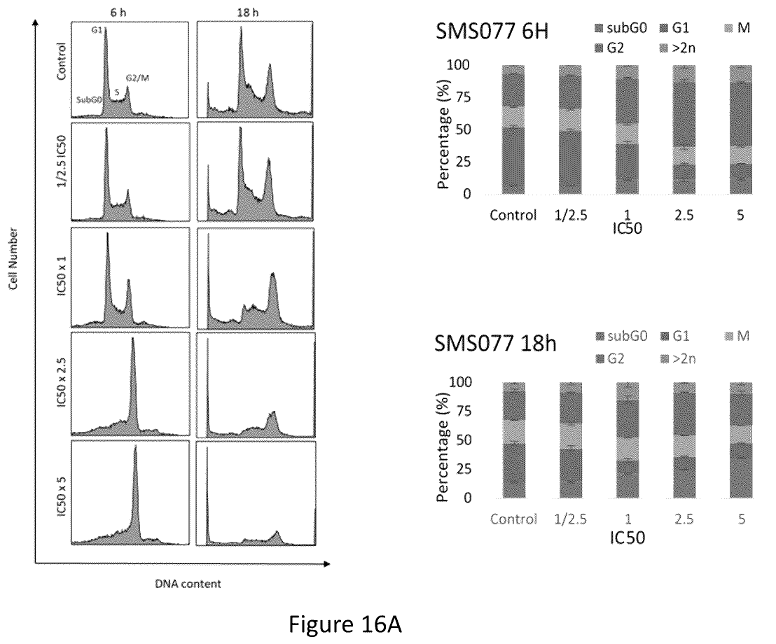

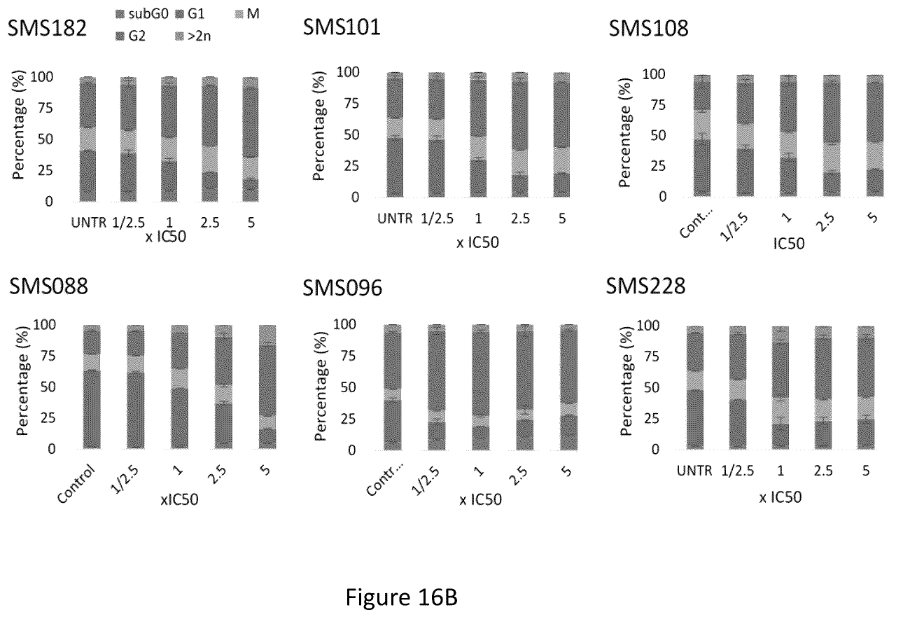

SMS077, SMS182, SMS101, SMS108, SMS088, SMS096, SMS228 led to cell cycle arrest in G2M phase after 6 hours of treatment at IC₅₀, IC₅₀x2.5 and IC₅₀x5 concentrations in a dose dependent manner. The increased in subG0 phase after 18 hours of SMS077 treatment, is indicative of apoptosis activation (FIGS. 16A-B).

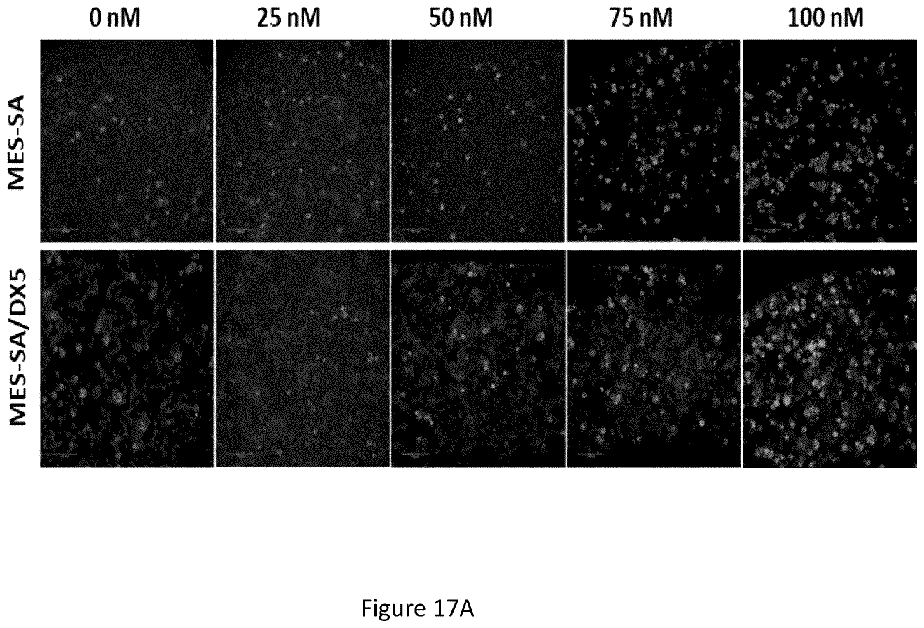

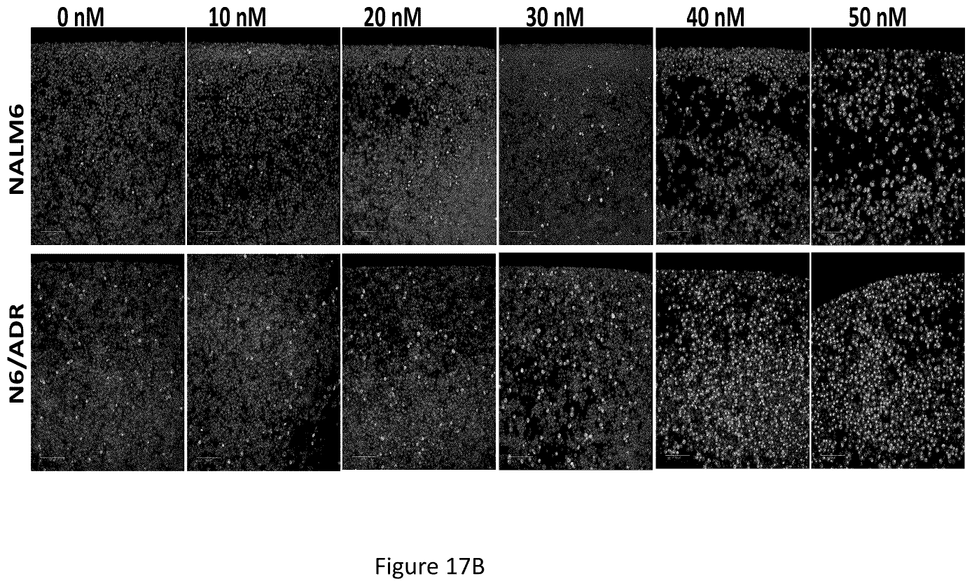

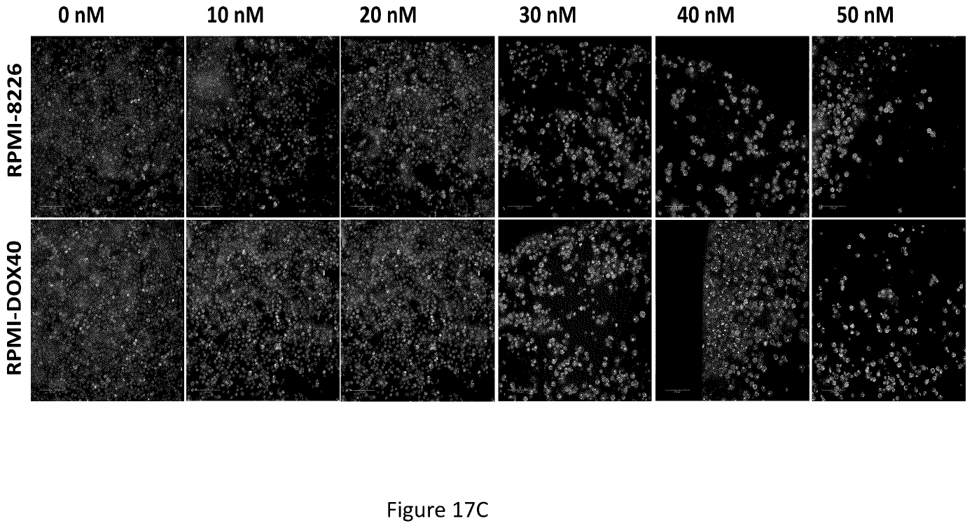

SMS077 induced apoptosis and necrosis of treated MESA/SA and MES-SA/DX5, NALM6 and NALM6/ADR, RPMI8226 and RPMI-DO40 cells was also shown by dose dependent positive staining with the apoptotic marker annexin and with propidium iodide staining indicative of necrosis by immunofluorescence assay (FIGS. 17A-C).

### Example 6

### Inhibition of Cancer Cells Migration In Vitro

Metastasis represent the major cause of cancer-related mortality. Given the active role of microtubules in cell motility and migration, the effect of SMS077 and SMS182 on migration was determined using wound-healing assay. After removing a confluent monolayer of adherent MDA-MB-231 breast cancer cells, BXPC3 prostate cancer cells and HELA cervical adenocarcinoma cells, the remaining cells were treated for 36 h with graded concentrations of SMS077 and SMS182 and compared against untreated control cells. The relative wound density was calculated as ratio of the total occupied scratch area after treatment compared to the total area of removed cells immediately prior to treatment. In the three tested cell lines, the control cells were able to efficiently migrate into the wound channel, recovering 87.6±2.3, 91.2±3.6 and 96.6±1.6% of the area, respectively after 36 h of treatment. Cells treated with SMS077 showed a dose dependent recovery of the wound channel, reaching recovering of the area of 36.8±7.1 for MDA-MB-231 cells, 58.5±3.3 for BXPC3 cells and 25.4±3.9% for HELA cells after 36 hours at a concentration of 100 nM.

Cells treated with SMS182 showed a dose dependent recovery of the wound channel, reaching recovering of the area of 51.5±5.3 MDA-MB-231 cells, 53.6±3.6 BXPC3 cells and 34±3.4% for HELA cells after 36 hours at a concentration of 100 nM.

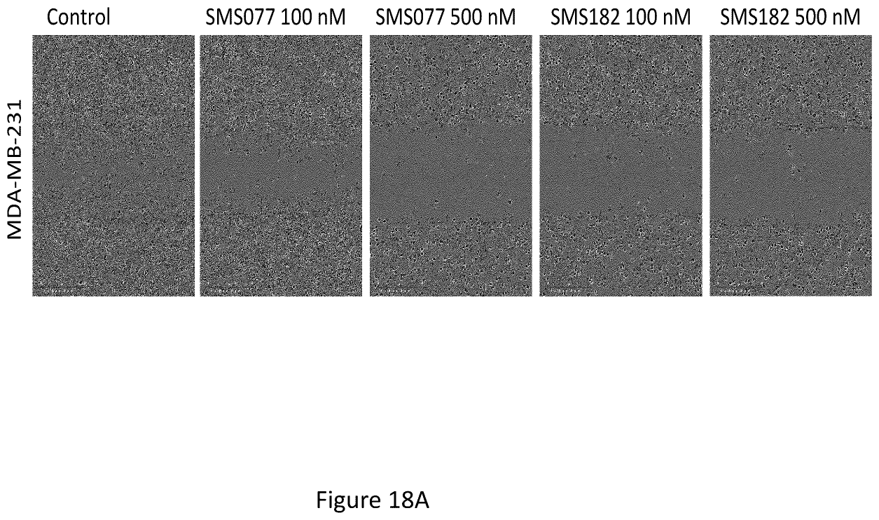

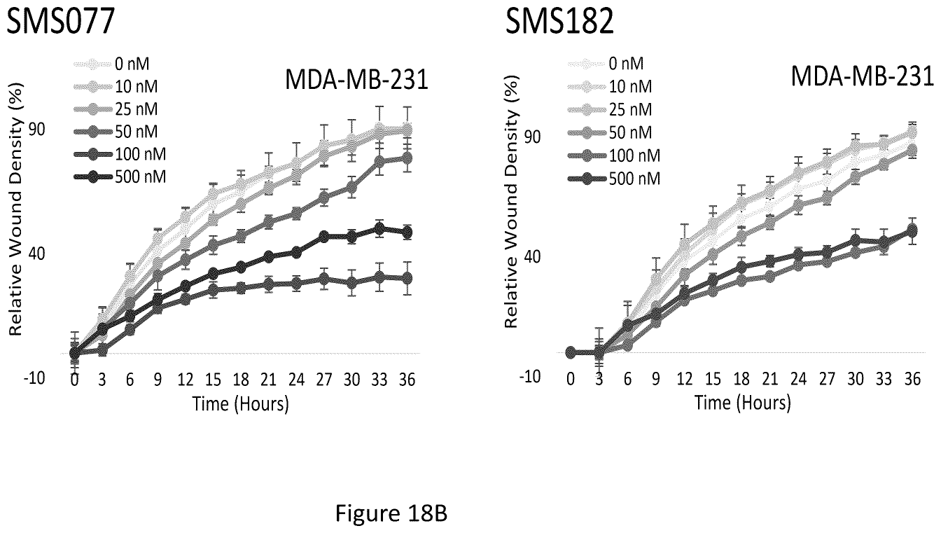

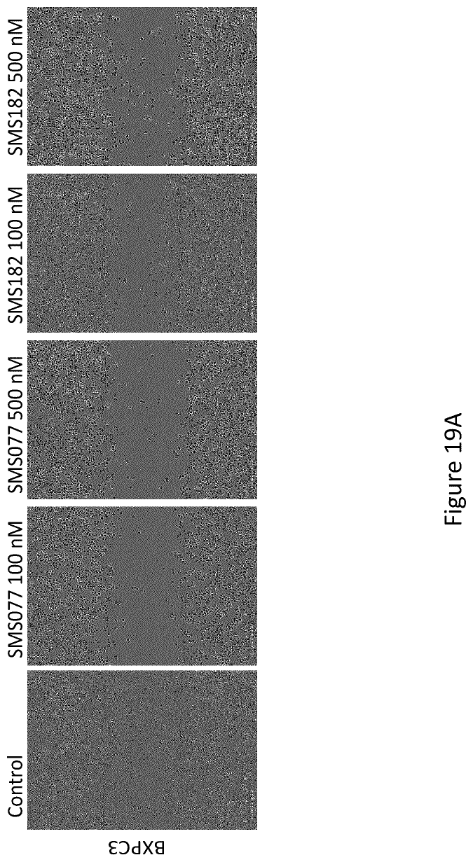

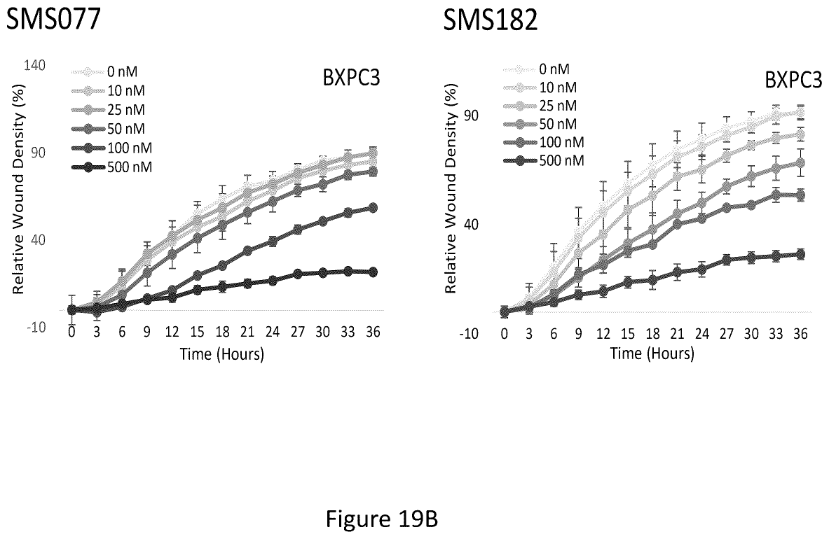

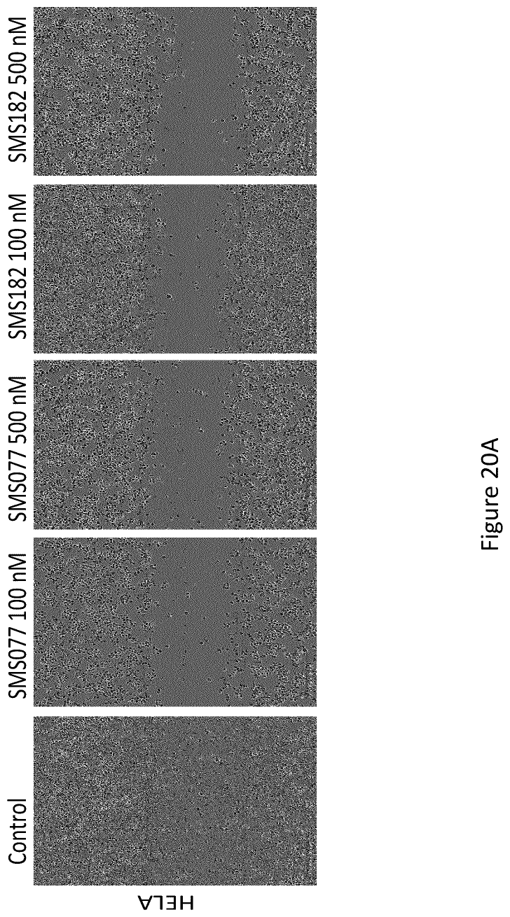

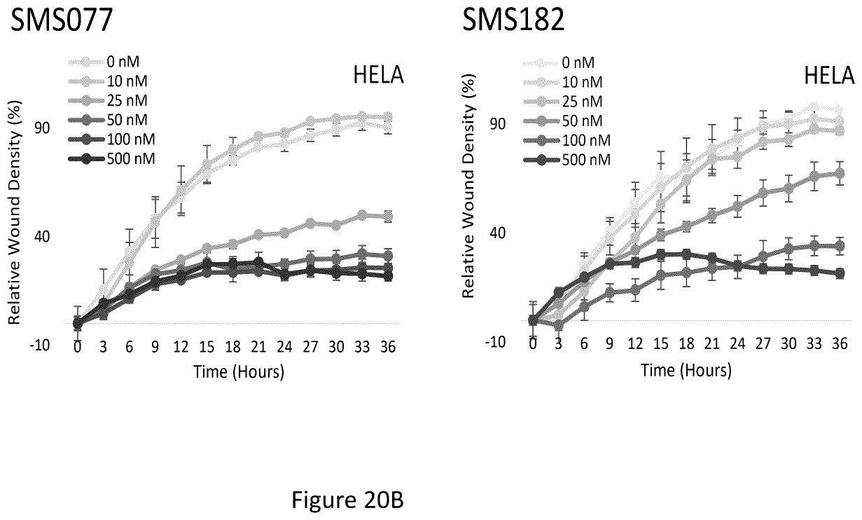

Then, both molecules not only decreased cell viability of cancer cells but also inhibited their migration in a dose dependent manner as compared to control group (FIGS. 18A-B, FIGS. 19A-B and FIGS. 20A-B).

### Example 7

### Tube Formation Inhibition and Vascular-Disrupting Activity In Vitro

The dependence of tumors on neovascularization to grow and subsist, make angiogenesis a potential target to treat cancer. Bevacizumab, a humanized variant of a VEGF neutralizing monoclonal antibody, was the most broadly used anti-angiogenic agent to suppress neovasculature for cancer therapy. By contrast, vascular disrupting agents disrupt existing tumor blood vessels and lead to tumor ischemia and necrosis.

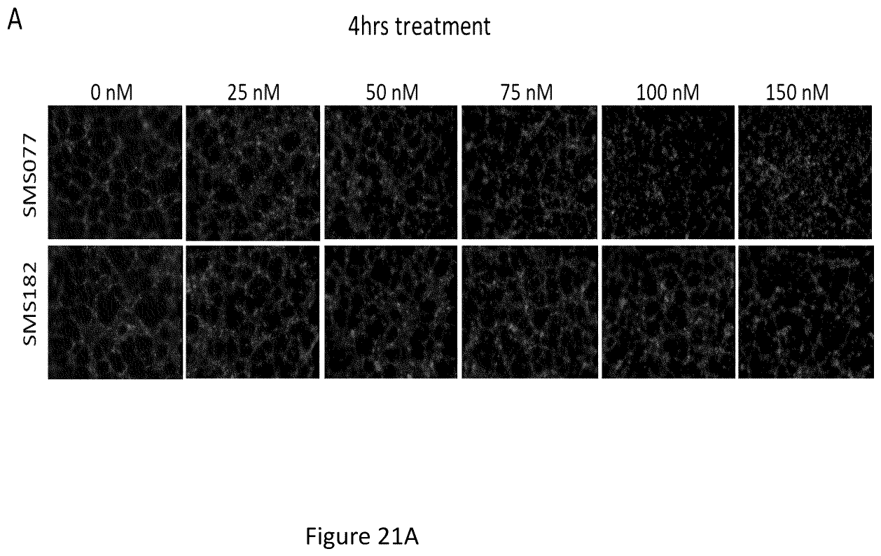

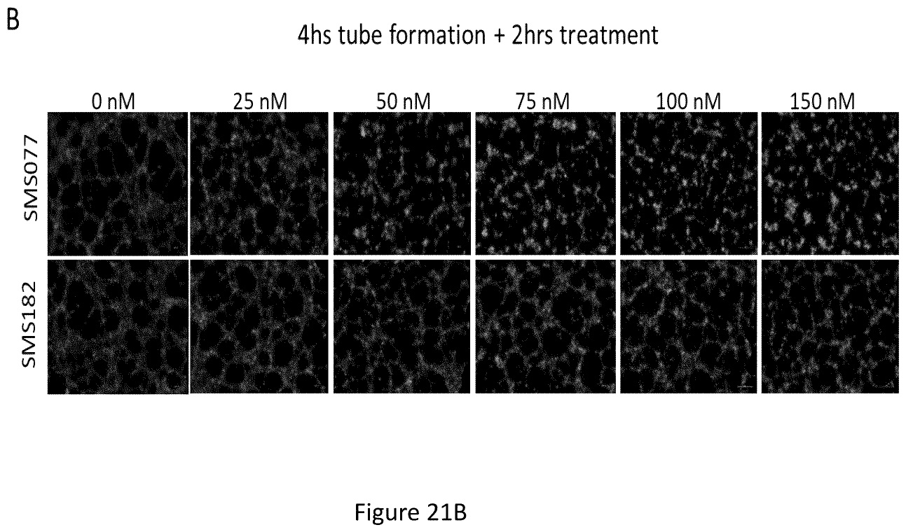

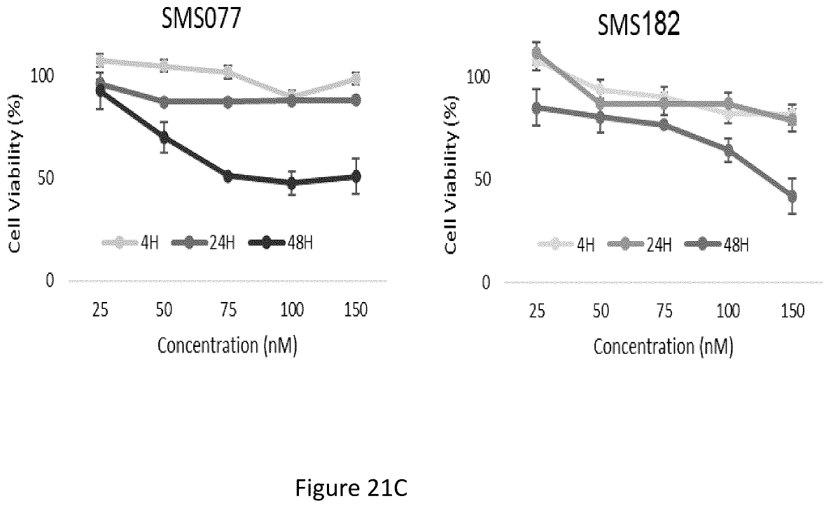

Endothelial cells seeded on Matrigel are able to form three-dimensional capillary structures with a lumen resembling the first angiogenesis steps. The inventors evaluated the effect of SMS077 and SMS182 on inhibition of angiogenesis and disruption of existing vascular tubes (FIGS. 21A-C).

The analysis of anti-angiogenic effect was performed by exposing HUVEC cells to the compounds and seeding them on Matrigel matrix for 4 hours. During this time, control group formed an extensive network lumen-like structure as compared to partially disorganized cells treated with lower doses of SMS077 and SMS182 and disorganized and rounded cells treated with higher doses of the molecules. SMS077 and SMS182 showed a dose dependent inhibition of tubule formation that was significantly inhibited at concentration of 75 nM and 150 nM respectively after 4 hours of treatment.

The analysis of the anti-vascular effect was performed by seeding HUVEC cells on Matrigel matrix, and incubated them until formation of capillary-like tubes, reminiscent of newly formed vessels after 4 hours. Then, the newly formed tube-like structures, were exposed to SMS077 and SMS182 during additional 2 hours. During this time, the in vitro capillary-like tube formation in HUVECs of vehicle control group remain intact, whereas both SMS077 and SMS182 disrupted the integrity of the network in a dose dependent manner.

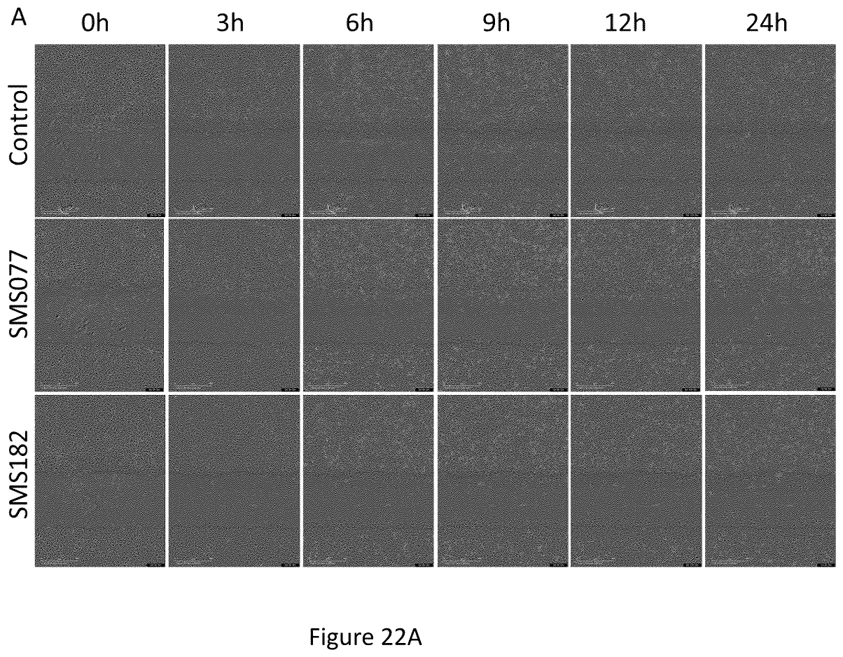

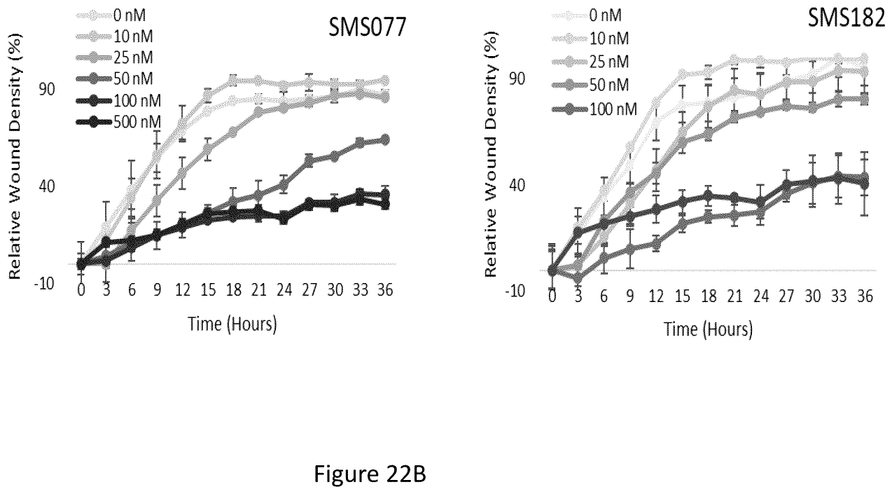

During angiogenesis, endothelial cells proliferate and migrate chemotactically towards angiogenic signals secreted by tumor cells. To determine whether SMS077 and SMS182 could inhibit HUVEC migration, a wound scratch-healing assay was used. Both molecules decreased wound healing density in a dose and time dependent manner. The inhibition of cells migration was reached at lower doses after SMS077 as compared to SMS182 treatment (FIGS. 22A-B). Taken together, these data indicated that SMS077 and SMS182 displayed migration inhibitory and vascular-disrupting activity.

### Example 8

In Vivo Activity from Animal Cancer Model Studies

In vivo anti-cancer efficacy of the molecules was evaluated in a xenograft murine myeloma model. Male SCID mice (6-8 weeks old) were maintained in accordance with Institutional Animal Care Use Committee guidelines. Mice were housed in the Animal Research Facility of Ariel University and experiments were performed in accordance with approved protocols. During the experiment, RPMI8226 myeloma cells in exponential growth were harvested from culture, washed three times with sterile PBS, counted and resuspended in sterile PBS (1.5×107cells mouse/0.1 ml PBS) and subcutaneously injected on the right flank of male C.B-17/IcrHsd-Prkdc-scid, 4-5 weeks old mice. After two weeks of cells injection, when tumors reached a size between 30-60 mm3, mice were assigned into different groups (n=3-5) and started receiving treatment. Body weights were determined twice weekly along with tumor volume {V=[length×(width)2]/2}measurements using electronical caliper. Mice were sacrificed when mean tumor volume of vehicle treated group reached 1500 mm3, or mice appeared moribund to prevent unnecessary morbidity.

### Efficacy Evaluation Calculations

Mean tumor volume represents the average of tumor volume in each treatment group during the experiment.

The weight of the tumors was measured at the end of the experiment and expressed as mean tumor weight per group. Body weight is expressed as mean body mice weight per group.

The individual relative tumor volume (RTV) was defined as Vx/V1, where Vx is the mean tumor volume in mm3 at a given time and V1 at the start of treatment.

Relative tumor volume is determined by calculating RTV (%) (mean RTV of treated group)/(mean RTV of control group)).

Relative tumor volume inhibition is determined by calculating RTVI (%) (1-RTV %)

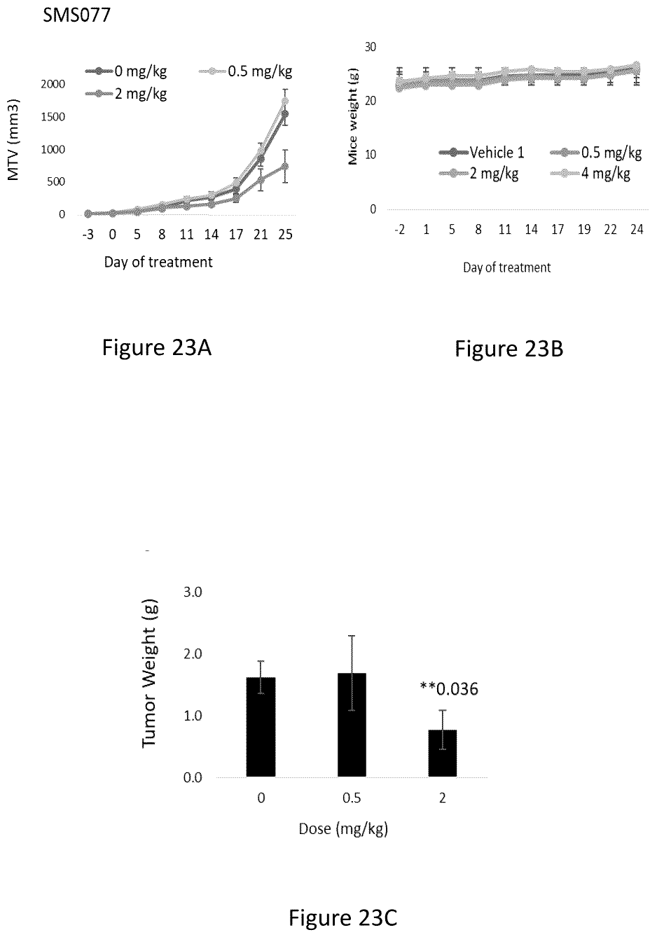

SMS077 reduced the rate of tumor growth in a dose dependent, with smaller effect at 0.5 mg/kg than at 2 mg/kg. Relative tumor volume inhibition of 30% at 0.5 mg/kg and 58% at 2 mg/kg occurred after 21 days of SMS077 (2 mg/kg) treatment. SMS077 decreased tumor volume from 0% at day 8 to 40% at day 11, and 52% at day 25 of treatment as compared to vehicle at the same day. Statistically significant change in tumor weight was detected at the end of the experiment (P=0.036 t test) with a growth inhibition rate of 54%. No significant changes in body weight or other signs of potential toxicity were observed during the experiment (FIGS. 23A-C).

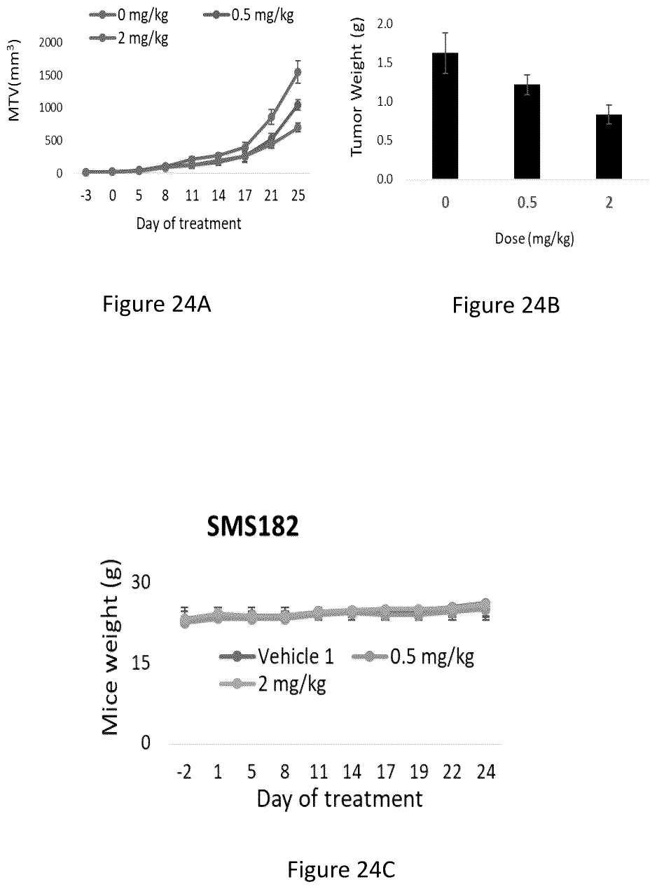

The efficacy of SMS182 was also dose dependent. No significant changes were observed between 2 and 4 mg/kg SMS182 treatment. Relative tumor volume inhibition of 31 and 48% occurred after 21 days of SMS182 treatment at 0.5 and 2-4 mg/kg respectively. Tumor volume decreased from 14% at day 8 to 40% at day 11 and 55% at day 25 of treatment as compared with vehicle at the same day. Statistically significant change in tumor weight was detected at the end of the experiment (P=0-04 t test) reaching a growth inhibition rate of 48%. No significant changes in body weight or other signs of potential toxicity were observed during the experiment (FIGS. 24A-C).

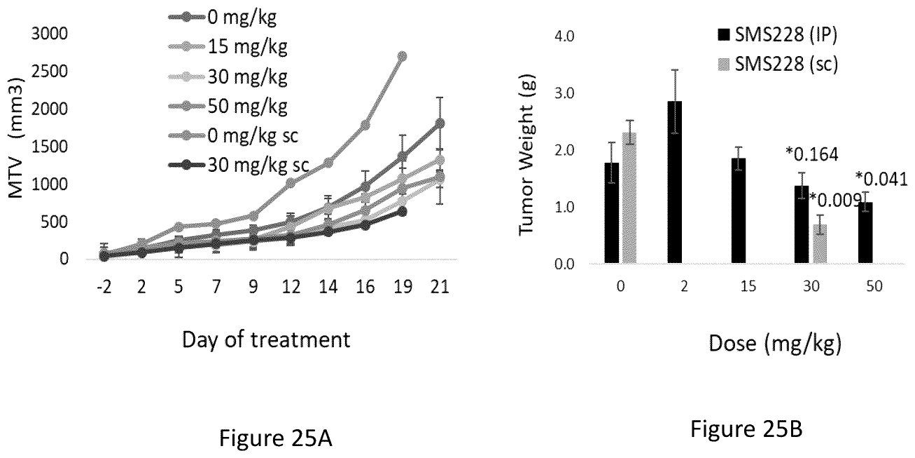

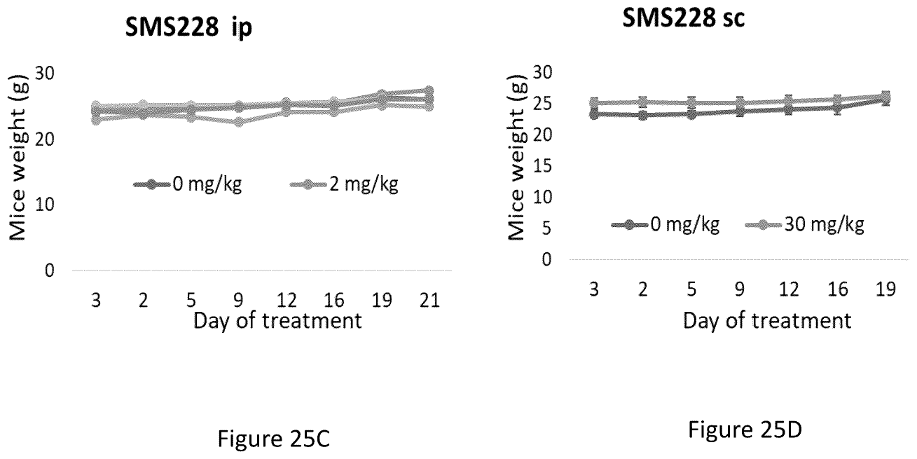

SMS228 given intraperitoneally decreased tumor volume at in a dose dependent manner. Relative tumor volume inhibition of 0, 20, 23 and 42% occurred after 21 days of SMS228 treatment at 15, 30 and 50 mg/kg respectively. Tumor volume decreased 30% from day 7 to 19 and reached a reduction of 40% in day 21 after SMS228 treatment at 50 mg/kg as compared to vehicle treated group at the same day. The SMS228 growth inhibition rate was 44%. No significant changes in body weight or other signs of potential toxicity were observed during the experiment (FIGS. 25A-D).

SMS228 administered subcutaneously reached a relative tumor volume inhibition of 45% after 19 days of treatment at 30 mg/kg. Tumor volume was 56% lower in the treated group at the beginning of the experiment and decreased to 76% as compared to vehicle at day 21 of SMS228 treatment. Statistically significant change in tumor weight was detected at the end of the experiment (P=0.009 t-test) reaching to growth inhibition rate of 70%. No significant changes in body weight or other signs of potential toxicity were observed during the experiment. According to these results 30 mg/kg SMS228 administered subcutaneously had similar efficacy to SMS228 administered at higher doses (50 mg/kg) (FIG. 25D).

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A compound or a salt thereof, wherein said compound is represented by Formula I: 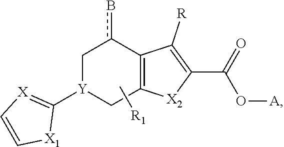
wherein:
R represents a substituent comprising any one of halogen, —C(═O)R′, —C(═O), —NO₂, —CN, —OR′, oxo, imino, —CONH₂, —CONR′₂, —CNNR′₂, —CSNR′₂, —CONH—OH, —CONH—NH₂, NHCOR′, —NHCSR′, —NHCNR′, —NC(═O)OR, —NC(═O)NR′, —NC(═S)OR′, —NC(═S)NR′, —SO₂R′, —SOR′, —SR′, —SO₂OR′, —SO₂N(R′)₂, —NHNR′₂, —NNR′, C₁-C₆ haloalkyl, alkyl, optionally substituted C₁-C₆ alkyl, —NH₂, —N(R′)₂, —NR′₂—NH(C₁-C₆ alkyl), —N(C₁-C₆ alkyl)₂, C₁-C₆ alkoxy, C₁-C₆ haloalkoxy, hydroxy(C₁-C₆ alkyl), hydroxy(C₁-C₆ alkoxy), alkoxy(C₁-C₆ alkyl), alkoxy(C₁-C₆ alkoxy), C₁-C₆ alkyl-NR′2, C₁-C₆ alkyl-SR′, —CONH(C₁-C₆ alkyl), —CON(C₁-C₆ alkyl)₂, —CO₂H, —CO₂R′, —OCOR, —OCOR′, —OC(═O)OR′, —OC(═O)NR′, —OC(═S)OR′, —OC(═S)NR′, a heteroatom, alkyne, an optionally substituted aryl, an optionally substituted bicyclic aromatic ring, an optionally substituted aliphatic ring, an optionally substituted unsaturated aliphatic ring, an optionally substituted polycyclyl, an optionally substituted bicyclic aliphatic ring, optionally substituted C₃-C₁₀ heterocyclyl, an optionally substituted linear C₁-C₁₀ alkyl, optionally substituted linear C₁-C₁₀ heteroalkyl, an optionally substituted branched C₁-C₁₀ alkyl, an optionally substituted C₁-C₁₀ alkyl-aryl, optionally substituted C₁-C₁₀ alkyl-cycloalkyl, optionally substituted C₃-C₁₀ cycloalkyl, an optionally substituted branched C₁-C₇ haloalkyl, an optionally substituted linear C₁-C₇ haloalkyl, an optionally substituted C₁-C₇ alkylhydroxy, an optionally substituted C₁-C₇ alkoxy, or a combination thereof; wherein each R′ independently represents hydrogen, or is selected from the group comprising optionally substituted C₁-C₁₀ alkyl, optionally substituted C₁-C₃₀ alkyl, optionally substituted C₁-C₃₀ alkenyl, optionally substituted C₁-C₃₀ alkynyl, optionally substituted C₃-C₁₀ cycloalkyl, optionally substituted C₃-C₁₀ heterocyclyl, optionally substituted heteroaryl, optionally substituted aryl, hydroxy, amino, —NH₂, —NR′₂—NH(C₁-C₆ alkyl), —N(C₁-C₆ alkyl)₂, C₁-C₆ alkoxy, C₁-C₆ haloalkoxy, hydroxy(C₁-C₆ alkyl), hydroxy(C₁-C₆ alkoxy), alkoxy(C₁-C₆ alkyl), alkoxy(C₁-C₆ alkoxy), C₁-C₆ alkyl-NR′2, C₁-C₆ alkyl-SR′, or a combination thereof,
R₁ represents hydrogen, or one or more substituent(s) each independently comprising any one of: C(═O)R′, —C(═O), halogen, —NO₂, —CN, —OR′, oxo, imino, —CONH₂, —CONR′₂, —CNNR′₂, —CSNR′₂, —CONH—OH, —CONH—NH₂, NHCOR′, —NHCSR′, —NHCNR′, —NC(═O)OR, —NC(═O)NR′, —NC(═S)OR′, —NC(═S)NR′, —SO₂R′, —SOR′, —SR′, —SO₂OR′, —SO₂N(R′)₂, —NHNR′₂, —NNR′, C₁-C₆ haloalkyl, optionally substituted C₁-C₆ alkyl, —NH₂, —N(R′)₂, —NR′₂—NH(C₁-C₆ alkyl), —N(C₁-C₆ alkyl)₂, alkyl, C₁-C₆ alkoxy, C₁-C₆ haloalkoxy, hydroxy(C₁-C₆ alkyl), hydroxy(C₁-C₆ alkoxy), alkoxy(C₁-C₆ alkyl), alkoxy(C₁-C₆ alkoxy), C₁-C₆ alkyl-NR′2, C₁-C₆ alkyl-SR′, —CONH(C₁-C₆ alkyl), —CON(C₁-C₆ alkyl)₂, —CO₂H, —CO₂R′, —OCOR, —OCOR′, —OC(═O)OR′, —OC(═O)NR′, —OC(═S)OR′, —OC(═S)NR′, a heteroatom, alkyne, an optionally substituted heteroaryl, an optionally substituted aryl, an optionally substituted bicyclic aromatic ring, an optionally substituted aliphatic ring, an optionally substituted unsaturated aliphatic ring, an optionally substituted bicyclic aliphatic ring, optionally substituted C₃-C₁₀ heterocyclyl, an optionally substituted polycyclyl, an optionally substituted linear C₁-C₁₀ alkyl, optionally substituted linear C₁-C₁₀ heteroalkyl, an optionally substituted branched C₁-C₁₀ alkyl, an optionally substituted C₁-C₁₀ alkyl-aryl, optionally substituted C₁-C₁₀ alkyl-cycloalkyl, optionally substituted C₃-C₁₀ cycloalkyl, an optionally substituted branched C₁-C₇ haloalkyl, an optionally substituted linear C₁-C₇ haloalkyl, an optionally substituted C₁-C₇ alkylhydroxy, an optionally substituted C₁-C₇ alkoxy, a hydroxy, a heteroatom, a halogen, an alkyne, an alkoxy, an amino, or a combination thereof,
A represents a substituent comprising any one of an optionally substituted linear C₁-C₁₀ alkyl, optionally substituted linear C₁-C₁₀ heteroalkyl, an optionally substituted branched C₁-C₁₀ alkyl, an optionally substituted C₁-C₁₀ alkyl-aryl, an optionally substituted C₁-C₁₀ alkyl-cycloalkyl, an optionally substituted C₃-C₁₀ cycloalkyl, an optionally substituted branched C₁-C₇ haloalkyl, an optionally substituted linear C₁-C₇ haloalkyl, an optionally substituted C₁-C₇ alkylhydroxy, an optionally substituted unsaturated aliphatic ring, an optionally substituted C₃-C₁₀ heterocyclyl, or a combination thereof, [symbol]
represents a single bond or a double bond;
B is selected from O and OH;
X, X₁ and X₂ are independently selected from the group consisting of: S, O, N, and NH;
and Y is selected from N and CH.

2. The compound of claim 1, wherein R represents a substituent comprising any one of —C(═O)R′, —C(═O), halogen, —NO₂, —CN, —OR′, oxo, imino, —CONH₂, —CONR′₂, —CNNR′₂, —CSNR′₂, —CONH—OH, —CONH—NH₂, NHCOR′, —NHCSR′, —NHCNR′, —NC(═O)OR, —NC(═O)NR′, —NC(═S)OR′, —NC(═S)NR′, —SO₂R′, —SOR′, —SR′, —SO₂OR′, —SO₂N(R′)₂, —NHNR′₂, —NNR′, C₁-C₆ haloalkyl, optionally substituted C₁-C₆ alkyl, —NH₂, —N(R′)₂, —NR′₂—NH(C₁-C₆ alkyl), —N(C₁-C₆ alkyl)₂, C₁-C₆ alkoxy, C₁-C₆ haloalkoxy, hydroxy(C₁-C₆ alkyl), hydroxy(C₁-C₆ alkoxy), alkoxy(C₁-C₆ alkyl), alkoxy(C₁-C₆ alkoxy), C₁-C₆ alkyl-NR′2, C₁-C₆ alkyl-SR′, —CONH(C₁-C₆ alkyl), —CON(C₁-C₆ alkyl)₂, —CO₂H, —CO₂R′, —OCOR, —OCOR′, —OC(═O)OR′, —OC(═O)NR′, —OC(═S)OR′, —OC(═S)NR′, a heteroatom, or a combination thereof; wherein R₁ represents hydrogen, or one or more substituent(s) each independently comprising any one of: C(═O)R′, —C(═O), halogen, —NO₂, —CN, —OR′, oxo, imino, —CONH₂, —CONR′₂, —CNNR′₂, —CSNR′₂, —CONH—OH, —CONH—NH₂, NHCOR′, —NHCSR′, —NHCNR′, —NC(═O)OR, —NC(═O)NR′, —NC(═S)OR′, —NC(═S)NR′, —SO₂R′, —SOR′, —SR′, —SO₂OR′, —SO₂N(R′)₂, —NHNR′₂, —NNR, C₁-C₆ haloalkyl, optionally substituted C₁-C₆ alkyl, —NH₂, —N(R′)₂, —NR′₂—NH(C₁-C₆ alkyl), —N(C₁-C₆ alkyl)₂, C₁-C₆ alkoxy, C₁-C₆ haloalkoxy, hydroxy(C₁-C₆ alkyl), hydroxy(C₁-C₆ alkoxy), alkoxy(C₁-C₆ alkyl), alkoxy(C₁-C₆ alkoxy), C₁-C₆ alkyl-NR′2, C₁-C₆ alkyl —SR′, —CONH(C₁-C₆ alkyl), —CON(C₁-C₆ alkyl)₂, —CO₂H, —CO₂R′, —OCOR, —OCOR′, —OC(═O)OR′, —OC(═O)NR′, —OC(═S)OR′, —OC(═S)NR′, a heteroatom, or alkyne; and
wherein said heteroatom is or comprises any one or SR′, OR′, and N(R′)₂.

3. The compound of claim 1, wherein said compound is represented by Formula II:

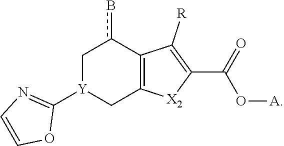

4. The compound of claim 1, wherein X₂ is selected from O and NH; and wherein R is selected from alkyl, C₁-C₆ alkyl, C₁-C₆ haloalkyl, optionally substituted C₁-C₆ alkyl, —NH(C₁-C₆ alkyl), —N(C₁-C₆ alkyl)₂, C₁-C₆ alkoxy, C₁-C₆ haloalkoxy, hydroxy(C₁-C₆ alkyl), hydroxy(C₁-C₆ alkoxy), alkoxy(C₁-C₆ alkyl), alkoxy(C₁-C₆ alkoxy), C₁-C₆ alkyl-NR′₂, and C₁-C₆ alkyl —SR′.

5. The compound of claim 1, wherein said compound is any one of:

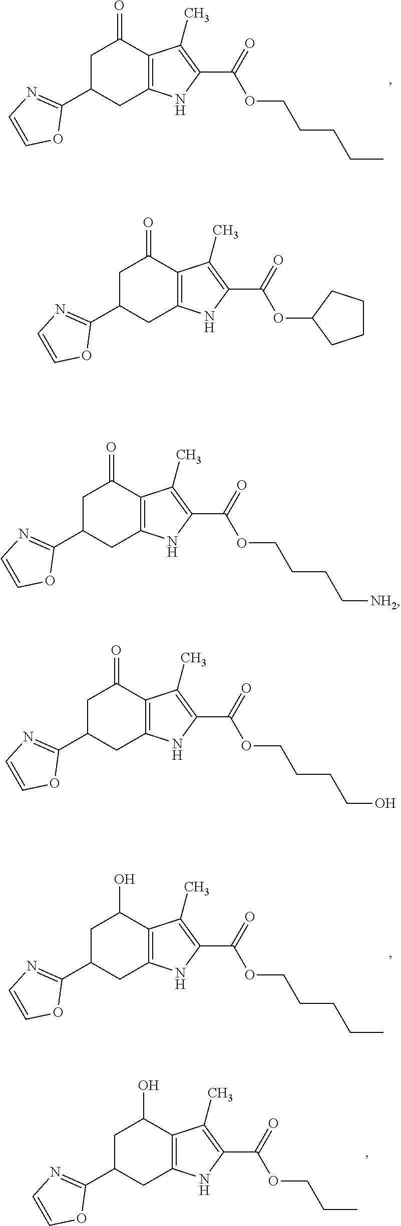 [SMILES: CCCCCOC(=O)c1[nH]c2c(c1C)C(=O)CC(c1ncco1)C2 ; Cc1c(C(=O)OC2CCCC2)[nH]c2c1C(=O)CC(c1ncco1)C2 ; Cc1c(C(=O)OCCCCN)[nH]c2c1C(=O)CC(c1ncco1)C2 ; Cc1c(C(=O)OCCCCO)[nH]c2c1C(=O)CC(c1ncco1)C2 ; CCCCCOC(=O)c1[nH]c2c(c1C)C(O)CC(c1ncco1)C2 ; CCCOC(=O)c1[nH]c2c(c1C)C(O)CC(c1ncco1)C2]

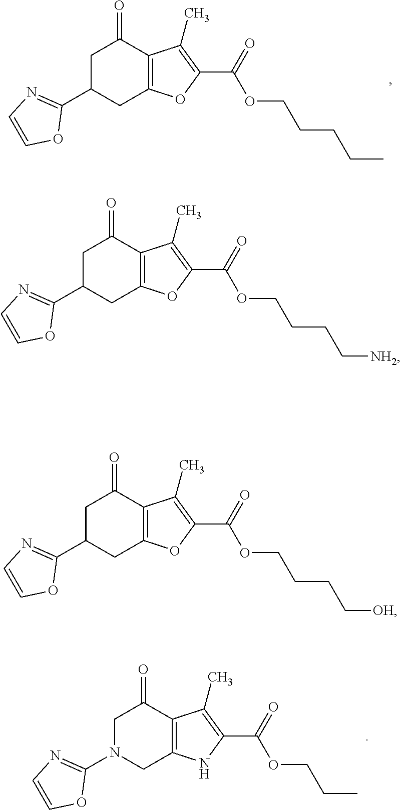 [SMILES: CCCCCOC(=O)c1oc2c(c1C)C(=O)CC(c1ncco1)C2 ; Cc1c(C(=O)OCCCCN)oc2c1C(=O)CC(c1ncco1)C2 ; Cc1c(C(=O)OCCCCO)oc2c1C(=O)CC(c1ncco1)C2 ; CCCOC(=O)c1[nH]c2c(c1C)C(=O)CN(c1ncco1)C2]

6. A pharmaceutical composition, comprising the compound of claim 1 and a pharmaceutically acceptable carrier.

7. (canceled)

8. (canceled)

9. (canceled)

10. A method for preventing or treating an angiogenesis-associated disease or disorder in a subject, comprising administering to said subject the pharmaceutical composition of claim 6, thereby preventing or treating an angiogenesis-associated disease or disorder in a subject.

11. The method of claim 10, wherein said angiogenesis-associated disease or disorder is cancer.

12. A method for treating or preventing development of cancer in a subject in need thereof, comprising administering to said subject the pharmaceutical composition of claim 6.

13. The method of claim 12, wherein said cancer is selected from the group consisting of lung cancer, breast cancer, osteosarcoma, neuroblastoma, colon adenocarcinoma, chronic myelogenous leukemia (CML), acute lymphoblastic leukemia, acute monocytic leukemia, acute myeloid leukemia (AML), acute promyelocyte leukemia (APL), sarcoma, myxoma, rhabdomyoma, fibroma, lipoma, teratoma; bronchogenic carcinoma, alveolar carcinoma, bronchial adenoma, sarcoma, lymphoma, chondromatous hamartoma, mesothelioma, esophageal cancer, stomach cancer, pancreatic cancer, small bowel cancer, large bowel cancer; kidney cancer, bladder cancer, urethra cancer, prostate cancer, testis cancer; hepatoma, cholangiocarcinoma, hepatoblastoma, angiosarcoma, hepatocellular adenoma, hemangioma, osteogenic sarcoma, fibrosarcoma, malignant fibrous histiocytoma, chondrosarcoma, Ewing's sarcoma, malignant lymphoma, multiple myeloma, malignant giant cell tumor chordoma, osteochronfroma, benign chondroma, chondroblastoma, chondromyxofibroma, osteoid osteoma, giant cell tumors, cancer of the skuli, meninges cancer, brain cancer, spinal cord cancer, uterus cancer, cervical cancer, cancer of the ovaries, vulva cancer, vagina cancer, Hodgkin's disease, non-Hodgkin's lymphoma, malignant melanoma, basal cell carcinoma, squamous cell carcinoma, Karposi's sarcoma, moles dysplastic nevi, lipoma, angioma, uterine sarcoma, cervix adenocarcinoma, ovary cancer, colorectal adenocarcinoma and dermatofibroma.

14. The method of claim 10, wherein said administering is by an oral administration, a topical administration, a systemic administration or any combination thereof.
